(19) United States
(12) Patent Application Publication
(10) Pub. No.: US 2004/0152905 A1
(43) Pub. Date: Aug. 5, 2004

(54) Universal building blocks and support media for synthesis of oligonucleotides and their analogs

(76) Inventors: Andrei P. Guzaev, Vista, CA (US); Muthiah Manoharan, Weston, MA (US)

Correspondence Address: Choate, Hall & Stewart, Exchange Place, 53 State Street, Boston, MA, 02109, US

(21) Appl. No.: 10/770,226

(22) Filed: Feb. 2, 2004

Related U.S. Application Data
(60) Provisional application No. 60/444,363, filed on Jan. 31, 2003.

(51) Int. Cl. C07D 487/02

(52) U.S. Cl. 548/453; 549/463

(57) ABSTRACT

Compounds for the synthesis of oligomeric compounds, particularly oligonucleotides and chemically modified oligonucleotide analogs, are provided. In addition, methods for functionalization of a support medium with a first monomeric subunit and methods for the synthesis of oligomeric compounds utilizing the novel compounds bound to support media are provided.

### RELATED APPLICATIONS

This application claims the benefit of U.S. Provisional Application No. 60/444,363, filed on Jan. 31, 2003, the entire teachings of which are incorporated herein by reference.

### FIELD OF THE INVENTION

This invention is directed in one aspect to compounds useful in the preparation of novel universal building blocks and support media. The universal building blocks and support media thus prepared are useful in the preparation of oligomeric compounds.

### BACKGROUND OF THE INVENTION

Support bound oligonucleotide synthesis relies on sequential addition of nucleotides to one end of a growing chain. Typically, a first nucleoside is attached to an appropriate support medium such as a glass bead support and activated phosphorus compounds (typically nucleotide phosphoramidites, also bearing appropriate protecting groups) are added stepwise to elongate the growing oligonucleotide. When the chain elongation is completed, the oligonucletide is cleaved from its support and protecting groups are removed. Additional methods fro support bound synthesis methods may be found in Caruthers U.S. Pat. Nos. 4, 415,732; 4,458,066, 4,500,707; 4,668,777; 4,973,679; and 5,132,418; and Koster U.S. Pat. No. 4,725,677 and Re. 34,069.

In carrying out standard oligonucleotide syntheses, workers minimally need to maintain a supply of eight different nucleoside-loaded supports for DNA and RNA syntheses, each prederivatized with a separate nucleoside corresponding to the 3′ terminus of the desired oligomer (adenosine, guanosine, cytidine, uridine, deoxyadenosine, deoxyguanosine, deoxycytidine, and thymidine). If a modified nucleoside is desired at the 3′-terminus then additional prederivatized supports are required. Typically, the first nucleoside is covalently bound to a support media by an ester linkage, for instance succinate or hydroquinone-O,O′-diacetate linkers. Furthermore, certain unusual nucleosides are available only as phosphoramidite building blocks but not as supports.

A universal support is a support that may be used as a starting point for oligonucleotide synthesis regardless of the nucleoside species at the 3′-end of the sequence. A universal support has broad applications and remedies the aforementioned deficiencies of standard oligonucleotide synthesis procedures because only one support is needed to carry out the oligonucleotide synthesis regardless of what base is desired at the 3′-end. This simplifies the synthetic strategy, reduces the number of required reagents in inventory and the likelihood of errors in parallel synthesis applications.

We previously prepared a non-nucleosidic universal solid support based on a conformationally pre-organized cyclic 1,2-diol covalently attached to the controlled pore glass via a stable linkage [Guzaev, A. P.; Manoharan, M. J. Amer. Chem. Soc. 2003, 125, 2380-2381; U.S. Pat. No. 6,653,468]. One of the hydoxy functions in said solid support is used for the assemblying of oligonucleotide compounds; the other one remains protected until the end of the chain assembly. In the following step, the oligomeric compound having free 3′-hydroxy group at the 3′terminal nucleoside residue is released from the solid support via a dephosphorylation reaction.

In a different approach, Ngo used 2-(4-monomethoxytrityloxy)phenol as a universal linker attached to the controlled pore glass via a cleavable succinyl moiety [Ngo, N. Q. PCT Int. Appl. WO 00/69878]. Following the oligonucleotide synthesis, the support-bound oligomeric compound was treated with ammonium hydroxide, which released to the solution the 3′-dephosphorylated oligonucleotide and ortho-quinone as a side product. The latter compound is a known mutagen capable of reacting with nucleic bases to give undesired modified oligonucleotides which have to be removed by chromatographic purification.

Some researchers have employed derivatized glass supports with 2′(3′)-O-benzoyluridine 5′-O-succinate so that the uridine moiety is linked to the glass via a succinate linkage [deBear et al., Nucleosides and Nucleotides 1987, 6, 821-830]. Oligonucleotide synthesis takes place by adding nucleotide monomers to the 2′ or 3′ position of the uridine. Following the synthesis, the newly synthesized oligonucleotide is released from the glass, deprotected and cleaved from the uridinyl terminus in one reaction. Since it is cleaved from the solid support in the cleaving reaction, the uridinyl functionality is no longer available for subsequent oligonucleotide syntheses.

In a similar approach, Crea et al. prepared the dimer 5′-O-p-chlorophenylphospo-2′-(3′)-O-acetyluridinyl-[2′(3′)→3′]-5′-O-dimethoxytritylthymidine p-cholrophenylester and attached the dimer to cellulose via a phosphate linkage. The 5′ position of the thymidine is available for oligonucleotide attachment and synthesis. [Crea et al., Nucleic Acids Research 1980, 8, 2331]. Aqueous concentrated ammonia is used for the release of the synthesized oligonucleotide from the cellulose leaving the uridine portion of the dimer attached to the cellulose. Although Crea et al. utilized the reactive vicinal groups of the uridine as the release site for the oligonucleotide from the uridine the solid support suggested in this reference is not truly a universal solid support because the 3′-terminal nucleoside is incorporated in the solid support reagent and a different support is required for oligonucleotides incorporating a different first nucleoside.

Schwartz et al. attached an adapter, 2′-(3′)-O-dimethoxytrityl-3′-(2′)-O-benzoyluridine-5′-O-(2-cyanoethyl-N,N-diisopropylphosphoramidite), to a thymidine derivatized polystyrene and synthesized an oligonucleotide from the O-dimethoxytrityl position f the uridine [Schwartz et al., Tetrahedron Letters 1995, 36, 1, 27-30]. While this approach provides a universal solid support for oligonucleotide synthesis, cleavage releases both the adapter and the thymidine from the support and then the synthesized oligonucleotide from the uridine. Thus, uridine linker must be removed as an impurity from the reaction mixture.

In a similar approach, Kumarev attached a 2′,3′-di-O-(methoxyethylydene)inosine to the controlled pore glass via a cleavable linker [Kumarev, V. PCT Int. Appl. WO 01/96357]. Similarly to the other examples of using nucleosidic moieties as universal linkers, the cleavage requires a prolonged heating of oligomeric compound in aggressive media and contaminates the product with undesired nucleosidic impurity.

Some universal supports require cleavage under conditions supplemental to ammonium hydroxide, [Lyttle et al., Nucleic Acids Research 1996, 24, 14, 2793-2798; Azhayev, A. V.; Antopolsky, M. L. Tetrahedron 2001, 57, 4977-4986] making them less useful in many conventional syntheses where ammonium hydroxide is used as cleavage reagent.

The compounds, compositions, and processes of the invention provide novel universal non-nucleosidic building blocks and support media useful for preparing oligomeric compounds, including oligonucleotides and oligonucleotide mimetics, which do not require a pre-derivatization of to the solid support media with a 3′-terminal nucleoside.

### BRIEF SUMMARY OF THE INVENTION

In one embodiment, the invention is directed to compounds of Formula I:

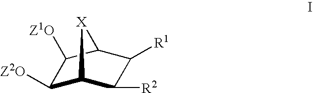

wherein:

X is O or NR³;

R³ is -L-sm, alkyl, —C(═O)alkyl, —C(═O)aryl, —C(═O)NH-alkyl, —C(═O)NH-aryl or an amino protecting group;

L is a linking moiety;

sm is a support medium;

R¹ and R² are independently H, alkyl, —C(═O)—R⁴; or R¹ and R² are fused to form a ring structure so that R¹+R² is —C(═O)—N(R⁵)—C(═O)—; or R¹ and R² together with the carbon atoms they are attached to form a substituted or unsubstitute cycloalkyl, a substituted or unsubstituted aryl, a substituted or unsubstituted heterocycloalkyl, or a substituted or unsubstituted heteroaryl; or one of R₁ and R₂ is -L-sm and the other of R¹ and R² is H, O—C(═O)R⁶, or —C(═O)—R⁴;

R⁴ is —O(alkyl), —O(benzyl), —O(alkoxyalkyl), or —N(J¹)J²;

J¹ is H or alkyl;

J² is H, alkyl, benzyl, alkoxyalkyl, —(CH₂)ₙ—O-L-sm, or a nitrogen-protecting group;

n is an integer from 0 to about 12;

or J¹ and J² together with the nitrogen atom they are attached to form a heteroaryl or heterocycloalkyl;

R⁵ is alkyl, aryl, benzyl, alkoxyalkyl, —(CH₂)ₙ-L-sm, or nitrogen-protecting group;

R⁶ is CH₂-G¹;

Z¹ and Z² are independently H, or orthogonal hydroxy protecting groups; or one of Z¹ or Z² is H and the other of Z¹ or Z² is —C(═O)CH₂G¹; or one of Z¹ or Z² is H or hydroxy protecting group and the other of Z¹ or Z² is -L-sm;

or Z₁ and Z₂ together with the oxygen atoms they are attached to and the carbon atoms the oxygen atoms are attached to form a ring structure wherein Z¹+Z² is —C(OAlkyl)(CH₂G¹)-;

G¹, for each occurrence, is independently, H, alkyl, aryl, acetyl, acetonyl, or an electron-withdrawing group;

provided that when one of R¹ or R² is -L-sm and the other of R¹ and R² is O—C(═O)R⁶ or —C(═O)—R⁴, then Z¹ and Z² together with the oxygen atoms they are attached to and the carbon atoms the oxygen atoms are attached to form a ring structure wherein Z¹+Z² is —C(OAlkyl)(CH₂G¹)-; and provided that the compound includes one -L-sm.

Preferably, L is —C(═O)—, —CH₂OC(═O)—, —O—C(═O)—, —P(OR⁷)(═O)—, or —P(OR⁷)(═S)—. R⁷ is alkyl, cycloalkyl, or —P[O(CH₂)₂CN](═O)—, or —P[O(CH₂)₂CN](═S)—.

The term “alkyl group”, as used in this application, refers to a linear hydrocarbon chain having 1 to about 24 carbon atoms and isomeric forms thereof. Preferred alkyl groups include methyl, ethyl, propyl, 1-methylethyl (isopropyl), butyl, 1-methylpropyl (sec-butyl), 2-methylpropyl (isobutyl), 1,1-dimethylethyl (tert-butyl).

The term “cycloalkyl group”, refers to a cyclic hydrocarbon having from 3 to about 20 carbon atoms.

The term alkylene refers to an alkyl group that has at least two points of attachment to at least two moieties (e.g., methylene, ethylene, isopropylene, etc.).

The term “aryl group”, as used in this application, refers to a monovalent aromatic carbocyclic group of from 6 to about 24 carbon atoms. Preferred aryl groups include phenyl, 1-naphtyl, and 2-naphthyl.

An arylalkyl group refers to an aryl group that is attached to another moiety via an alkylene linker.

The term “heteroaryl,” as used herein, means an aromatic heterocycle having from about 5 to about 24 ring atom in which 1, 2, 3 or 4 ring atoms are heteroatoms selected from nitrogen, sulfur or oxygen. A heteroaryl may be fused to one or two rings, such as a cycloalkyl, a heterocycloalkyl, an aryl, or a heteroaryl. The point of attachment of a heteroaryl to a molecule may be on the heteroaryl, cycloalkyl, heterocycloalkyl or aryl ring, and the heteroaryl group may be attached through carbon or a heteroatom. Examples of heteroaryl groups include imidazolyl, furyl, pyrrolyl, thienyl, oxazolyl, thiazolyl, isoxazolyl, thiadiazolyl, oxadiazolyl, pyridinyl, pyrimidyl, pyrazinyl, pyridazinyl, quinolyl, isoquniolyl, indazolyl, benzoxazolyl, benzofuryl, benzothiazolyl, indolizinyl, imidazopyridinyl, pyrazolyl, triazolyl, isothiazolyl, oxazolyl, tetrazolyl, benzimidazolyl, benzoxazolyl, benzothiazolyl, benzothiadiazolyl, benzoxadiazolyl, indolyl, tetrahydroindolyl, azaindolyl, imidazopyridyl, qunizaolinyl, purinyl, pyrrolo[2,3]pyrimidyl, pyrazolo[3,4]pyrimidyl or benzo(b)thienyl each of which is optionally substituted. Heteroaryl groups may be substituted or unsubstituted.

A heterocycloalkyl refers to a non-aromatic ring which contains one or more oxygen, nitrogen or sulfur (e.g., morpholine, piperidine, piperazine, pyrrolidine, and thiomorpholine). A heterocycloalkyl can have 3 to about 24 ring atoms and may be substituted or unsubstituted.

Suitable substituents for an alkyl, a cycloalkyl, a heterocycloalkyl, an aryl, and a heteroaryl include any substituent that is stable under the reaction conditions used in the method of the invention. Examples of substituents for an aryl or a heteroaryl include an aryl (e.g., phenyl), an arylalkyl (e.g., benzyl), nitro, cyano, halo (e.g., fluorine, chlorine and bromine), alkyl (e.g., methyl, ethyl, isopropyl, cyclohexyl, etc.) haloalkyl (e.g., trifluoromethyl), alkoxy (e.g., methoxy, ethoxy, etc.), hydroxy, —NR₁₀R₁₁, —NR₁₀C(O)R₁₂, —C(O)NR₁₀R₁₁, —C(O)R₁₀, —C(O)OR₁₀, —OC(O)R₁₂, wherein R₁₀ and R₁₁ for each occurrence are, independently, —H, an alkyl, an aryl, or an arylalkyl; and R₁₂ for each occurrence is, independently, an alkyl, an aryl, or an arylalkyl.

Alkyl, cycloalkyl, or heterocycloalkyl groups may include any of the above listed substituents and may also be substituted with ═O and ═S.

A linking moiety can be any group of atoms that are linked together and have two points of attachment. A linking moiety typically is composed of carbon, hydrogen, nitrogen, phosphorus, oxygen and sulfer atoms and preferably has from one to about 24 consecutively linked atoms. In a preferred embodiment, a linking group is —C(═O)—, —CH₂OC(═O)—, —O—C(═O)—, —P(OR⁷)(═O)—, or —P(OR⁷)(═S)—.

In one preferred embodiment, the invention is directed to compounds of Formula II:

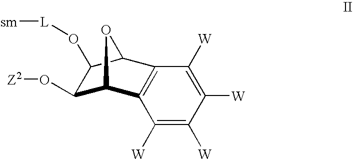

wherein:

L and sm are defined as above;

Z² is 4,4′-dimethoxytrityl group, 4,4′,4″-trimethoxytrityl group, or H; and

W, for each occurrence, is independently H or a halogen atom.

Preferably, W is a halogen atom. More preferably yet, W is F. More preferably, L is —C(═O)—.

In another preferred embodiment, the invention is directed to compounds of Formula III:

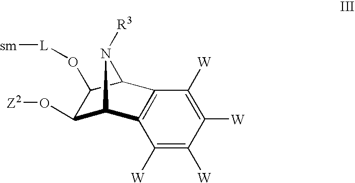

wherein:

L and sm are defined as above;

R³ is alkyl, —C(═O)alkyl, —C(═O)NH(Alkyl), —C(═O)NH(Aryl) or a nitrogen protecting group;

Z² is 4,4′-dimethoxytrityl group, 4,4′,4″-trimethoxytrityl group, or H; and

W, for each occurrence, is independently H or a halogen atom.

Preferably, W is a halogen atom. More preferably yet, W is F. More preferably, L is —C(═O)—.

In yet another preferred embodiment, the invention is directed to compounds having one of Formulas IVa and IVb:

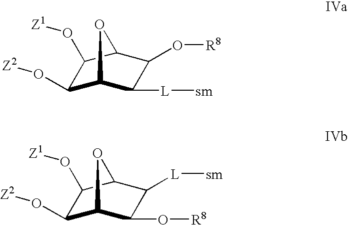

wherein:

L and sm are defined as above;

R⁸ is —C(═O)CH₂-G¹

one of Z¹ and Z² is —C(═O)CH₂-G¹ and the other of Z¹ and Z² is 4,4′-dimethoxytrityl group, 4,4′,4″-trimethoxytrityl group, or H; or Z¹ and Z² together with the oxygen atoms they are attached to and the carbon atoms the oxygen atoms are attached to form a ring structure so that Z¹+Z² is —C(OAlkyl)(CH₂G¹)—.

Preferably, L is —C(═O)—. Preferably, G¹ is H, Cl, acetyl, acetonyl, OCH₃, or —OC₆H₅.

In a more preferred embodiment, the invention is directed to compounds having one of Formulas Va and Vb:

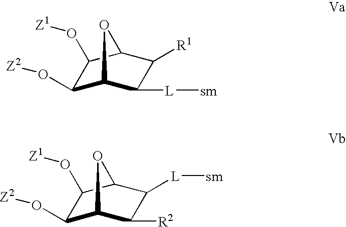

wherein:

L and sm are defined as above;

R¹ and R² are each, independently, H or —C(═O)—R⁴;

one of Z¹ or Z² is H and the other of Z¹ or Z² is —C(═O)CH₂G¹; or Z¹ and Z² together with the oxygen atoms they are attached to and the carbon atoms the oxygen atoms are attached to form a ring structure so that Z¹+Z² is —C(OAlkyl)(CH₂G¹)-.

Preferably, L is —C(═O)—, —CH₂OC(═O)—, —O—C(═O)—, —P(OR⁷)(═O)—, or —P(OR⁷)(═S)—; R⁷ is alkyl, cycloalkyl, or —P[O(CH₂)₂CN](═O)—, or —P[O(CH₂)₂CN](═S)—.

Preferably, G¹ is H, acetyl, acetonyl, Cl, OCH₃, or —OC₆H₅.

In a more preferred embodiment, the invention is directed to compounds having one of Formulas VIa and VIb:

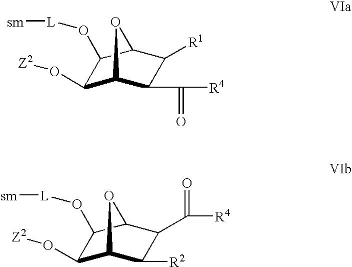

wherein:

L and sm are defined as above;

R¹ and R² are each, independently, H or —C(═O)—R⁴; and

Z² is 4,4′-dimethoxytrityl group, 4,4′,4″-trimethoxytrityl group, or H.

Preferably, L is —C(═O)—.

In an even more preferred embodiment, the invention is directed to compounds of Formula VII:

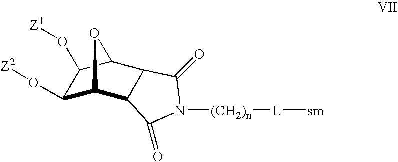

wherein:

sm is defined as above;

L is —OC(═O)—, —C(═O)— or —OP(OR⁷)(═Y)—;

R⁷ is a negative charge, alkyl, cycloalkyl, or phosphate protecting group;

Y is O or S; and

one of Z¹ and Z² is —C(═O)CH₂-G¹ where G¹ is H, an alkyl group, or an electron-withdrawing group and the other of Z¹ and Z² is 4,4′-dimethoxytrityl group, 4,4′,4″-trimethoxytrityl group, or H; or Z¹ and Z² together with the oxygen atoms they are attached to and the carbon atoms the oxygen atoms are attached to form a ring structure so that Z¹+Z² is —C(OAlkyl)(CH₂G¹)-.

Preferably, G¹ is H, Cl, acetyl, acetonyl, OCH₃, or —OC₆H₅.

In another even more preferred embodiment, the invention is directed to compounds of Formula VIII:

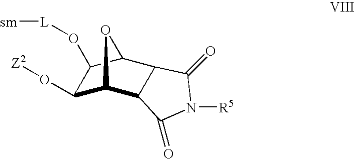

wherein:

L, sm and R⁵ are defined as above;

Z² is 4,4′-dimethoxytrityl group, 4,4′,4″-trimethoxytrityl group, or H.

Preferably, R⁵ is methyl, ethyl, propyl, iso-propyl, phenyl, or benzyl group. Preferably, L is —C(═O)—.

In another embodiment, the invention is directed to compounds of formula Ia:

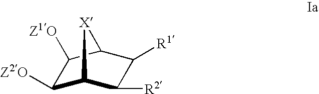

wherein:

X′ is O or NR^{3′};

R^{3′} is -L-R⁹, alkyl, —C(═O)alkyl, —C(═O)aryl, —C(═O)NH-alkyl, —C(═O)NH-aryl or an amino protecting group;

L is a linking moiety;

R⁹ is —X²—P(X₃R⁷)NJ³J⁴;

X² and X³ are each, independently, O or S;

R⁷ is a negative charge, alkyl, cycloalkyl or phosphate protecting group;

J³ and J⁴ are each, independently, and alkyl, a cycloalkyl, or an arylalkyl, or J³ and J⁴ together with the nitrogen atom they are attached to form a heteroaryl or heterocycloalkyl;

R^{1′} and R^{2′} are independently H, alkyl, —C(═O)—R⁴; or R^{1′} and R^{2′} are fused to form a ring structure so that R^{1′}+R^{2′} is —C(═O)—N(R⁵)—C(═O)—; or R^{1′} and R^{2′} together with the carbon atoms they are attached to form a substituted or unsubstitute cycloalkyl, a substituted or unsubstituted aryl, a substituted or unsubstituted heterocycloalkyl, or a substituted or unsubstituted heteroaryl; or one of R^{1′} and R^{2′} is -L-R⁹ and the other of R¹ and R² is H, O—C(═O)R⁶, or —C(═O)—R⁴;

R⁴ is —O(alkyl), —O(benzyl), —O(alkoxyalkyl), or —N(J¹)J²;

J¹ is H or alkyl;

J² is H, alkyl, benzyl, alkoxyalkyl, —(CH₂)ₙ—O-L-sm, or a nitrogen-protecting group;

n is an integer from 0 to about 12;

or J¹ and J² together with the nitrogen atom they are attached to form a heteroaryl or heterocycloalkyl;

R⁵ is alkyl, aryl, benzyl, alkoxyalkyl, —(CH₂)ₙ-L-sm, or nitrogen-protecting group;

R⁶ is CH₂-G¹;

Z^{1′} and Z^{2′} are independently H, or orthogonal hydroxy protecting groups; or one of Z^{1′} and Z^{2′} is H or hydroxy protecting group and the other of Z^{1′} and Z^{2′} is -L-R⁹;

or Z¹′ and Z²′ together with the oxygen atoms they are attached to and the carbon atoms the oxygen atoms are attached to form a ring structure wherein Z^{1′}+Z^{2′} is —C(OAlkyl)(CH₂G¹)-;

G¹, for each occurrence is, independently, H, alkyl, aryl, acetyl, acetonyl, or an electron-withdrawing group;

provided that the compound includes one -L-R⁹.

In a preferred embodiment, the support medium is glass surfaces or particles, including but not limited to, controlled pore glass, succinyl, diglycolyl, hydroquinone-O,O′-diacetate, and oxalyl-derivatized controlled pore glass, silica-containing particles, ceramic particles, quartz particles, polymers, including but not limited to, polystyrene, copolymers of styrene, copolymers of styrene and divinylbenzene, polyethyleneglycols, polyethylene, polypropylene, polyacrylate, polyvinylacetate, polyacrylamides, copolymers of dimethylacrylamide and N,N′-bisacryloylethylenediamine, soluble support media, including but not limited to polyethyleneglycol, or PEPS (polyethylene film grafted with polystyrene chains). More preferred is controlled pore glass, succinyl and diglycolyl controlled pore glass, controlled pore glass derivatized with 1,2-phenylenendioxydiacetic acid and/or 1,4-phenylenendioxydiacetic acid, polystyrene, copolymers of styrene, copolymers of styrene and divinylbenzene, polystyrene grafted with polyethyleneglycol.

In a preferred embodiment, one of Z₁ and Z₂ may be triethylsilyl, triisopropylsilyl, t-butyldimethylsilyl, t-butyldiphenylsilyl, triphenylsilyl, formyl, benzoylformyl, acetyl, methoxyacetyl, phenoxyacetyl, chloroacetyl, dichloroacetyl, trichloroacetyl, trifluoroacetyl, benzoyl, p-phenylbenzoyl, 9-fluorenylmethoxycarbonyl, levulinyl, acetoacetyl groups, or -L-sm, and the other of Z₁ and Z₂ may be H, 4,4′,4″-trimethoxytrityl (TMT), 4,4′-dimethoxytrityl (DMT), 4-methoxytrityl, triphenylmethyl (trityl), 9-phenylxanthen-9-yl (Pixyl), 9-(4-methoxyphenyl)xanthen-9-yl (Mox), 2,7-dimethyl-9-phenylxanthen-9-yl, 2,7-dimethyl-9-(4-methoxyphenyl)xanthen-9-yl, tetrahydropyranyl, 1-ethoxyethyl, 2-trimethylsilylethyl, triethylsilyl, triisopropylsilyl, t-butyldimethylsilyl, t-butydiphenylsilyl, triphenylsilyl, bis(trimethylsilyloxy)cyclooctyloxysilyl, bis(trimethylsilyloxy)cyclododecyloxysilyl, p-phenylazophenyloxycarbonyl (PAPoc), 9-fluorenylmethoxycarbonyl (Fmoc), 2,4-dinitrophenylethoxycarbonyl (DNPEoc), (dialkoxy)alkylmethyl including but not limited to bis(2-acetoxyethoxy)methyl (ACE), levulinyl, or acetoacetyl groups.

In another embodiment, the invention is directed to a method for functionalizing a support medium with a first monomeric subunit, comprising the steps of:

a) Providing a support-bound compound of Formula I;

b) optionally, selectively deblocking one of said orthogonal hydroxy protecting groups Z¹ and Z² to give a reactive hydroxy group or converting said hydroxy protecting group Z¹+Z² to Z¹ and Z² wherein one of Z¹ and Z² is H and the other of Z¹ and Z² is —C(═O)(CH₂G¹); and

c) treating said reactive hydroxy group with a first monomeric subunit having an activated phosphorus group and a further protected hydroxy group thereon for a time and under conditions sufficient to form a monomer-functionalized support medium.

In certain embodiments, the method may further comprise the steps of:

d) treating said monomer-functionalized support medium with a capping agent; and

e) optionally, treating said monomer-functionalized support medium with an oxidizing or sulfurizing agent.

In other embodiments, the method includes the further steps of:

f) deblocking said further protected hydroxy group to give a reactive hydroxy group;

g) treating the reactive hydroxy group with a further monomeric subunit having an activated phosphorus group and a further protected hydroxy group thereon for a time and under conditions sufficient to form an extended compound;

h) treating said extended compound with a capping agent;

i) optionally, treating said extended support-bound compound with an oxidizing or sulfurizing agent;

j) repeating the preceding four steps one or more times to form a further extended compound.

In certain other embodiments, the process further comprises the step of:

k) optionally, selectively deblocking the other of said orthogonal hydroxy protecting groups Z¹ and Z² with a specific deblocking agent to give a reactive hydroxy group; and

l) releasing said oligomeric compound from solid support to solution with a basic reagent effective to cleave said oligomeric compound from said support medium.

Preferably, said selective deblocking step affects no cleavage of phosphate or thiophosphate protecting groups. Preferably, said specific deblocking agent is a solution of hydrazinium or N-methylhydrazinium salt in aqueous or organic media. Preferably, said releasing step is effective to remove protecting groups present on said oligomeric compound. Preferably, said cleaved oligomeric compound has a terminal hydroxy group at the site of cleavage and, more preferably, said terminal hydroxy group is attached to a 2′- or 3′-position of a nucleoside that is located at the 3′-terminus of said oligomeric compound. Preferably, said basic reagent is one of the following:

gaseous ammonia, methylamine, propylamine, or butylamine;

aqueous solution of ammonium hydroxide, methylamine, propylamine, butylamine, ethanolamine, diethanolamine, triethanolamine, lithium hydroxide, sodium hydroxide, potassium hydroxide, lithium carbonate, sodium carbonate, or potassium carbonate;

solution of ammonia, methylamine, propylamine, butylamine, ethanolamine, diethanolamine, triethanolamine, lithium hydroxide, sodium hydroxide, potassium hydroxide, lithium carbonate, sodium carbonate, or potassium carbonate in polar organic solvents;

In a preferred embodiment, the process uses glass surfaces or particles, including but not limited to, controlled pore glass, succinyl, diglycolyl, hydroquinone-O,O′-diacetate, and oxalyl-derivatized controlled pore glass, controlled pore glass derivatized with 1,2-phenylenendioxydiacetic acid and/or 1,4-phenylenendioxydiacetic acid, silica-containing particles, ceramic particles, quartz particles, polymers, including but not limited to, polystyrene, copolymers of styrene, copolymers of styrene and divinylbenzene, polyethyleneglycols, polyethylene, polypropylene, polyacrylate, polyvinylacetate, polyacrylamides, copolymers of dimethylacrylamide and N,N′-bisacryloylethylenediamine, soluble support media, including but not limited to polyethyleneglycol, or PEPS (polyethylene film grafted with polystyrene chains). More preferred is controlled pore glass, succinyl and diglycolyl controlled pore glass, controlled pore glass derivatized with 1,2-phenylenendioxydiacetic acid and/or 1,4-phenylenendioxydiacetic acid, polystyrene, copolymers of styrene, copolymers of styrene and divinylbenzene, polystyrene grafted with polyethyleneglycol.

In a preferred embodiment,the methods of the invention are performed wherein one of Z₁ and Z₂ may be triethylsilyl, triisopropylsilyl, t-butyldimethylsilyl, t-butyldiphenylsilyl, triphenylsilyl, formyl, benzoylformyl, acetyl, methoxyacetyl, phenoxyacetyl, chloroacetyl, dichloroacetyl, trichloroacetyl, trifluoroacetyl, benzoyl, p-phenylbenzoyl, 9-fluorenylmethoxycarbonyl, levulinyl, acetoacetyl groups, or -L-sm, and the other of Z₁ and Z₂ may be H, 4,4′,4″-trimethoxytrityl (TMT), 4,4′-dimethoxytrityl (DMT), 4-methoxytrityl, triphenylmethyl (trityl), 9-phenylxanthen-9-yl (Pixyl), 9-(4-methoxyphenyl)xanthen-9-yl (Mox), 2,7-dimethyl-9-phenylxanthen-9-yl, 2,7-dimethyl-9-(4-methoxyphenyl)xanthen-9-yl, tetrahydropyranyl, 1-ethoxyethyl, 2-trimethylsilylethyl, triethylsilyl, triisopropylsilyl, t-butyldimethylsilyl, t-butydiphenylsilyl, triphenylsilyl, bis(trimethylsilyloxy)cyclooctyloxysilyl, bis(trimethylsilyloxy)cyclododecyloxysilyl, p-phenylazophenyloxycarbonyl (PAPoc), 9-fluorenylmethoxycarbonyl (Fmoc), 2,4-dinitrophenylethoxycarbonyl (DNPEoc), (dialkoxy)alkylmethyl including but not limited to bis(2-acetoxyethoxy)methyl (ACE), levulinyl, or acetoacetyl groups.

Preferably, the linking moiety L is —C(═O)—, —CH₂OC(═O)—, —O—C(═O)—, —P(OR⁷)(═O)—, or —P(OR⁷)(═S)—;

R⁷ is alkyl, cycloalkyl, or —P[O(CH₂)₂CN](═O)—, or —P[O(CH₂)₂CN](═S)—.

Preferably, the treating step of said reactive hydroxy group with a monomeric subunit having an activated phosphorus group and a further protected hydroxy is performed in the presence of an activating agent.

Preferably, said monomeric subunit having an activated phosphorus group is a phosphoramidite, an H-phosphonate or a phosphate triester.

Preferably, one of said groups Z₁ and Z₂ is an acid labile hydroxy protecting group. More preferably, one of said groups Z₁ and Z₂ is hydrogen.

Preferably, each of said further hydroxy protecting groups are acid labile.

In certain preferred embodiments of the process, said hydroxy protecting group Z₁ and each of said further hydroxy protecting groups are removed by contacting said hydroxy protecting groups with an acid, wherein the acid is formic acid, acetic acid, chloroacetic acid, dichloroacetic acid, trichloroacetic acid, trifluoroacetic acid, benzenesulfonic acid, toluenesulfonic acid, or phenylphosphoric acid.

Preferably, the oligomeric compounds may be oligonucleotides, modified oligonucleotides, oligonucleotide analogs, oligonucleosides, oligonucleotide mimetics, short interfering RNA, aptamers, hemimers, gapmers and chimeras. Preferably, said oligomeric compounds include nucleotide chain having from 1 to about 200 monomeric subunits.

### DETAILED DESCRIPTION OF THE INVENTION

The present invention provides compounds and processes useful for the support mediated synthesis of oligomeric compounds. Compounds of the invention are initially attached to support media and subsequently deblocked thereby providing a free hydroxy group. This free hydroxy group is used for oligomer synthesis in an analogous manner to the free 5′-hydroxy group that is provided when using a nucleoside derivatized commercially supplied support medium. In one embodiment, the free hydroxy group of the universal support medium may be reacted with a monomeric subunit having an activated phosphorus group to form a phosphite linkage. The synthesis continues in this manner iteratively until the desired oligomeric compound is prepared. The traditional iterative steps include oxidation, capping and deblocking. When the desired sequence has been iteratively synthesized, the oligomeric compound is released from the support media. In the most preferred embodiment, the release is carried out by treating the solid support-bound oligomeric compound with a base including but not limited to aqueous ammonium hydroxide, aqueous alkylamines, or their mixtures. This removes the group Z², and the released hydroxy group transesterifies the phosphate moiety at the 3′-terminus of said oligomeric compound. so that said oligomeric compound is dephosphorylated at the 3′-terminus and a derivative of ethylene phosphate is formed as a side product.

As used herein, the term “orthogonal protecting groups” refers to functional groups that are protected with different classes of protecting groups, wherein each class of protecting groups can be removed in any order and in the presence of all other classes (see, Barany, G.; Merrifield, R. B. J. Amer. Chem. Soc. 1977, 99, 7363; idem, 1980, 102, 3084). Orthogonal protection is widely used in, for example, automated oligonucleotide synthesis. A functional group is deblocked in the presence of one or more other protected functional groups that are not affected by the deblocking procedure. This deblocked functional group is reacted in some manner and, at some point, a further orthogonal protecting group is removed under a different set of reaction conditions. This allows one to carry out selective chemical transformations to arrive at a desired compound or oligomeric compound.

In the context of this invention, the term “oligomeric compound” refers to a polymeric structure capable of being prepared using well-known support mediated synthetic methods. Preferred oligomeric compounds are also capable of hybridizing a region of a nucleic acid molecule. The term includes oligonucleotides, oligonucleosides, oligonucleotide analogs modified oligonucleotides, oligonucleotide mimetics, hemimers, gapmers and chimeras. Oligomeric compounds can be prepared to be linear or circular and may include branching. They can be prepared single stranded or double stranded and may include overhangs. In general, an oligomeric compound comprises a backbone of linked monomeric subunits where each linked monomeric subunit is directly or indirectly attached to a heterocyclic base moiety. The linkages joining the monomeric subunits, the monomeric subunits and the heterocyclic base moieties can be variable in structure giving rise to a plurality of motifs for the resulting oligomeric compounds, including hemimers, gapmers, and chimeras.

As is known in the art, a nucleoside is a compound consisting of a nucleic base and a sugar moiety. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to the 2′, 3′, or 5′ hydroxy moiety of the sugar. In forming oligonucleotides, the phosphate groups covalently link adjacent nucleosides to form a linear polymeric compound. Within the oligonucleotide structure, the phosphate groups are commonly referred to as forming the internucleoside linkages of the oligonucleotide. The normal internucleoside linkage of RNA and DNA is a 3′ to 5′ phosphodiester linkage.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars, and covalent internucleoside linkages. The terms “oligonucleotide analog” and “modified oligonucleotide” refers to oligonucleotides that have one or more non-naturally occurring portions which function in a similar manner to oligonucleotides. Such modified or substituted oligonucleotides are often preferred over native forms because of desirable properties such as, for example, enhanced cellular uptake, enhanced affinity for nucleic acid target and increased stability in the presence of nucleases.

In the context of this invention, the term “oligonucleoside” refers to nucleosides that are joined by internucleoside linkages not having phosphorus atoms. Internucleoside linkages of this type include short chain alkyl, cycloakyl, mixed heteroatom alkyl, mixed heteroatom cycloalkyl, one or more short chain heteroatomic and one or more short chain heterocyclic linkages. These internucleoside linkages include but are not limited to siloxane, sulfide, sulfoxide, sulfone, acetyl, formacetyl, thioformacetyl, methylene formacetyl, thioformacetyl, alkenyl, sulfamate; methyleneimino, methylenehydrazino, sulfonate, sulfonamide, amide, and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,608,046; 5,610,289, 5,618,704; 5,623,070; 5,633,360; 5,646,269; 5,663,312; 5,677,437;5,677,439; and 5,792,608 each of which is herein incorporated by reference.

One interesting class of oligonucleotide is short interfering RNA (siRNA). RNA interference is an evolutionarily conserved process for control of gene expression. In the process of gene silencing by RNAi, double-stranded RNA (dsRNA) causes specific degradation of mRNAs that are homologous in sequence to the RNAi. The mechanism of gene silencing by short, dsRNAs was first observed in C. elegans. The siRNA responsible for RNA interference were first isolated from plants Subsequently, siRNAs have been observed in Drosophila and synthetic RNAi has been shown to silence genes in a host of systems including cultured mammalian cells (for recent reviews see Tuschl, T. “RNA Interference and Small Interfering RNAs.” ChemBioChem 2001 2, 239-245.; McManus M T, Sharp P A “Gene Silencing in Mammals by Small Interfering RNAs.” Nature Reviews Genetics 2002 3, 737-747; Manoharan M “RNA interference and chemically modified siRNAs.” Nucleic Acids Res Suppl 2003 3 115-6).

Within cells, siRNAs are incorporated into an RNA-induced silencing complex (RISC), forming a stable protein-RNA complex. The RISC complex, activated by ATP, unwinds the duplex formed by the siRNA strands. One of the strands of the siRNA duplex is complementary to the mRNA target and guides the endonucleolytic cleavage of the target mRNA. Thus, the high specificity of the technology results from the Watson-Crick base pairing the RNAi oligonucleotide with the target mRNA.

It is hypothesized that the underlying mechanism of RNA interference evolved from a natural defense mechanism against viruses. Unlike the cells of simpler organisms, such as plants and insects, mammalian cells react to the presence of long strands of dsRNA by triggering cellular suicide with the simultaneous release of interferon to warn neighboring cells of a viral invasion. However, researchers have now shown that RNA interference can result in mammalian cells when the RNAi is introduced as short duplexes of 21-25 base pairs derived from synthetic RNA duplexes(Elbashir S M, et al. “Duplexes of 21-nucleotide RNAs mediate RNA interference in mammalian cell culture.” Nature 2001, 411, 494-498).

In the context of this invention, the term “oligonucleotide mimetic” refers to an oligonucleotide wherein the backbone of the nucleotide units has been replaced with novel groups. Although the term is intended to include oligomeric compounds wherein only the furanose ring or both the furanose ring and the internucleotide linkage are replaced with novel groups, replacement of only the furanose ring is also referred and the internucleotide linkage are replaced with novel groups, replacement of only the furanose ring is also referred to in the art as being a sugar surrogate. Oligonucleotide mimetics can be further modified to incorporate one or more modified heterocyclic base moieties to enhance properties such as hybridization.

The internucleotide linkage found in native nucleic acids is a phosphodiester linkage. This linkage has not been the linkage of choice for synthetic oligonucleotides that are for the most part targeted to a portion of a nucleic acid such as mRNA because of their rapid degradation by nucleases. Preferred internucleotide linkages and internucleoside linkages as is the case for non-phosphate ester type linkages include, for example, phosphorothioates, chiral phosphorothioates, phosphorodithioates, phosphotriesters, aminoalkylphosphotriesters, methyl and other alkyl phosphonates, phosphoramidates.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821, 5541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is hrein incorporated by reference.

Oligomeric compounds can have a variety of substituent groups attached at various positions. Furanosyl groups found in native nucleic acids as well as various oligomeric compounds can be substituted at a number of positions. The most frequently substituted position is the 2′-position of ribose. The 3′, 4′, and 5′ have also been substituted with substituent groups referred to as sugar substituent groups. Preferred sugar substituent groups include: OH; F; O—, S—, or N-alkyl, wherein the alkyl may be substituted or unsubstituted C, to C₁₀ alkyl. Other sugar substituent groups include: RNA cleaving groups, reporter groups, intercalators, groups for improving the pharmacokinetic properties of oligonucleotides, or groups for improving the pharmacodynamic properties of oligonucleotides.

Oligomeric compounds may also include nucleic base (often referred to in the art simply as “base” or “heterocyclic base moiety”) modifications or substitutions. As used herein, “unmodified” or “natural” nucleobases include the purine bases adenine (A) and guanine (G), and the pyrimidine baes thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 8-substitued adenines and guanines, 5-substituted uracils and cytosines, 7-methylguanine, and 7-methyladenine.

Chimeric oligomeric compounds of the invention may be formed as composite structures of two or more oligonucleotides, modified oligonucleotides, oligonucleotide analogs, oligonucleosides, and/or oligonucleotides mimetics as described above. Such compounds have also been referred to in the art as hybrids hemimers, gapmers or inverted gapmers.

The reagents useful in the synthesis of oligomeric compounds have the structure of Formula I:

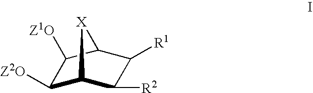

wherein:

X is O or NR³;

R³ is -L-sm, alkyl, —C(═O)alkyl, —C(═O)aryl, —C(═O)NH-alkyl, —C(═O)NH-aryl or an amino protecting group;

L is a linking moiety;

sm is a support medium;

R¹ and R² are independently H, alkyl, —C(═O)—R⁴; or R¹ and R² are fused to form a ring structure so that R¹+R² is —C(═O)—N(R⁵)—C(═O)—; or R¹ and R² together with the carbon atoms they are attached to form a substituted or unsubstitute cycloalkyl, a substituted or unsubstituted aryl, a substituted or unsubstituted heterocycloalkyl, or a substituted or unsubstituted heteroaryl; or one of R₁ and R₂ is -L-sm and the other of R¹ and R² is H, O—C(═O)R⁶, or —C(═O)—R⁴;

R⁴ is —O(alkyl), —O(benzyl), —O(alkoxyalkyl), or —N(J¹)J²;

J¹ is H or alkyl;

J² is H, alkyl, benzyl, alkoxyalkyl, —(CH₂)ₙ—O-L-sm, or a nitrogen-protecting group;

n is an integer from 0 to about 12;

or J¹ and J² together with the nitrogen atom they are attached to form a heteroaryl or heterocycloalkyl;

R⁵ is alkyl, aryl, benzyl, alkoxyalkyl, —(CH₂)ₙ-L-sm, or nitrogen-protecting group;

R⁶ is CH₂-G¹;

Z¹ and Z² are independently H, or orthogonal hydroxy protecting groups; or one of Z¹ or Z² is H and the other of Z¹ or Z² is —C(═O)CH₂G¹; or one of Z¹ or Z² is H or hydroxy protecting group and the other of Z¹ or Z² is -L-sm;

or Z₁ and Z₂ together with the oxygen atoms they are attached to and the carbon atoms the oxygen atoms are attached to form a ring structure wherein Z¹+Z² is —C(OAlkyl)(CH₂G¹)-;

G¹, for each occurrence, is independently, H, alkyl, aryl, acetyl, acetonyl, or an electron-withdrawing group;

provided that when one of R¹ or R² is -L-sm and the other of R¹ and R² is O—C(═O)R⁶ or —C(═O)—R⁴, then Z¹ and Z² together with the oxygen atoms they are attached to and the carbon atoms the oxygen atoms are attached to form a ring structure wherein Z¹+Z² is —C(OAlkyl)(CH₂G¹)-; and provided that the compound includes one -L-sm.

Preferably, L is —C(═O)—, —CH₂OC(═O)—, —O—C(═O)—, —P(OR⁷)(═O)—, or —P(OR⁷)(═S)—; R⁷ is alkyl, cycloalkyl, or —P[O(CH₂)₂CN](═O)—, or —P[O(CH₂)₂CN](═S)—.

In one preferred embodiment, the invention is directed to compounds of Formula II:

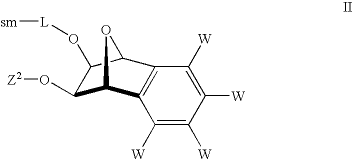

wherein:

L and sm are defined as above;

Z² is 4,4′-dimethoxytrityl group, 4,4′,4″-trimethoxytrityl group, or H; and W, for each occurrence, is independently H or a halogen atom.

Preferably, W is a halogen atom. More preferably yet, W is F. More preferably, L is —C(═O)—.

In another preferred embodiment, the invention is directed to compounds of Formula III:

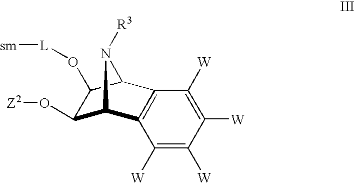

wherein:

L and sm are defined as above;

R³ is alkyl, —C(═O)alkyl, —C(═O)NH(Alkyl), —C(═O)NH(Aryl) or a nitrogen protecting group;

Z² is 4,4′-dimethoxytrityl group, 4,4′,4″-trimethoxytrityl group, or H; and W, for each occurrence, is independently H or a halogen atom.

Preferably, W is a halogen atom. More preferably yet, W is F. More preferably, L is —C(═O)—.

In yet another preferred embodiment, the invention is directed to compounds having one of Formulas IVa and IVb:

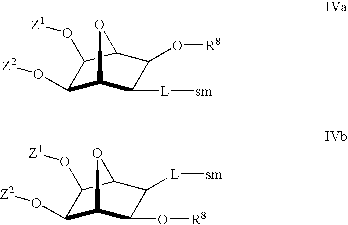

wherein:

L and sm are defined as above;

R⁸ is —C(═O)CH₂-G¹

one of Z¹ and Z² is —C(═O)CH₂-G¹ and the other of Z¹ and Z² is 4,4′-dimethoxytrityl group, 4,4′,4″-trimethoxytrityl group, or H; or Z¹ and Z² together with the oxygen atoms they are attached to and the carbon atoms the oxygen atoms are attached to form a ring structure so that Z¹+Z² is —C(OAlkyl)(CH₂G¹)-.

Preferably, L is —C(═O)—. Preferably, G¹ is H, Cl, acetyl, acetonyl, OCH₃, or —OC₆H₅.

In a more preferred embodiment, the invention is directed to compounds having one of Formulas Va and Vb:

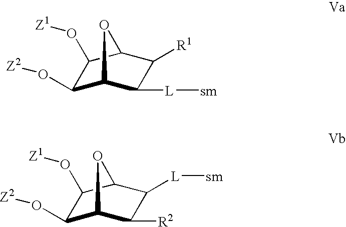

wherein:

L and sm are defined as above;

R¹ and R² are each, independently, H or —C(═O)—R⁴;

one of Z¹ or Z² is H and the other of Z¹ or Z² is —C(═O)CH₂G¹; or Z¹ and Z² together with the oxygen atoms they are attached to and the carbon atoms the oxygen atoms are attached to form a ring structure so that Z¹+Z² is —C(OAlkyl)(CH₂G¹)-.

Preferably, L is —C(═O)—, —CH₂OC(═O)—, —O—C(═O)—, —P(OR⁷)(═O)—, or —P(OR⁷)(═S)—; R⁷ is alkyl, cycloalkyl, or —P[O(CH₂)₂CN](═O)—, or —P[O(CH₂)₂CN](═S)—.

Preferably, G¹ is H, acetyl, acetonyl, Cl, OCH₃, or —OC₆H₅.

In a more preferred embodiment, the invention is directed to compounds having one of Formulas VIa and VIb:

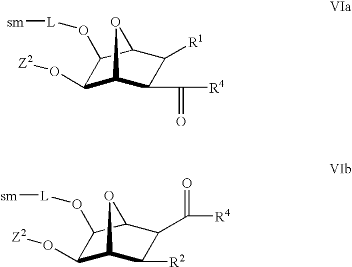

wherein:

L and sm are defined as above;

R¹ and R² are each, independently, H or —C(═O)—R⁴; and

Z² is 4,4′-dimethoxytrityl group, 4,4′,4″-trimethoxytrityl group, or H.

Preferably, L is —C(═O)—.

In an even more preferred embodiment, the invention is directed to compounds of Formula VII:

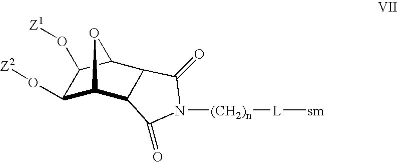

wherein:

sm is defined as above;

L is —OC(═O)—, —C(═O)— or —OP(OR⁷)(═Y)—;

R⁷ is a negative charge, alkyl, cycloalkyl, or phosphate protecting group;

Y is O or S; and

one of Z¹ and Z² is —C(═O)CH₂-G¹ where G¹ is H, an alkyl group, or an electron-withdrawing group and the other of Z¹ and Z² is 4,4′-dimethoxytrityl group, 4,4′,4″-trimethoxytrityl group, or H; or Z¹ and Z² together with the oxygen atoms they are attached to and the carbon atoms the oxygen atoms are attached to form a ring structure so that Z¹+Z² is —C(OAlkyl)(CH₂G¹)-.

Preferably, G¹ is H, Cl, acetyl, acetonyl, OCH₃, or —OC₆H₅.

In another even more preferred embodiment, the invention is directed to compounds of Formula VIII:

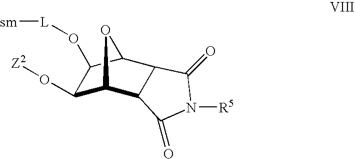

wherein:

L, sm and R⁵ are defined as above;

Z² is 4,4′-dimethoxytrityl group, 4,4′,4″-trimethoxytrityl group, or H.

Preferably, R⁵ is methyl, ethyl, propyl, iso-propyl, phenyl, or benzyl group. Preferably, L is —C(═O)—.

In another embodiment, the invention is directed to compounds of formula Ia:

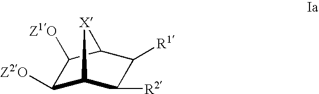

wherein:

X′ is O or NR^{3′};

R^{3′} is -L-R⁹, alkyl, —C(═O)alkyl, —C(═O)aryl, —C(═O)NH-alkyl, —C(═O)NH-aryl or an amino protecting group;

L is a linking moiety;

R⁹ is —X²—P(X₃R⁷)NJ³J⁴;

X² and X³ are each, independently, O or S;

R⁷ is a negative charge, alkyl, cycloalkyl or phosphate protecting group;

J³ and J⁴ are each, independently, and alkyl, a cycloalkyl, or an arylalkyl, or J³ and J⁴ together with the nitrogen atom they are attached to form a heteroaryl or heterocycloalkyl;

R^{1′} and R^{2′} are independently H, alkyl, —C(═O)—R⁴; or R^{1′} and R^{2′} are fused to form a ring structure so that R^{1′}+R^{2′} is —C(═O)—N(R⁵)—C(═O)—; or R^{1′} and R^{2′} together with the carbon atoms they are attached to form a substituted or unsubstitute cycloalkyl, a substituted or unsubstituted aryl, a substituted or unsubstituted heterocycloalkyl, or a substituted or unsubstituted heteroaryl; or one of R^{1′} and R^{2′} is -L-R⁹ and the other of R¹ and R² is H, O—C(═O)R⁶, or —C(═O)—R⁴;

R⁴ is —O(alkyl), —O(benzyl), —O(alkoxyalkyl), or —N(J¹)J²;

J¹ is H or alkyl;

J² is H, alkyl, benzyl, alkoxyalkyl, —(CH₂)ₙ—O-L-sm, or a nitrogen-protecting group;

n is an integer from 0 to about 12;

or J¹ and J² together with the nitrogen atom they are attached to form a heteroaryl or heterocycloalkyl;

R⁵ is alkyl, aryl, benzyl, alkoxyalkyl, —(CH₂)ₙ-L-sm, or nitrogen-protecting group;

R⁶ is CH₂-G¹;

Z^{1′} and Z^{2′} are independently H, or orthogonal hydroxy protecting groups; or one of Z^{1′} and Z^{2′} is H or hydroxy protecting group and the other of Z^{1′} and Z^{2′} is -L-R⁹;

or Z¹′ and Z²′ together with the oxygen atoms they are attached to and the carbon atoms the oxygen atoms are attached to form a ring structure wherein Z^{1′}+Z^{2′} is —C(OAlkyl)(CH₂G¹)-;

G¹, for each occurrence is, independently, H, alkyl, aryl, acetyl, acetonyl, or an electron-withdrawing group;

provided that the compound includes one -L-R⁹.

To prepare compounds of the invention wherein X is O or NR₃, one may begin with starting materials known to those skilled in the art. The synthesis of 5,6,7,8-tetrafluoro-1,4-dihydro-1,4-epoxynaphthalene and 5,6,7,8-tetrafluoro-1,4-dihydro-9-methylnaphthalen-1,4-imine has been disclosed in Caster, K. C.; Keck, C. G.; Walls, R. D. J. Org. Chem. 2001, 66, 2932-2936; Gribble, G. W.; LeHoullier, C. S.; Sibi, M. P.; Allen, R. W. J. Org. Chem. 1985, 50, 1611-16; and Priestley, G. M.; Warrener, R. N. Tetrahedron Lett. 1972, 42, 4295-8. The preparation of exo- and endo-isomers of 7-oxabicyclo[2.2.1]hept-4-ene-2,3-dicarboximide has been disclosed in Kwart, H.; Burchuk, I. J. Amer. Chem. Soc. 1952, 74, 3094-3097. Similarly, N-phenyl-7-oxabicyclo[2.2.1]hept-5-ene-2,3-dicarboximide 41 can be readily synthesized by Diels-Alder reaction as disclosed in Cooley, J. H.; Williams, R. V. J. Chem. Education 1997, 74, 582-585. Exo-7-oxabicyclo[2.2.1]hept-5-ene-2,3-dicarboxylic anhydride is commercially available from a number of suppliers and can be readily accessed by well-established procedures (Woodward, R. B.; Baer, Harold. J. Amer. Chem. Soc. 1948, 70, 1161-6 Seltzer, S. J. Amer. Chem. Soc. 1965, 87, 1534-40). Following the previously disclosed method, the latter compound can be readily bis-hydroxylated to give the compound 2 (Daniels, R.; Fischer, J. L. J. Org. Chem. 1963, 28, 320-2.

In a preferred embodiment, the support medium is glass surfaces or particles, including but not limited to, controlled pore glass, succinyl, diglycolyl, hydroquinone-O,O′-diacetate, and oxalyl-derivatized controlled pore glass, controlled pore glass derivatized with 1,2-phenylenendioxydiacetic acid and/or 1,4-phenylenendioxydiacetic acid, silica-containing particles, ceramic particles, quartz particles, polymers, including but not limited to, polystyrene, copolymers of styrene, copolymers of styrene and divinylbenzene, polyethyleneglycols, polyethylene, polypropylene, polyacrylate, polyvinylacetate, polyacrylamides, copolymers of dimethylacrylamide and N,N′-bisacryloylethylenediamine, soluble support media, including but not limited to polyethyleneglycol, or PEPS (polyethylene film grafted with polystyrene chains). More preferred is controlled pore glass, succinyl and diglycolyl controlled pore glass, controlled pore glass derivatized with 1,2-phenylenendioxydiacetic acid and/or 1,4-phenylenendioxydiacetic acid, polystyrene, copolymers of styrene, copolymers of styrene and divinylbenzene, polystyrene grafted with polyethyleneglycol.

In a preferred embodiment, one of Z₁ and Z₂ may be triethylsilyl, triisopropylsilyl, t-butyldimethylsilyl, t-butyldiphenylsilyl, triphenylsilyl, formyl, benzoylformyl, acetyl, methoxyacetyl, phenoxyacetyl, chloroacetyl, dichloroacetyl, trichloroacetyl, trifluoroacetyl, benzoyl, p-phenylbenzoyl, 9-fluorenylmethoxycarbonyl, levulinyl, acetoacetyl groups, or -L-sm, and the other of Z₁ and Z₂ may be H, 4,4′,4″-trimethoxytrityl (TMT), 4,4′-dimethoxytrityl (DMT), 4-methoxytrityl, triphenylmethyl(trityl), 9-phenylxanthen-9-yl (Pixyl), 9-(4-methoxyphenyl)xanthen-9-yl (Mox), 2,7-dimethyl-9-phenylxanthen-9-yl, 2,7-dimethyl-9-(4-methoxyphenyl)xanthen-9-yl, tetrahydropyranyl, 1-ethoxyethyl, 2-trimethylsilylethyl, triethylsilyl, triisopropylsilyl, t-butyldimethylsilyl, t-butydiphenylsilyl, triphenylsilyl, bis(trimethylsilyloxy)cyclooctyloxysilyl, bis(trimethylsilyloxy)cyclododecyloxysilyl, p-phenylazophenyloxycarbonyl (PAPoc), 9-fluorenylmethoxycarbonyl (Fmoc), 2,4-dinitrophenylethoxycarbonyl (DNPEoc), (dialkoxy)alkylmethyl including but not limited to bis(2-acetoxyethoxy)methyl (ACE), levulinyl, or acetoacetyl groups.

Other representative hydroxy protecting groups commonly used in the art may be found in Beaucage, et al., Tetrahedron 1992, 48, 2223; and Greene and Wuts, Protective Groups in Organic Synthesis, Chapter 2, 2nd ed., John Wiley & Sons, New York, 1991, each of which are hereby incorporated by reference in their entirety. Preferred protecting groups include dimethoxytrityl (DMT), monomethoxytrityl, 9-phenylxantehn-9-yl (Pixyl) and 9-(p-methoxyphenyl)xanthen-9-yl (Mox).

Further representative protecting groups utilized in oligonucleotide synthesis are discussed in Agrawal, et al., Protocols for Oligonucleotide Conjugates, Eds, Humana Press; New Jersey, 1994; Vol. 26 pp. 1-72.

The methods of the invention are useful for functionalizing a support medium with a first monomeric subunit. In one embodiment, the method comprises the steps of:

a) Providing a support-bound compound of Formula I:

b) optionally, selectively deblocking one of said orthogonal hydroxy protecting groups Z¹ and Z² to give a reactive hydroxy group or converting said hydroxy protecting group Z¹+Z² to Z¹ and Z² wherein one of Z¹ and Z² is H and the other of Z¹ and Z² is —C(═O)(CH₂G¹); and

c) treating said reactive hydroxy group with a first monomeric subunit having an activated phosphorus group and a further protected hydroxy group thereon for a time and under conditions sufficient to form a monomer-functionalized support medium.

In certain embodiments, the method may further comprise the steps of:

d) treating said monomer-functionalized support medium with a capping agent; and

e) optionally, treating said monomer-functionalized support medium with an oxidizing or sulfurizing agent.

In other embodiments, the method includes the further steps of:

f) deblocking said further protected hydroxy group to give a reactive hydroxy group;

g) treating the reactive hydroxy group with a further monomeric subunit having an activated phosphorus group and a further protected hydroxy group thereon for a time and under conditions sufficient to form an extended compound;

h) treating said extended compound with a capping agent;

i) optionally, treating said extended support-bound compound with an oxidizing or sulfurizing agent;

j) repeating the preceding four steps one or more times to form a further extended compound.

In certain other embodiments, the process further comprises the step of:

k) optionally, selectively deblocking the other of said orthogonal hydroxy protecting groups Z¹ and Z² with a specific deblocking agent to give a reactive hydroxy group; and

l) releasing said oligomeric compound from solid support to solution with a basic reagent effective to cleave said oligomeric compound from said support medium.

Preferably, said selective deblocking step affects no cleavage of phosphate or thiophosphate protecting groups. Preferably, said specific deblocking agent is a solution of hydrazinium or N-methylhydrazinium salt in aqueous or organic media. Preferably, said releasing step is effective to remove protecting groups present on said oligomeric compound. Preferably, said cleaved oligomeric compound has a terminal hydroxy group at the site of cleavage and, more preferably, said terminal hydroxy group is attached to a 2′- or 3′-position of a nucleoside that is located at the 3′-terminus of said oligomeric compound. Preferably, said basic reagent is one of the following:

gaseous ammonia, methylamine, propylamine, or butylamine;

aqueous solution of ammonium hydroxide, methylamine, propylamine, butylamine, ethanolamine, diethanolamine, triethanolamine, lithium hydroxide, sodium hydroxide, potassium hydroxide, lithium carbonate, sodium carbonate, or potassium carbonate;

solution of ammonia, methylamine, propylamine, butylamine, ethanolamine, diethanolamine, triethanolamine, lithium hydroxide, sodium hydroxide, potassium hydroxide, lithium carbonate, sodium carbonate, or potassium carbonate in polar organic solvents;

In a preferred embodiment, the process uses glass surfaces or particles, including but not limited to, controlled pore glass, succinyl, diglycolyl, hydroquinone-O,O′-diacetate, and oxalyl-derivatized controlled pore glass, controlled pore glass derivatized with 1,2-phenylenendioxydiacetic acid and/or 1,4-phenylenendioxydiacetic acid, silica-containing particles, ceramic particles, quartz particles, polymers, including but not limited to, polystyrene, copolymers of styrene, copolymers of styrene and divinylbenzene, polyethyleneglycols, polyethylene, polypropylene, polyacrylate, polyvinylacetate, polyacrylamides, copolymers of dimethylacrylamide and N,N′-bisacryloylethylenediamine, soluble support media, including but not limited to polyethyleneglycol, or PEPS (polyethylene film grafted with polystyrene chains). More preferred is controlled pore glass, succinyl and diglycolyl controlled pore glass, controlled pore glass derivatized with 1,2-phenylenendioxydiacetic acid and/or 1,4-phenylenendioxydiacetic acid, polystyrene, copolymers of styrene, copolymers of styrene and divinylbenzene, polystyrene grafted with polyethyleneglycol.

In a preferred embodiment,the methods of the invention are performed wherein one of Z₁ and Z₂ may be triethylsilyl, triisopropylsilyl, t-butyldimethylsilyl, t-butyldiphenylsilyl, triphenylsilyl, formyl, benzoylformyl, acetyl, methoxyacetyl, phenoxyacetyl, chloroacetyl, dichloroacetyl, trichloroacetyl, trifluoroacetyl, benzoyl, p-phenylbenzoyl, 9-fluorenylmethoxycarbonyl, levulinyl, acetoacetyl groups, or -L-sm, and the other of Z₁ and Z₂ may be H, 4,4′,4″-trimethoxytrityl (TMT), 4,4′-dimethoxytrityl (DMT), 4-methoxytrityl, triphenylmethyl (trityl), 9-phenylxanthen-9-yl (Pixyl), 9-(4-methoxyphenyl)xanthen-9-yl (Mox), 2,7-dimethyl-9-phenylxanthen-9-yl, 2,7-dimethyl-9-(4-methoxyphenyl)xanthen-9-yl, tetrahydropyranyl, 1-ethoxyethyl, 2-trimethylsilylethyl, triethylsilyl, triisopropylsilyl, t-butyldimethylsilyl, t-butydiphenylsilyl, triphenylsilyl, bis(trimethylsilyloxy)cyclooctyloxysilyl, bis(trimethylsilyloxy)cyclododecyloxysilyl, p-phenylazophenyloxycarbonyl (PAPoc), 9-fluorenylmethoxycarbonyl (Fmoc), 2,4-dinitrophenylethoxycarbonyl (DNPEoc), (dialkoxy)alkylmethyl including but not limited to bis(2-acetoxyethoxy)methyl (ACE), levulinyl, or acetoacetyl groups.

Preferably, the linking moiety L is —C(═O)—, —CH₂OC(═O)—, —O—C(═O)—, —P(OR⁷)(═O)—, or —P(OR⁷)(═S)—;

R⁷ is alkyl, cycloalkyl, or —P[O(CH₂)₂CN](═O)—, or —P[O(CH₂)₂CN](═S)—.

Preferably, the treating step of said reactive hydroxy group with a monomeric subunit having an activated phosphorus group and a further protected hydroxy is performed in the presence of an activating agent.

Preferably, said monomeric subunit having an activated phosphorus group is a phosphoramidite, an H-phosphonate or a phosphate triester.

Preferably, one of said groups Z₁ and Z₂ is an acid labile hydroxy protecting group. More preferably, one of said groups Z₁ and Z₂ is hydrogen.

Preferably, each of said further hydroxy protecting groups are acid labile.

In certain preferred embodiments of the process, said hydroxy protecting group Z₁ and each of said further hydroxy protecting groups are removed by contacting said hydroxy protecting groups with an acid, wherein the acid is formic acid, acetic acid, chloroacetic acid, dichloroacetic acid, trichloroacetic acid, trifluoroacetic acid, benzenesulfonic acid, toluenesulfonic acid, or phenylphosphoric acid.

Preferably, the oligomeric compounds may be oligonucleotides, modified oligonucleotides, oligonucleotide analogs, oligonucleosides, oligonucleotide mimetics, short interfering RNA, aptamers, hemimers, gapmers and chimeras. Preferably, said oligomeric compounds include nucleotide chain having from 1 to about 200 monomeric subunits.

The hydroxy-protecting group can be removed from the compounds of the invention by techniques well known in the art to form the free hydroxy. For example, dimethoxytrityl protecting groups can be removed by protic acids such as formic acid, dichloroacetic acid, trichloroacetic acid, p-toluene sulfonic acid or with Lewis acids such as for example zinc bromide. See, for example, Greene, T. W. and Wuts, P. G. M. Protective groups in organic synthesis. 3^{rd} Ed. Wiley & Sons: New York, 1999.

The oligomeric compounds prepared in accordance with the process of the invention may be conveniently and routinely made through the well-known technique of support-based synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. Suitable solid phase techniques, including automated synthesis techniques, are described in F. Eckstein (ed.), Oligonucleotides and Analogues, A Practical Approach, Oxford University Press, New York ( 1991).

Preferably, the oligomeric compounds prepared by the process of the invention utilize phosphoramidite chemistry on the support medium. The phosphoramidites can be modified at the heterocyclic base, the sugar or both positions to enable the synthesis of fully modified positionally modified oligonucleotides and their analogs.

Conventional iterative solid phase oligonucleotide synthetic regimes are utilized to synthesize the oligomeric compounds of the invention. Representative support-based techniques are those typically employed for DNA and RNA synthesis utilizing standard phosphoramidite chemistry, (see, e.g., Protocols for Oligonucleotides And Analogs, Agrawal, S., ed., Humana Press, Totowa, N.J., 1993, hereby incorporated by reference in its entirety). Further details of methods useful for preparing oligonucleotides may be found in Sekine, M., et al., J. Org. Chem., 1979, 44, 2325; Dahl, O., Sulfur Reports, 1991, 11, 167-192; Kresse, ., et. al., Nucleic Acids Research, 1975, 2, 1-9; Eckstein, F., Ann. Rev. Biochem., 1985, 54, 367-402; and U.S. Pat. No. 5,210,264.

A preferred synthetic solid phase synthesis of oligonucleotides utilized phosphoramidites as activated phosphate compounds. In this technique, a phosphoramidite monomer is reacted with a free hydroxy on the growing oligomer chain to produce an intermediate phosphite compound, which is subsequently oxidized to the P(V) state using standard methods. This technique is commonly used for the synthesis of several types of linkages including phosphodiester, phosphorothioate, and phosphorodithioate linkages.

The phosphite triester linkage is subsequently oxidized or sulfurized. Choice of oxidizing or sulfurizing agent will determine whether the linkage will be oxidized or sulfurized to a phosphotriester or thiophosphotriester.

It is generally preferable to perform a capping step, either prior to or after oxidation or sulfurization of the phosphite triester, thiophosphite triester, or dithiophosphite triester. Such a capping step is generally known to be beneficial by blocking chains that have not reacted in the coupling cycle and thus preventing their further elongation. One representative reagent used for capping is acetic anhydride. Other suitable capping reagents and methodologies can be found in U.S. Pat. No. 4,816,571, hereby incorporated by reference in its entirety. Treatment with an acid removes the 5′-hydroxy protecting group, and the synthetic cycle is repeated until the desired oligomer is assembled.

A representative list of capping reagents useful in the process of the present invention include without limitation, acetic anhydride, t-butylphenoxyacetic anhydride, phosphite monoesters, and selected acid chlorides preferably delivered concurrently with a nucleophilic catalyst and a strong base such as for example dimethylaminopyridine, N-methylimidazole or triethylamine. Generally, capping reagents comprise a mixture of Cap A and Cap B.

Representative mixtures include without limitation:

Cap A: acetic anhydride in acetonitrile or tetrahydrofuran; chloroacetic anhydride in acetonitrile or tetrahydrofuran;

Cap B:N-methylimidazole and pyridine in acetonitrile or tetrahydrofuran; 4-dimethylaminopyridne (DMAP) and pyridine in acetonitrile or tetrahydrofuran; 2,6-litidine and N-methylimidazole in acetonitrile or tetrahydrofuran.

A more detailed description capping reagents is discussed in U.S. Pat. No. 4,816,571, issued Mar. 28, 1989, which is incorporated herein by reference. A preferred capping reagent is acetic anhydride routinely used as a mixture of cap A and cap B.

Useful sulfurizing agents include Beaucage reagent described in e.g., Iyer et al., J. Amer. Chem. Soc. 1990, 112, 1253-1254; and Iyer et al., J Org Chem. 1990, 55, 4693-4699; tetraethylthiuram disulfide as described in Vu et al., Tetrahedron Lett., 1991, 32, 3005-3007; dibenzoyl tetrasulfide as described in Rao et al., Tetrahedron Lett. 1992, 33, 4829-4842; di(phenylacetyl)disulfide, as described in Kamer, et al., Tetrahedron Lett. 1989, 30, 6757-6760; bis(O,O-diisopropoxy phosphinothioyl)disulfide, Wojceich J. Stec., Tetrahedron Lett. 1993, 34, 5317-5320; sulfur; and sulfur in combination with ligands like triaryl, trialkyl or triaralkyl or trialkaryl phophines. Useful oxidizing agents, in addition to those set out above, include iodine/tetrahydrofuran/water/pyridine; hydrogen peroxide/water; tert-butyl hydroperoxide; or m-chloroperbenzoic acid. In case of sulfurization, the reaction is performed under anhydrous conditions with the exclusion of oxygen; whereas, in the case of oxidation the reaction can be performed under aqueous conditions.

The internucleoside linkages of the oligonucleotides described herein, can be any internucleoside linkage as is known in the art, including phosphorus based linking groups, such as phosphite, phosphodiester, phosphorothioate, and phosphorodithioate linkages. Such linkages can be protected, i.e., they can bear, for example, phosphate-protecting groups. As used herein, the term “phosphorus protecting group” is intended to denote protecting groups that are known to be useful to protect phosphorus-containing linkages during oligonucleotide synthesis. One such preferred phosphorus-protecting group is the 2-cyanoethyl protecting group.

The processes of the present invention illustrate the use of activated phosphorus compounds (e.g., compounds having activated phosphorus-containing substituent groups) in coupling reactions. As used herein, the term “activated phosphorus compounds” includes monomers and oligomers that have an activated phophorus-containing substituent group that is reactive with a hydroxy group of another monomeric or oligomeric compound to form a phophours-containing internucleotide linkage. Such activated phosphorus groups contain activated phosphorus atoms in p^{III} valence state and are known in the art and include, but are not limited to, phosphoramidite, H-phosphonate, phosphate trimesters and chiral auxiliaries. A preferred synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in a preferred embodiment, phosphodiester or phosphorothioate internucleotide linkages. Additional activated phosphates and phosphates are disclosed in Beaucage and Iyer, Tetrahedron 1992, 48, 2223-2311.

Activated phosphorus groups are useful in the preaparation of a wide range of oligomeric compounds including but not limited to oligonucleosides and oligonucleotides as well as oligonucleotides that have been modified or conjugated with other groups at the base or sugar or both. A representative example of one type of oligomer synthesis that utilizes the coupling of an activated phosphorus group with a reactive hydroxy group is the widely used phosphoramidite approach. A phosphoramidite monomeric subunit is reacted under appropriate conditions with a reactive hydroxy group to form a phosphite linkage that is further oxidized to a phosphodiester or phosphorothioate linkage. This approach commonly utilized nucleoside phosphoramidites of the formula:

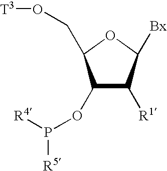

wherein

each Bx′ is an optionally protected heterocyclic base moiety;

each R^{1′} is, independently, H or an optionally protected sugar substituent group;

T^{3′} is a hydroxy protecting group, a nucleoside, a nucleotide, an oligonucleoside or an oligonucleotide;

R^{4′} is N(L¹)L²;

each L¹ and L² is, independently, C₁₋₆ alkyl;

or L¹ and L² are joined together to form a 4 to 7-membered heterocyclic ring system including the nitrogen atom to which L¹ and L² are attached, wherein said ring system optionally includes at least one additional heteroatom, wherein said heteroatom is O, N or S;

R⁵ is Pg-O—, Pg-S—, C₁₋₁₀ alkyl, CH₃(CH₂)₀₋₁₀—O— or —NR⁶R⁷;

Pg is a protecting/blocking group; and

each R⁶ and R⁷ is, independently, hydrogen, C₁₋₁₀ alkyl, cycloalkyl or aryl;

or optionally, R⁶ and R⁷, together with the nitrogen atom to which they are attached form a cyclic moiety that may include an additional heteroatom, wherein said heteroatom is O, S and N; or

R⁴ and R⁵ together with the phosphorus atom to which R⁴ and R⁵ are attached form a chiral auxiliary.

Some representative examples of R⁴ groups that are known to those skilled in the art and are amenable to the present invention are N(CH₃)₂, N(C₂H₅)₂, N(i-C₃H₇)₂, pyrrolydino, or morpholino.

Some representative examples of R⁵ groups that are known to those skilled in the art and are amenable to the present invention are O(CH₂)₂CN, OCH₃, O(CH₂)CH═CH₂.

Representative hydroxy protecting groups commonly used in the art may be found in Beaucage, et al., Tetrahedron 1992, 48, 2223; and Greene and Wuts, Protective Groups in Organic Synthesis, Chapter 2, 2d ed, John Wiley & Sons, New York, (1991), each of which are hereby incorporated by reference in their entirety. Preferred protecting groups include trimethoxytrityl, dimethoxytrityl (DMT), monomethoxytrityl, 9-phenylxanthen-9-yl (Pixyl) and 9-(p-methoxyphenyl)xanthen-9-yl (Mox). The protecting group can be removed from oligonucleotides of the conjugated oligomeric compound of the invention by techniques well known in the art to form the free hydroxy. For example, dimethoxytrityl protecting groups can be removed by protic acids such as formic acid, dichloroacetic acid, trichloroacetic acid, p-toluene sulfonic acid or with Lewis acids such as for example zinc bromide.

Following assembly of the desired oligomeric compound, the next step will normally be deprotection of nucleic bases and internucleosidic phosphates of the oligomeric compound and cleavage of the synthesized oligomeric compound from the support medium. These processes can take place substantially simultaneously, thereby providing the free oligomeric compound in the desired form.

Preferably, the process of the invention further comprises the step of treating said oligomeric compound with a reagent effective to cleave said oligomeric compound from said support medium. Preferred cleaving reagents include gaseous ammonia, alkylamines including methylamine, ethylamine, or propylamine, solutions of ammonia, alkylamines, including methylamine, ethylamine, propylamine, t-butylamine, piperidine, pyrrolidine, piperazine in water or organic solvents, solutions of alkalis, lithium hydroxide, sodium hydroxide, potassium hydroxide in water or organic solvents including methanol, ethanol, propanol, or isopropanol, solutions of lithium carbonate, sodium carbonate, or potassium carbonate in water or organic solvents including methyl alcohol, or ethyl alcohol.

Preferably, the process further comprises the step of treating said oligomeric compound with a reagent effective to remove protecting groups from said oligomeric compound. Preferred deprotecting reagents include gaseous ammonia, alkylamines including methylamine, ethylamine, or propylamine, solutions of ammonia, alkylamines including methylamine, ethylamine, propylamine, t-butylamine, piperidine, pyrrolidine, piperazine in water or organic solvents, solutions of alkalis, lithium hydroxide, sodium hydroxide, potassium hydroxide in water or organic solvents including methanol, ethanol, propanol, or isopropanol, solutions of lithium carbonate, sodium carbonate, or potassium carbonate in water or organic solvents including methyl alcohol, or ethyl alcohol.

The support media useful with the compound and in the processes of the invention are used for attachment of a first nucleoside or other monomeric subunit that is then iteratively elongated to give a final oligomeric compound. Support media may be selected to be insoluble or have variable solubility in different solvents to allow the growing support bound polymer to be either in or out of solution as desired. Traditional support media such as solid supports are generally insoluble and are routinely placed in a reaction vessel while reagents and solvents react and/or was the growing chain until cleavage the final polymeric compound. More recent approaches have introduced soluble supports including soluble polymer supports to allow precipitating and dissolving and iteratively synthesized product at desired points in the synthesis (Graver et al., Chem. Rev., 1997, 97, 489-510).

The current method of choice for the preparation of oligomeric compound utilizes support media. Support media are used for attachment of first nucleoside or other monomeric subunit that is then iteratively elongated to give a final oligomeric compound or other polymer such as polypeptide. Support media can be selected to be insoluble or have variable solubility in different solvents to allow the growing support bound polymer to be either in or out of solution as desired. Traditional support media such as solid supports are, for the most part, insoluble and are routinely placed in a reaction vessel while reagents and solvents react and or wash the growing chain until cleavage the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-5 10).

The term “support media” is intended to include all forms of support known to skilled artisans for the synthesis of oligomeric compounds and related compound such as peptides. Some representative support media that are amenable to the methods of the present invention include but are not limited to the following: controlled pore glass (CPG); oxalyl-controlled pore glass (see, e.g.g, Alul, et al., Nucleic Acids Research 1991, 19, 1527); silica-containing particles, such as porous glass beads and silica gel such as that formed by the reaction of trichloro-[3-(4-chloromethyl)phenyl]propylsilane and porous glass beads (see Parr and Grohmann, Angew. Chem. Internal. Ed. 1927, 11, 314, sold under the trademark “PORASILE” by Waters Associates, Framingham, Mass., USA); the mono ester of 1,5-dihydroxymethylbenzene and silica (see Bayer and Jung, Tetrahedron Lett., 1970, 4503, sold under the trademark “BIOPAK” by Waters Associates); TENTAGEL (see, e.g., Wright, et al., Tetradhedron Letters 1993, 34, 3373); cross-linked styrene/divinylbenzene copolymer beaded matrix or POROS, a copolymer of polystyrene/divinylbenzene (available from Perceptive Biosystems); soluble support media, polyetherhylene glycol (see Bonora et al., Oragnic Process Research & Development, 2000, 4, 225-231).

Further support media amenable to the present invention include without limitation PEPS support a polyethylene (PE) film with pendant long-chain polystyrene (PS) grafts (molecular weight on the order of 10⁶, (See Berg, et al., J. Amer. Chem. Soc., 1989, 111, 8024 and International Patent Application WO 90/02749),).

Further support media amenable to the present invention include without limitation particles based upon copolymers of dimethylacrylamide cross-linked with N,N′-bisacryloylehtylenediamine, including a known amount of N-tertbutoxycarbonyl-beta-alanyl-N′-acryloylhexamethylenediamine. Several spacer molecules are typically added via the beta alanyl groups, followed thereafter by the amino acids residue subunits. Also, the beta alanyl-containing monomer can be replaced with an acryloyl sarcosine monomer during polymerization to form resin beads. The polymerization if followed by reaction of the beads with ethylenediamine to from resin particles that contain primary amines as the covalently linked functionality. The polyacrylamide-based supports are relatively more hydrophilic than are the polystyrene-based supports and are usually sued with polar aprotic solvents including dimethylformamide, dimethylacetamide, N-methylpyrrolidone and the like (see Atherton, et al., J. Amer. Chem. Soc., 1975, 97, 6584, Bioorg. Chem. 1979, 8, 351, and J. Chem. Soc. Perkin Trans. I 1981, 538).

Further support media amenable to the present invention include without limitation a composite of a resin and another material that is also substantially inert to the organic synthesis reaction conditions employed. One exemplary composite (see Scott, et al., J. Chrom. Sci., 1971, 9, 577) utilizes glass particles coated with a hydrophobic, cross-linked styrene polymer containing reactive chloromethyl groups, and is supplied by Northgate Laboratories, Inc., of Hamden, Conn., USA. Another exemplary composite contains a core of fluorinated ethylene polymer onto which has been grafted polystyrene (see Kent and Merrifield, Israel J. Chem. 1978, 17, 243 and van Rietschoten in Peptides 1974, Y. Wolman, Ed., Wiley and Sons, New York, 1975, pp. 113-116).

Support bound oligonucleotide synthesis relies on sequential addition of nucleotides to one end of a growing chain. Typically, first nucleoside (having protecting groups on any exocyclic amine functionalities present) is attached to an appropriate glass bead support and activated phosphite compound (typically nucleotide phosphoramidites, also bearing appropriate protecting groups) are added stepwise to elongate the growing oligonucleotides. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069. In some especially preferred embodiments, the nucleoside components of the oligomeric compounds are connected to each other by optionally protected phosphorothioate internucleoside linkages. Representative protecting groups for phosphorus containing internucleoside linkages such as phosphite, phosphodiester and phosphorothioate linkages include β-cyanoethyl, diphenylsilylethyl, δ-cyanobutenyl, cyano p-xylyl (CPX), N-methyl-N-trifluoroacetyl ethyl (META), acetoxy phenoxy ethyl (APE) and butene-4-yl groups. See for example U.S. Pat. Nos. 4,725,677 and Re. 34,609 (β-cyanoethyl); Beaucage, S. L. and Iyer, R. P. Tetrahedron, 1993, 49, 1925-1963; Beaucage, S. L. and Iyer, R. P., Tetrahedron, 1993, 49, 10441-10488; Beaucage, S. L. and Iyer, R. P., Tetrahedron, 1992, 48, 2223-2311. The use of 2-benzamidoethyl protecting groups is disclosed in Guzaev, A. P.; Manoharan, M. J. Amer. Chem. Soc. 2001, 123, 783-793; U.S. Pat. Nos. 6,121,437 and 6,610,837. Other representative phosphorus protecting groups include —CH₂CH═CHCH₂CN, p-C₆H₄CH₂CN, —(CH₂)₂—N(H)COCF₃, —CH₂CH₂Si(C₆H₅)₂(CH₃)₂, —CH₂CH₂ N(CH₃)COCF₃ and other known in the art.

As used herein, the use in the lists in methods or compositions of numbers and letters does not imply any specific sequence or priority, unless explicitly stated.

The following examples are illustrative but are not meant to be limiting of the present invention.

### EXAMPLES

### Example 1

### Universal Solid Support 5

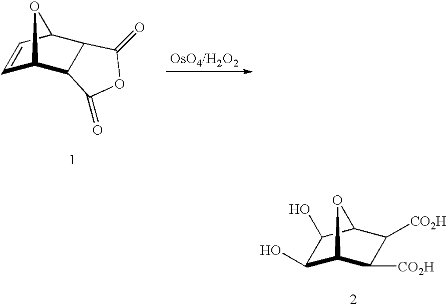

### (1α,2α,3α,4α,5α,6α)-5,6-dihydroxy-7-oxabicyclo[2.2.1]heptane-2,3-dicarboxylic acid (2)

A solution of commercial (3aR,4S,7R,7aS)-rel-3a,4,7,7a-tetrahydro-4,7-epoxyisobenzofuran-1,3-dione, 1, (8.31 g, 50.0 mmol) in hydrogen peroxide (35% aqueous, 7.29 g, 75.0 mmol), acetonitrile (80.0 mL), and water (10.0 mL) was treated with osmium tetroxide (13 mg, 0.05 mmol) in t-butanol (0.51 mL) for 1 h at 60-65° C. The reaction mixture was cooled to room temperature, treated with ether (90 mL), and kept at 4° C. for 1 h. The precipitate was filtered off, washed with ether and dried to give pure 2 (8.54 g, 84.5%). The compound may be optionally re-crystallized from ethanol.

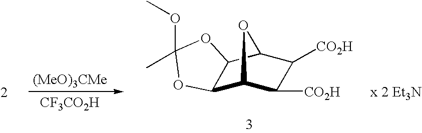

### Bis-triethylammonium (1α,2α,3α,4α,5α,6α)-5,6-O-(1-methoxyethylydene)-5,6-dioxy-7-oxabicyclo[2.2.1]heptane-2,3-dicarboxylate (3)

A solution of compound 2 (2.18 g, 10.0 mmol), trimethyl orthoacetate (1.50 g, 12.5 mmol), and trifluoroacetic acid (0.023 g, 0.2 mmol) in DMF (50 mL) was stirred overnight and neutralized with triethylamine (5 mL). The solvent was evaporated, and the residue was dissolved in a mixture of triethylamine and aqueous ethanol (5:15:80; 100 mL) and filtered through a short pad of silica gel. The silica gel was washed with 85% aqueous ethanol (100 mL), and the combined solution was evaporated. The residue was co-evaporated with ethanol and treated with ethyl acetate (200 mL). The viscous precipitate (4.70 g, 98.6%) was dried in vacuo and used in the next synthetic step without any further purification.

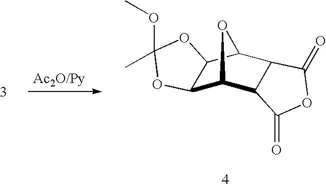

### (1α,2α,3α,4α,5α,6α)-5,6-O-(1-methoxyethylydene)-5,6-dioxy-7-oxabicyclo[2.2.1]heptane-2,3-dicarbanhydride (4)

Compound 3 (477 mg, 1.0 mmol) was treated with acetic anhydride (300 mg) and pyridine (5 mL) for 30 min at room temperature. The mixture was evaporated and co-evaporated with pyridine (5×15 mL) to give the title compound (255 mg, 99.5%) as a colorless foam, which was used in the next step without any further purification.

Universal solid support 5.

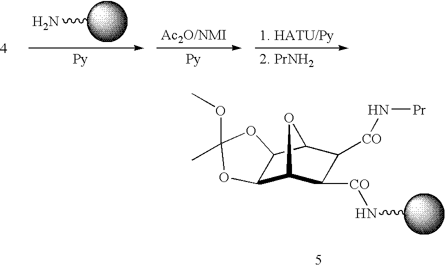

Aminoalkyl controlled pore glass (2667 mg, 0.5 mmol) was gently shaken with compound 4 (256 mg, 1.0 mmol) in pyridine (10 mL) overnight. The suspension was filtered, and the solid support was washed with pyridine (3×20 mL). The collected solution was evaporated, the residue was, upon treatment with acetic anhydride as described above, stored for loading another portion of aminoalkyl CPG. The solid support was additionally washed with ethyl acetate, dried, and capped by treating with a mixture of Ac₂O/pyridine/N-methylimidazole/THF (10:10:10:70) for 3 h at room temperature. Finally, the solid support was washed with MeCN. The resulting solid support was treated with 0.2 M HATU in MeCN-pyridine (4:1, 6 mL) for 5 min. The liquid phase was removed, and the solid support was treated with 0.5 M propylamine in MeCN (5 mL) for 15 min. The solid support 5 was washed with MeCN and ethyl acetate and dried. The aliquot of the solid support 5 (10 mg) was detritylated and treated with a commercial 2-cyanoethyl T phosphoramidite in the presence of 1H-tetrazole using the standard protocol of oligonucleotide synthesis. The resulting solid support was dried and the loading of 60 μmol g⁻¹ was determined by the standard dimethoxytrityl assay.

### Example 2

### Universal Solid Support 6

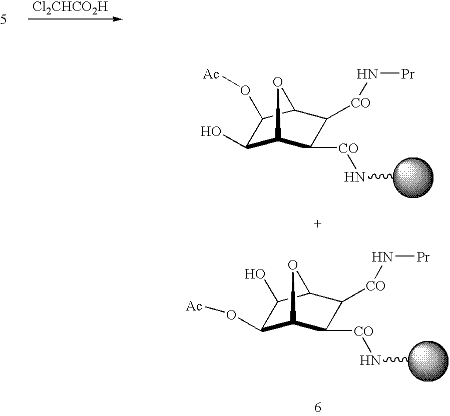

The solid support 5 (2.0 g) was washed with 3% dichloroacetic acid (20 mL) for 5 min followed by washing with pyridine (3×10 mL). Finally, the solid support 6 was washed with MeCN and dried.

### Example 3

### Universal Solid Support 9

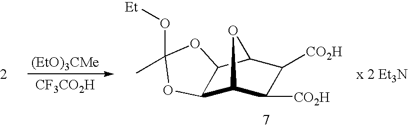

### Bis-triethylammonium (1α,2α,3α,4α,5α,6α)-5,6-O-(1-ethoxyethylydene)-5,6-dioxy-7-oxabicyclo[2.2.1]heptane-2,3-dicarboxylate (7)

A solution of compound 2 (2.18 g, 10.0 mmol), triethyl orthoacetate (2.03 g, 12.5 mmol), and trifluoroacetic acid (0.023 g, 0.2 mmol) in DMF (50 mL) was stirred overnight and neutralized with tethylamine (5 mL). The solvent was evaporated, and the residue was treated with ethyl acetate (200 mL). The viscous precipitate was dried in vacuo and used in the next synthetic step without any further purification.

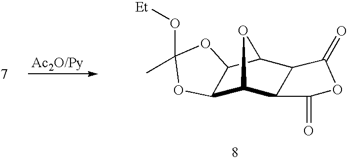

### (1α,2α,3α,4α,5α,6α)-5,6-O-(1-ethoxyethylydene)-5,6-dioxy-7-oxabicyclo[2.2.1]heptane-2,3-dicarbanhydride (8)

Compound 7 (490 mg, 1.0 mmol) was treated with acetic anhydride (300 mg) and pyridine (5 mL) for 30 min at room temperature. The mixture was evaporated and co-evaporated with pyridine (5×15 mL) to give the title compound (264 mg, 97.8%) as a colorless foam, which was used in the next step without any further purification.

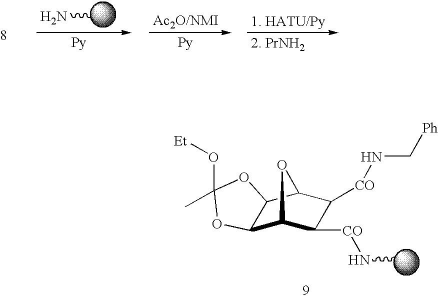

### Universal solid support 9

Aminoalkyl controlled pore glass (2667 mg, 0.5 mmol) was gently shaken with compound 8 (270 mg, 1.0 mmol) in pyridine (10 mL) overnight. The suspension was filtered, and the solid support was washed with pyridine (3×20 mL). The collected solution was evaporated, the residue was, upon treatment with acetic anhydride as described above, stored for loading another portion of aminoalkyl CPG. The solid support was additionally washed with ethyl acetate, dried, and capped by treating with a mixture of Ac₂O/pyridine/N-methylimidazole/THF (10:10:10:70) for 3 h at room temperature. Finally, the solid support was washed with MeCN. The resulting solid support was treated with 0.2 M HATU in MeCN-pyridine (4:1, 6 mL) for 5 min. The liquid phase was removed, and the solid support was treated with 0.5 M benzylamine in MeCN (5 mL) for 15 min. The solid support 9 was washed with MeCN and ethyl acetate and dried. The aliquot of the solid support 9 (10 mg) was detritylated and treated with a commercial 2-cyanoethyl T phosphoramidite in the presence of 1H-tetrazole using the standard protocol of oligonucleotide synthesis. The resulting solid support was dried and the loading of 56 μmol g⁻¹ was determined by the standard dimethoxytrityl assay.

### Example 4

### Universal Solid Support 12

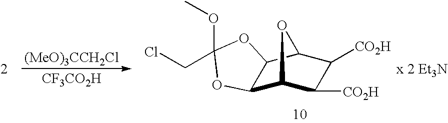

### Bis-triethylammonium (1α,2α,3α,4α,5α,6α)-5,6-O-(1-methoxy-2-chloroethylydene)-5,6-dioxy-7-oxabicyclo[2.2.1]heptane-2,3-dicarboxylate (10)

A solution of compound 2 (2.18 g, 10.0 mmol), commercial trimethyl orthochloroacetate (Aldrich; 1.78 g, 11.5 mmol), and trifluoroacetic acid (0.023 g, 0.2 mmol) in DMF (50 mL) was stirred overnight and neutralized with triethylamine (5 mL). The solvent was evaporated, and the residue was dissolved in a mixture of triethylamine and aqueous ethanol (5:15:80; 100 mL) and filtered through a short pad of silica gel. The silica gel was washed with 85% aqueous ethanol (100 mL), and the combined solution was evaporated. The residue was co-evaporated with ethanol and treated with ethyl acetate (200 mL). The viscous precipitate (4.81 g, 94.1%) was dried in vacuo and used in the next synthetic step without any further purification.

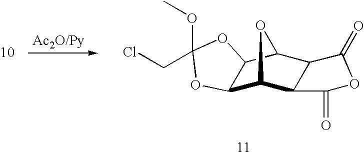

### (1α,2α,3α,4α,5α,6α)-5,6-O-(1-methoxy-2-chloroethylydene)-5,6-dioxy-7-oxabicyclo[2.2.1]heptane-2,3-dicarbanhydride (11)

Compound 10 (511 mg, 1.0 mmol) was treated with acetic anhydride (300 mg) and pyridine (5 mL) for 30 min at room temperature. The mixture was evaporated and co-evaporated with pyridine (5×15 mL) to give the title compound as a colorless foam (288 mg, 99%), which was used in the next step without any further purification.

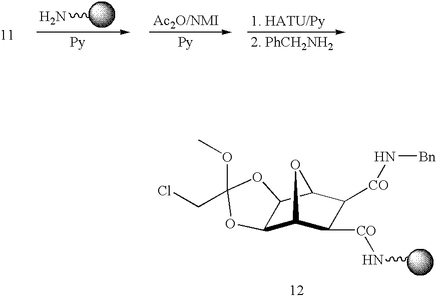

### Universal Solid Support 12

Aminoalkyl controlled pore glass (2667 mg, 0.5 mmol) was gently shaken with compound 11 (256 mg, 1.0 mmol) in pyridine (10 mL) overnight. The suspension was filtered, and the solid support was washed with pyridine (3×20 mL). The collected solution was evaporated, the residue was, upon treatment with acetic anhydride as described above, stored for loading another portion of aminoalkyl CPG. The solid support was additionally washed with ethyl acetate, dried, and capped by treating with a mixture of Ac₂O/pyridine/N-methylimidazole/THF (10:10:10:70) for 3 h at room temperature. Finally, the solid support was washed with MeCN. The resulting solid support was treated with 0.2 M HATU in MeCN-pyridine (4:1, 6 mL) for 5 min. The liquid phase was removed, and the solid support was treated with 0.5 M benzylamine in MeCN (5 mL) for 15 min. The aliquot of the solid support 12 (10 mg) was detritylated and treated with a commercial 2-cyanoethyl T phosphoramidite in the presence of 1H-tetrazole using the standard protocol of oligonucleotide synthesis. The resulting solid support was dried and the loading of 52 μmol g⁻¹ was determined by the standard dimethoxytrityl assay.

### Example 5

### Universal Solid Support 13

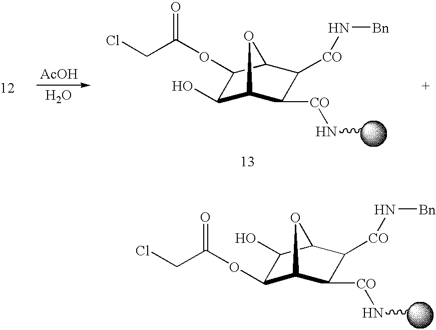

### Universal Solid Support 13

The solid support 12 (500 mg) was treated with 80% aqueous acetic acid (4 mL) for 10 min. The suspension was filtered, and the solid support was washed with pyridine (3×10 mL). Finally, the solid support 12 was washed with MeCN and ethyl acetate and dried.

### Example 6

### Universal Solid Support 19

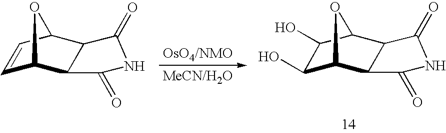

### Exo-5,6-dihydroxy-7-oxabicyclo[2.2.1]heptane-2,3-dicarboximide (14) A solution of exo-7-oxabicyclo[2.2.1]hept-4-ene-2,3-dicarboximide prepared as described in [Kwart, H.; Burchuk, I. J. Amer. Chem. Soc. 1952, 74, 3094-3097] (5.14 g, 32.20 mmol) and N-methylmorpholine-N-oxide (4.72 g, 40.25 mmol) in acetonitrile (40.0 mL) and water (15.0 mL) was treated with osmium tetroxide (20 mg) in t-butanol (1.0 mL) for 3 h at 40-45° C. The reaction mixture was evaporated and co-evaporated with acetonitrile (4×50 mL). The solid residue was re-crystallized from aqueous ethanol to give compound 14 (3.50 g, 56.11%) as a white solid.

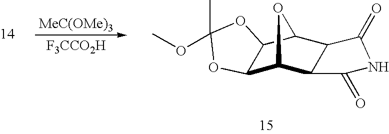

### (3aR,4R,5S,6R,7R,7aR)-rel-Hexahydro-2-methyl-2-methoxy-4,7-epoxy-1,3-benzodioxole-5,6-dicarboximide (15)

A mixture of compound 14 (18.50 g, 92.90 mmol), trimethyl orthoacetate (16.74 g, 139.3 mmol), trifluoroacetic acid (211 mg), and DMF (90 mL) was shaken for 24 h. Ethyldiisopropylamine (8.0 mL) was added, and the solvent was evaporated. The residue was dissolved in ethyl acetate (200 mL) and washed with 5% aqueous NaHCO₃ (3×20 mL) followed by brine (20 mL). The organic phase was dried over Na₂SO₄ and evaporated. The residue was re-crystallized from a mixture of toluene and ethylacetate to give compound 15 (24.0 g, 98.2%).

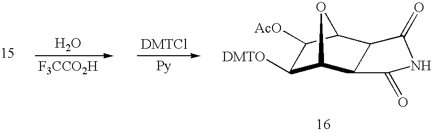

### Exo-5-acetoxy-6-(4,4′-dimethoxytrityloxy)-7-oxabicyclo[2.2.1]heptane-2,3-dicarboximide (16)

A solution of compound 15 (14.20 g, 35.50 mmol) and trifluoroacetic acid (127 mg) in acetonitrile (60 mL) was treated with water (10 mL) for 15 min. The solvent was evaporated, the residue was co-evaporated with acetonitrile (3×50 mL).

The solution of the product (8.56 g, 35.5 mmol) in pyridine (60 mL) was treated with dimethoxytrityl chloride (14.43 g, 42.6 mmol) overnight and the solvent was evaporated. The residue was dissolved in ethyl acetate (400 mL) and washed with 5% aqueous NaHCO₃ (3×50 mL) followed by brine (100 mL), dried over Na₂SO₄, and evaporated. The residue was purified on a silica gel column eluting with a gradient of MeOH in CH₂Cl₂ plus pyridine (1%) to give compound 16 (19.30 g,75.88%).

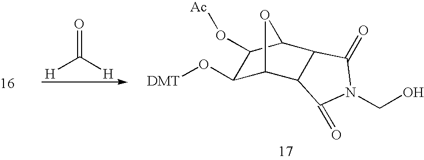

### Exo-N-hydroxymethyl-5-acetoxy-6-(4,4′-dimethoxytrityloxy)-7-oxabicyclo[2.2.1]heptane-2,3-dicarboximide (17)

A solution of compound 16 (2.72 g, 5.00 mmol) and 37% aqueous formaldehyde (0.61 g, 7.50 mmol) in pyridine (3.0 mL) and acetonitrile (30 mL) was stirred for 3 h. The mixture was evaporated and dissolved in ethyl acetate (100 mL). The solution was washed with water (3×50 mL) followed by brine (100 mL), dried over Na₂SO₄, and evaporated to give compound 17 (2.86 g, 99.7%) in more than 97% purity.

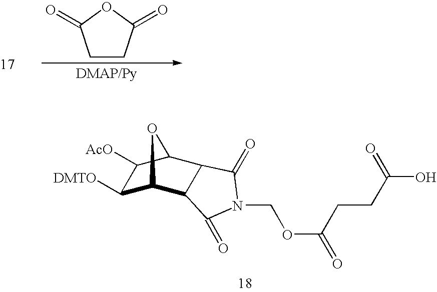

### Triethylammonium (3aR,4R,5S,6R,7R,7aR)-rel-N-((3-hemisuccinyloxy)methyl)-5-acetoxy-6-(4,4′-dimethoxytrityloxy)-7-oxabicyclo[2.2.1]heptane-2,3-dicarboximide (18)

A solution of compound 17 (544 g, 1.0 mmol), succinic anhydride (300 mg, 3.0 mmol), and 4-dimethylaminopyridine (36 mg, 0.3 mmol) in pyridine (0.3 mL) and acetonitrile (3.0 mL) was kept overnight. The solvent was evaporated, the residue was treated with ethyl acetate (50 mL) and 1 M aqueous triethylammonium acetate (10 mL). The organic solution was washed with 1 M aqueous triethylammonium acetate (4×10 mL), water (3×10 mL) dried over Na₂SO₄, and evaporated. The residue was dissolved in ethyl acetate (5 mL) and precipitated into hexane (100 mL). The precipitate was collected, washed with hexane, and dried in vacuo to give 18 (723 mg, 93.3%) as a white solid.

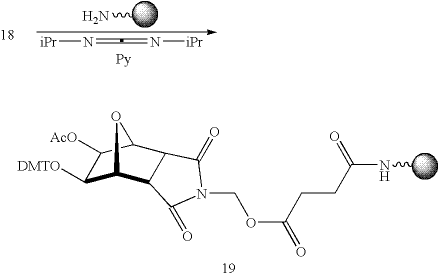

### Universal Solid Support 19

A mixture of compound 18 (723 mg, 0.93 mmol), long chain aminoalkyl controlled pore glass (2.00 g, 100 μmol g⁻¹, 0.20 mmol), N,N′-diisopropylcarbodiimide (252 mg, 2.0 mmol), and DMAP (12 mg, 0.1 mmol) in pyridine (10 mL) was shaken overnight. The solid phase was filtered, washed with pyridine (3×10 mL), acetonitrile (3×10 mL), and capped with a mixture of Ac₂O/pyridine/N-methylimidazole/THF (10:10:10:70; 20 mL) for 3 h. Finally, the solid phase was washed with MeCN and ethyl acetate and dried to give the solid support 19 loaded at 20.0 μmol g⁻¹.

### Example 7

### Universal Solid Support 22

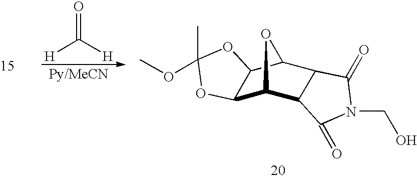

### (3aR,4R,5S,6R,7R,7aR)-rel-N-(Hydroxymethyl)-hexahydro-2-methyl-2-methoxy-4,7-epoxy-1,3-benzodioxole-5,6-dicarboximide (20)

A solution of compound 15 (510 mg, 2.00 mmol) and 37% aqueous formaldehyde (178 mg, 2.20 mmol) in pyridine (0.3 mL) and acetonitrile (4.7 mL) was stirred for 24 h. The mixture was evaporated and dissolved in ethyl acetate (50 mL). The solution was washed with water (3×25 mL) followed by brine (100 mL), dried over Na₂SO₄, and evaporated to give compound 20 (509 mg, 89.3%) in more than 97% purity.

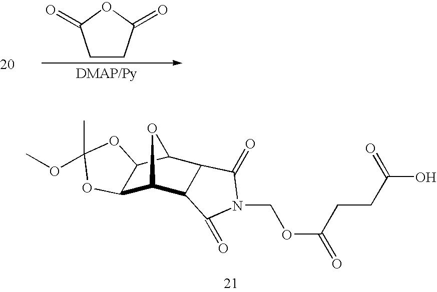

### (3aR,4R,5S,6R,7R,7aR)-rel-N-((Succinyloxy)methyl)-hexahydro-2-methyl-2-methoxy-4,7-epoxy-1,3-benzodioxole-5,6-dicarboximide (21)

A solution of compound 20 (480 mg, 1.68 mmol), succinic anhydride (336 mg, 3.36 mmol), and 4-dimethylaminopyridine (21 mg, 0.17 mmol) in pyridine (0.3 mL) and acetonitrile (3.0 mL) was kept overnight. The solvent was evaporated, the residue was treated with ethyl acetate (50 mL) and 1 M aqueous triethylammonium acetate (10 mL). The organic solution was washed with 1 M aqueous triethylammonium acetate (4×10 mL), water (3×10 mL) dried over Na₂SO₄, and evaporated. The residue was dissolved in ethyl acetate (5 mL) and precipitated into hexane (100 mL). The precipitate was collected, washed with hexane, and dried in vacuo to give 21 (504 mg, 77.9%) as a white solid.

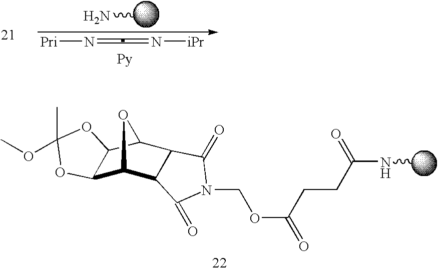

### Universal Solid Support 22

A mixture of compound 21 (475 mg, 1.23 mmol), long chain aminoalkyl controlled pore glass (5.00 g, 100 μmol g⁻¹, 0.5 mmol), N,N′-diisopropylcarbodiimide (233 mg, 1.85 mmol), and DMAP (12 mg, 0.1 mmol) in pyridine (25 mL) was shaken overnight. The solid phase was filtered, washed with pyridine (3×20 mL), acetonitrile (3×20 mL), and capped with a mixture of Ac₂O/pyridine/N-methylimidazole/THF (10:10:10:70; 50 mL) for 3 h. Finally, the solid phase was washed with MeCN and ethyl acetate and dried to give the solid support 22. The aliquot of the solid support 22 (10 mg) was detritylated and treated with a commercial 2-cyanoethyl T phosphoramidite in the presence of 1H-tetrazole using the standard protocol of oligonucleotide synthesis. The resulting solid support was dried and the loading of 31.4 μmol g⁻¹ was determined by the standard dimethoxytrityl assay.

### Example 8

### Universal Solid Support 23

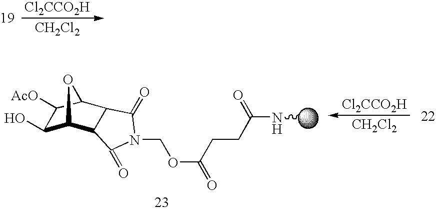

From universal solid support 19. The solid support 19 (500 mg) was washed on a filter with dichloroacetic acid (3% in CH2Cl2) for about 3 min until no orange-colored product was eluted from the solid support. The solid phase was filtered, washed with pyridine (3×20 mL), acetonitrile (3×20 mL) and dried to give the solid support 23.

From universal solid support 22. The solid support 17 (500 mg) was washed on a filter with dichloroacetic acid (3% in CH2Cl2) or a mixture of acetic acid, water, and MeCN (5:15:80) for about 3 min. The solid phase was filtered, washed with pyridine (3×20 mL), acetonitrile (3×20 mL) and dried to give the solid support 23.

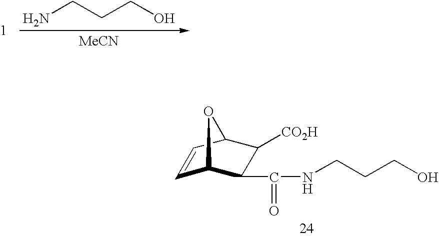

### Example 9

### Universal Solid Support 29

### Exo-N-(3-hydroxypropyl)-7-oxabicyclo[2.2.1]hept-5-ene-3-carboxamide-2-carboxylic acid (24)

A solution of compound 1 (12.46 g, 75.0 mmol) in MeCN (400 mL) and pyridine (25 mL) was treated with 3-aminopropanol (5.75 g, 76.5 mmol) in MeCN (50 mL) in an ice-water bath for 45 min and at room temperature for 2 h. The precipitate was filtered off and washed on the filter with MeCN (3×50 mL). The precipitate was dried in vacuo to give ca. 98% pure compound 24 (16.98 g, 93.82%), which was used in the next synthetic step without any further purification.

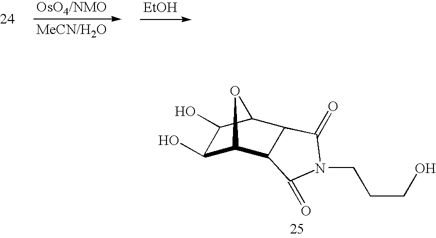

### Exo-N-(3-hydroxypropyl)-5,6-dihydroxy-7-oxabicyclo[2.2.1]heptane-2,3-dicarboximide (25)

A solution of compound 24 (16.53 g, 68.5 mmol) and N-methylmorpholine-N-oxide (8.43 g, 71.9 mmol) in acetonitrile (80.0 mL) and water (20.0 mL) was treated with osmium tetroxide (17 mg, 0.069 mmol) in t-butanol (0.9 mL) for 1 h at 60-65° C. The reaction mixture was evaporated and co-evaporated with acetonitrile (2×50 mL). The residue was suspended in ethanol (200 mL) and boiled with a reflux condenser for 3 h. The mixture was diluted with ethanol (140 mL) and water (60 mL) and filtered through a short pad of silica gel. Silica gel was washed with 85% aqueous ethanol (200 mL), and the combined solution was evaporated. The solid residue was re-crystallized from 95% ethanol to give compound 25 (12.61 g, 71.6%) as an off-white solid.

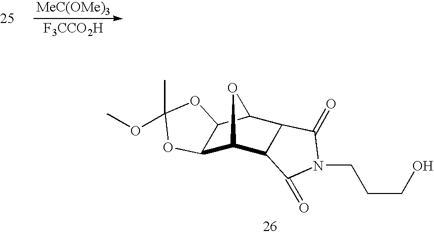

### (3aR,4R,5S,6R,7R,7aR)-rel-N-(3-hydroxypropyl)-hexahydro-2-methyl-2-methoxy-4,7-epoxy-1,3-benzodioxole-5,6-dicarboximide (26)

A mixture of compound 25 (3.58 g, 13.9 mmol), trimethyl orthoacetate (2.17 g, 18.1 mmol), trifluoroacetic acid (130 mg), and acetonitrile (60 mL) was shaken overnight. Triethylamine (2.0 mL) was added, and the solvent was evaporated. The residue was dissolved in ethyl acetate (200 mL) and washed with 5% aqueous NaHCO₃ (3×20 mL) followed by brine (20 mL). The organic phase was dried over Na₂SO₄ and evaporated. The residue was purified on a silica gel column eluting with a gradient of MeOH in CH₂Cl₂ to give compound 26 (3.42 g, 91.4%).

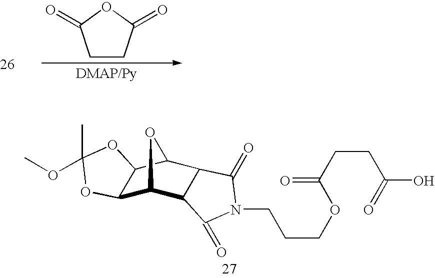

### (3aR,4R,5S,6R,7R,7aR)-rel-N-((3-hemisuccinyloxy)propyl)-hexahydro-2-methyl-2-methoxy-4,7-epoxy-1,3-benzodioxole-5,6-dicarboximide (27)

A solution of compound 26 (5.33 g, 17.0 mmol), succinic anhydride (2.55 g, 25.5 mmol), and 4-dimethylaminopyridine (207 mg, 1.7 mmol) in pyridine (25 mL) was kept overnight. The solvent was evaporated, the residue was treated with ethyl acetate (200 mL) and 1 M aqueous triethylammonium acetate (20 mL). The organic solution was washed with 1 M aqueous triethylammonium acetate (4×20 mL), water (3×20 mL) dried over Na₂SO₄, and evaporated. The residue was dissolved in ethyl acetate (50 mL) and precipitated into hexane (500 mL). The precipitate was collected, washed with hexane, and dried in vacuo to give 27 (6.02 g, 85.7%) as an off-white solid.

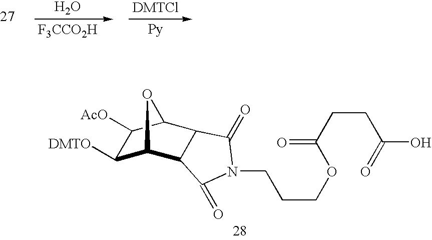

### Exo-N-(3-(hemisuccinyloxy)propyl)-5-acetoxy-6-(4,4′-dimethoxytrityloxy)-7-oxabicyclo[2.2.1]heptane-2,3-dicarboximide (28)

A solution of compound 27 (6.82 g, 16.5 mmol) and trifluoroacetic acid (19 mg) in acetonitrile (100 mL) was treated with water (2 mL) for 15 min. The solvent was evaporated, the residue was co-evaporated with pyridine (3×50 mL), and dissolved in pyridine (50 mL).

The solution was treated with dimethoxytrityl chloride (6.15 g, 18.2 mmol) overnight and the solvent was evaporated. The residue was dissolved in ethyl acetate (200 mL) and washed with 1 M aqueous triethylammonium acetate (4×20 mL), water (3×20 mL), dried over Na₂SO₄, and evaporated. The residue was dissolved in ethyl acetate (50 mL) and precipitated into hexane (500 mL). The residue was purified on a silica gel column eluting with a gradient of MeOH in CH₂Cl₂ plus triethylamine (5%) to give compound 28 (10.92 g, 94.4%).

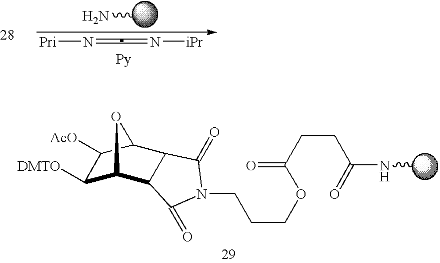

### Universal Solid Support 29

A mixture of compound 28 (2.76 g, 3.94 mmol), long chain aminoalkyl controlled pore glass (26.34 g, 115 μmol g⁻¹, 3.03 mmol), N,N′-diisopropylcarbodiimide (597 g, 4.73 mmol), and DMAP (375 mg, 3.07 mmol) in pyridine (130 mL) was shaken overnight. The solid phase was filtered, washed with pyridine (3×100 mL), acetonitrile (3×100 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 29 loaded at 41.1 μmol g⁻¹.

### Example 10

### Universal Solid Support 30

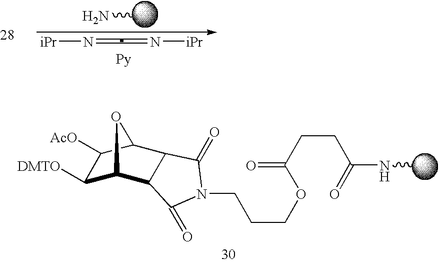

A mixture of compound 28 (526 mg, 0.75 mmol), amino polystyrene (5.00 g, 75 μmol g⁻¹, 0.375 mmol), N,N′-diisopropylcarbodiimide (142 mg, 1.13 mmol), and DMAP (92 mg, 0.75 mmol) in pyridine (30 mL) was shaken overnight. The solid phase was filtered, washed with pyridine (3×50 mL), acetonitrile (3×50 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 30. The aliquot of the solid support 13 (10 mg) was detritylated and treated with a commercial 2-cyanoethyl T phosphoramidite in the presence of 1H-tetrazole using the standard protocol of oligonucleotide synthesis. The resulting solid support was dried and the loading of 15.1 μmol g⁻¹ was determined by the standard dimethoxytrityl assay.

### Example 11

### Universal Solid Support 31

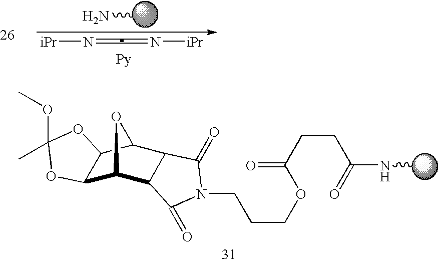

A mixture of compound 26 (3.63 g, 8.81 mmol), long chain aminoalkyl controlled pore glass (51.0 g, 115 μmol g⁻¹, 5.87 mmol), N,N′-diisopropylcarbodiimide (1.67 g, 13.22 mmol), and DMAP (1.07 g, 8.81 mmol) in pyridine (250 mL) was shaken overnight. The solid phase was filtered, washed with pyridine (3×300 mL), acetonitrile (3×300 mL), and capped with a mixture of Ac₂O/pyridine/N-methylimidazole/THF (10:10:10:70; 300 mL) for 3 h. Finally, the solid phase was washed with MeCN and ethyl acetate and dried to give the solid support 31. The aliquot of the solid support 31 (10 mg) was detritylated and treated with a commercial 2-cyanoethyl T phosphoramidite in the presence of 1H-tetrazole using the standard protocol of oligonucleotide synthesis. The resulting solid support was dried and the loading of 44.4 μmol g⁻¹ was determined by the standard dimethoxytrityl assay.

### Example 12

### Universal Solid Support 32

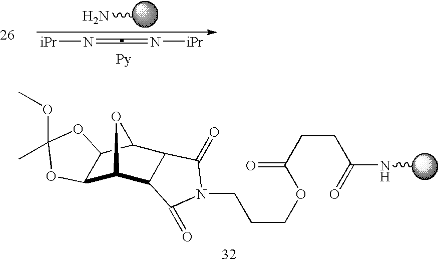

A mixture of compound 26 (309 mg, 0.75 mmol), amino polystyrene (5.00 g, 75 μmol g⁻¹, 0.375 mmol), N,N′-diisopropylcarbodiimide (142 mg, 1.13 mmol), and DMAP (92 mg, 0.75 mmol) in pyridine (30 mL) was shaken overnight. The solid phase was filtered, washed with pyridine (3×50 mL), acetonitrile (3×50 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 32. The aliquot of the solid support 32 (10 mg) was detritylated and treated with a commercial 2-cyanoethyl T phosphoramidite in the presence of 1H-tetrazole using the standard protocol of oligonucleotide synthesis. The resulting solid support was dried and the loading of 16.9 μmol g⁻¹ was determined by the standard dimethoxytrityl assay.

### Example 13

### Universal Solid Support 33

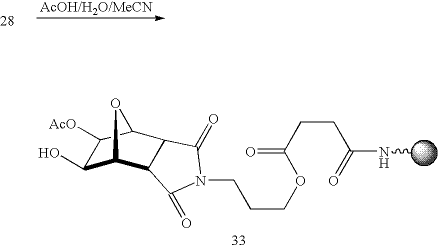

A compound 32 (100 mg, 16.9 μmol g⁻¹) was washed with 3% dichloroacetic acid in CH₂Cl₂ (5 mL) for 1 min. The solid phase was washed with acetonitrile (3×5 mL), and dried to give the solid support 33 (100 mg) loaded at 16.9 μmol g⁻¹.

### Example 14

### Universal Phosphoramidite 34 and Universal Solid Support 34a

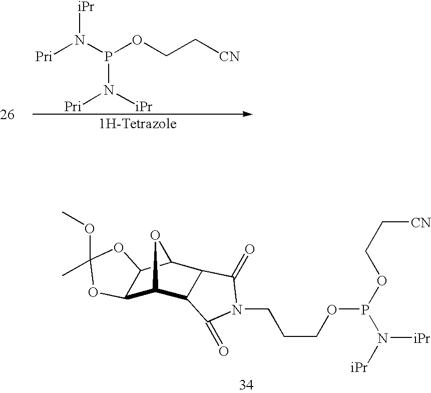

### 2-Cyanoethyl 3-(exo-5-acetoxy-6-(4,4′-dimethoxytrityloxy)-7-oxabicyclo[2.2.1]heptane-2,3-dicarboximido)propyl N,N-diisopropylphosphoramidite (34)

A solution of compound 26 (3.13 g, 10.0 mmol) and 2-cyanoethyl N,N,N′,N′-tetraisopropylphosphorodiamidite (3.31 g, 10.5 mmol) in MeCN (20 mL) was treated with 1H-tetrazole (0.45 M in MeCN, 11.1 mL, 5.0 mmol) for 6 h. The reaction mixture was quenched with 5% aqueous NaHCO₃ (50 mL) and diluted with ethyl acetate (300 mL). The solution was washed with 5% aqueous NaHCO₃ (50 mL), brine (3×50 mL), dried over Na₂SO₄ and evaporated. The residue was separated on a silica gel column eluting with a gradient of ethyl acetate in hexane to give the title compound as colorless oil (4.34 g, 84.6%).

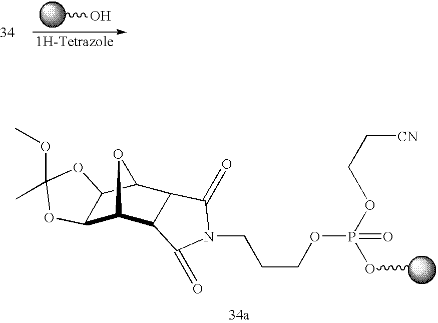

### Universal Support 34a

A suspension of hydroxy-terminated Tentagel (3.0 g) in MeCN (12 mL) is reacted with phosphoramidite 34 (0.2 M in MeCN; 1.0 mL) and 1H-tetrazole (0.45 M in MeCN; 1.0 mL) for 45 min. The solid phase is filtered off and treated with t-butyl hydroperoxide (10% in MeCN; 20 mL) for 30 min. The solid support is filtered off, washed with MeCN, and and capped with a mixture of Ac₂O/pyridine/N-methylimidazole/THF (10:10:10:70) at room temperature overnight. Finally, the solid support 34a is washed with MeCN and ethyl acetate and dried to give a loading of 50 to 60 μmol g⁻¹.

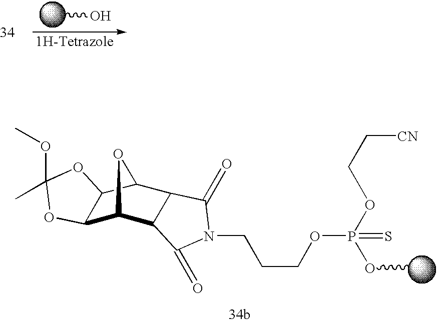

### Universal Support 34b

A suspension of hydroxy-terminated Tentagel (3.0 g) in MeCN (12 mL) is reacted with phosphoramidite 34 (0.2 M in MeCN; 1.0 mL) and 1H-tetrazole (0.45 M in MeCN; 1.0 mL) for 45 min. The solid phase is filtered off and treated with tetraethyl thiuram disulfide (0.1 M in MeCN; 20 mL) for 30 min. The solid support is filtered off, washed with MeCN, and and capped with a mixture of Ac₂O/pyridine/N-methylimidazole/THF (10:10:10:70) at room temperature overnight. Finally, the solid support 34b is washed with MeCN and ethyl acetate and dried to give a loading of 50 to 60 μmol g⁻¹.

### Example 15

### Universal Solid Support 40

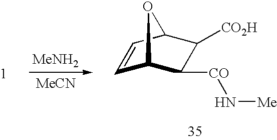

### Exo-N-methyl-7-oxabicyclo[2.2.1]hept-5-ene-3-carboxamide-2-carboxylic acid (35)

A solution of compound 1 (12.46 g, 75.0 mmol) in MeCN (150 mL) was treated with 40% aqueous methyamine (6.11 g, 78.7 mmol) in an ice-water bath for 45 min and at room temperature for 2 h. The precipitate was filtered off and washed on the filter with MeCN (3×50 mL). The precipitate was dried in vacuo to give ca. 98% pure compound 35 (10.24 g, 65.91%), which was used in the next synthetic step without any further purification.

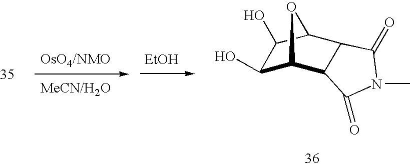

### Exo-N-methyl-5,6-dihydroxy-7-oxabicyclo[2.2.1]heptane-2,3-dicarboximide (36)

A solution of compound 35 (4.93 g, 25.0 mmol) and N-methylmorpholine-N-oxide (3.08 g, 26.3 mmol) in acetonitrile (20.0 mL) and water (7.5 mL) was treated with osmium tetroxide (6 mg, 0.025 mmol) in t-butanol (0.33 mL) for 1 h at 60-65° C. The reaction mixture was evaporated and co-evaporated with acetonitrile (2×50 mL). The residue was suspended in ethanol (45 mL) and boiled with a reflux condenser for 3 h. The mixture was diluted with ethanol (40 mL) and water (15 mL) and filtered through a short pad of silica gel. Silica gel was washed with 85% aqueous ethanol (75 mL), and the combined solution was evaporated. The solid residue was re-crystallized to give compound 36 (4.52 g, 84.8%) as an off-white solid.

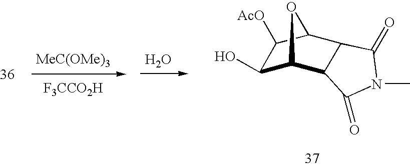

### Exo-N-methyl-5-hydroxy-6-acetoxy-7-oxabicyclo[2.2.1]heptane-2,3-dicarboximide (37)

A mixture of compound 36 (3.52 g, 16.5 mmol), trimethyl orthoacetate (2.17 g, 18.1 mmol), trifluoroacetic acid (38 mg), and acetonitrile (100 mL) was shaken overnight and treated with water (2 mL). The solvent was evaporated, and the solid residue was recrystallized to give compound 37 (3.92 g, 93.0%).

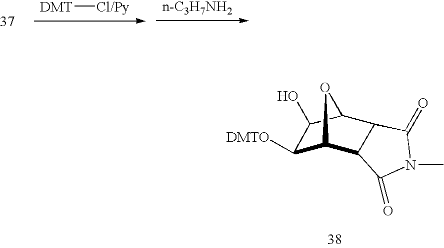

### Exo-N-methyl-5-hydroxy-6-(4,4′-dimethoxytrityloxy)-7-oxabicyclo[2.2.1]heptane-2,3-dicarboximide (38)

A mixture of compound 37 (3.83 g, 15.0 mmol), dimethoxytrityl chloride (5.33 g, 15.8 mmol), and pyridine (100 mL) was shaken overnight and treated with n-propylamine (2 mL) for 3 h. The solvent was evaporated, the residue was dissolved in ethyl acetate (200 mL) and washed with 5% aqueous NaHCO₃ (3×20 mL) followed by brine (20 mL). The organic phase was dried over Na₂SO₄ and evaporated. The residue was purified on a silica gel column eluting with a gradient of MeOH in CH₂Cl₂ to give compound 38 (7.88 g, 94.4%).

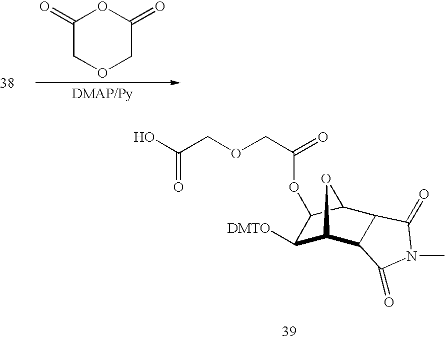

### Exo-N-methyl-5-(diglycolyloxy)-6-(4,4′-dimethoxytrityloxy)-7-oxabicyclo[2.2.1]heptane-2,3-dicarboximide (39)

A solution of compound 38 (12.25 g, 22.0 mmol), diglycolic anhydride (3.83 g, 33.0 mmol), and 4-dimethylaminopyridine (122 mg, 1.0 mmol) in pyridine (100 mL) was kept overnight. The solvent was evaporated, the residue was treated with ethyl acetate (200 mL) and 1 M aqueous triethylammonium acetate (20 mL). The organic solution was washed with 1 M aqueous triethylammonium acetate (4×20 mL), water (3×20 mL) dried over Na₂SO₄, and evaporated. The residue was dissolved in ethyl acetate (50 mL) and precipitated into hexane (500 mL). The precipitate was collected, washed with hexane, and dried in vacuo to give 39 (13.10 g, 90.5%) as an off-white solid.

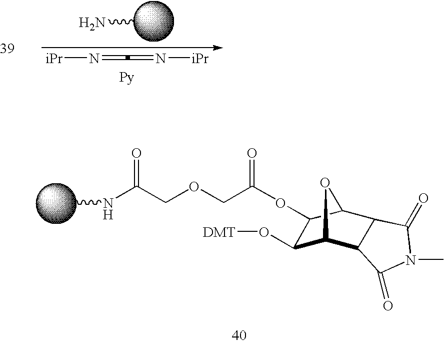

### Universal Solid Support 40

A mixture of compound 39 (658 mg, 1.0 mmol), long chain aminoalkyl controlled pore glass (4.27 g, 117 μmol gel, 0.5 mmol), N,N′-diisopropylcarbodiimide (189 mg, 1.5 mmol), and DMAP (12 mg, 0.1 mmol) in pyridine (20 mL) was shaken overnight. The solid phase was filtered, washed with pyridine (5×20 mL), and capped with a mixture of Ac₂O /pyridine/N-methylimidazole/THF (10:10:10:70; 40 mL) for 3 h. Finally, the solid phase was washed with MeCN and ethyl acetate and dried to give the solid support 40 loaded at 65.6 μmol g⁻¹.

### Example 16

### Universal Solid Support 46

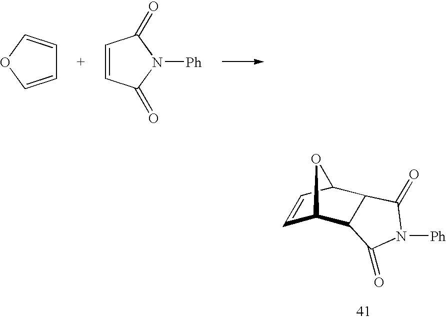

### N-Phenyl-tetrahydro-4,7-epoxyisobenzopyrrole-1,3-dione (41)

A solution of commercial furane (g, 100 mmol) and commercial N-phenyl-maleimide (g, 90 mmol) in anhydrous MeCN was refluxed overnight. The solvent was evaporated, and the residue was re-crystallized from hexane and toluene to give the title compound.

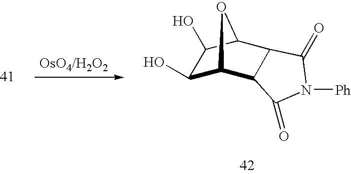

### N-phenyl-5,6-dihydroxy-hexahydro-4,7-epoxyisobenzopyrrole-1,3-dione (42)

A solution of compound 41 (12.06 g, 50 mmol) in hydrogen peroxide (30% aqueous, 2.56 g, 75 mmol), MeCN (50 mL), was treated with osmium tetroxide (2% aqueous, 12.7 mL, 1.0 mmol) for 4 h. The reaction mixture was evaporated, diluted with ethyl acetate (200 mL), washed with 5% aqueous NaHCO₃ saturated with brine (5×20 mL), dried over Na₂SO₄, and evaporated. The crystalline residue was re-crystallized from hexane and ethyl acetate to give pure 42.

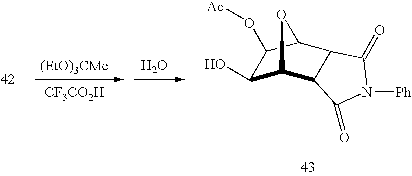

### N-phenyl-5-hydroxy-6-acetoxy-hexahydro-4,7-epoxyisobenzopyrrole-1,3-dione (43)

A solution of compound 42 (2.75 g, 10.0 mmol), triethyl orthoacetate (2.03 g, 12.59 mmol), and trifluoroacetic acid (0.023 g, 0.2 mmol) in DMF (50 mL) was kept overnight and then treated with water for 15 min (5 mL). The solvent was evaporated, and 5% aqueous NaHCO₃ (100 mL) is added. The mixture is extracted with ethyl acetate (200 mL). The organic phase was washed with 5% aqueous NaHCO₃ (5×20 mL), brine (3×20 mL), dried over Na₂SO₄, and evaporated. The title compound was isolated by column chromatography on silica gel, or it may be used in the next synthetic step without any further purification.

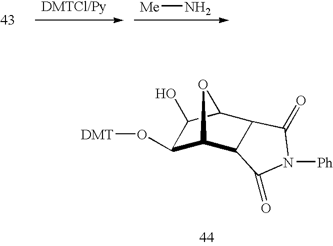

### N-phenyl-5-(4,4′-dimethoxytrityloxy)-6-acetoxy-hexahydro-4,7-epoxyisobenzopyrrole-1,3-dione (44)

A solution of compound 43 (3.17 g, 10 mmol) and 4,4′-dimethoxytrityl chloride (3.72 g, 11 mmol) in pyridine (50 mL) was kept overnight and then treated with methylamine (1 M in THF, 25 mL, 25 mmol) for 2 h. The solvent was evaporated, the residue is dissolved in ethyl acetate (200 mL) and washed with 5% aqueous NaHCO₃ (3×20 mL) and brine (50 mL). The organic phase was dried over Na₂SO₄ and evaporated. The residue was dissolved in ethyl acetate (20 mL) and precipitated into hexane (250 mL). The precipitate was collected, washed with hexane, and dried to give 44. Alternatively, the title compound may be isolated by chromatography on a silica gel column.

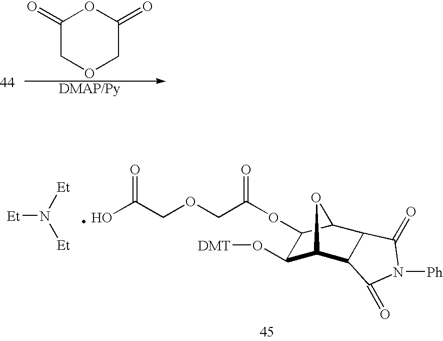

### Triethylammonium N-phenyl-5-(4,4′-dimethoxytrityloxy)-6-[[(carboxymethyl)oxy]acetoxy]-hexahydro-4,7-epoxyisobenzopyrrole-1,3-dione (45)

A solution of compound 44 (578 mg, 1.0 mmol), diglycolic anhydride (348 mg, 3.0 mmol), and 4-dimethylaminopyridine (122 mg, 1.0 mmol) in pyridine (50 mL) was kept overnight. The solvent was evaporated, the residue was treated with ethyl acetate (200 mL) and 1 M aqueous triethylammonium acetate (20 mL). The organic solution was washed with 1 M aqueous triethylammonium acetate (5×20 mL), water (3×20 mL) dried over Na₂SO₄, and evaporated. The residue was dissolved in ethyl acetate (20 mL) and precipitated into hexane (250 mL). The precipitate was collected, washed with hexane, and dried to give 45.

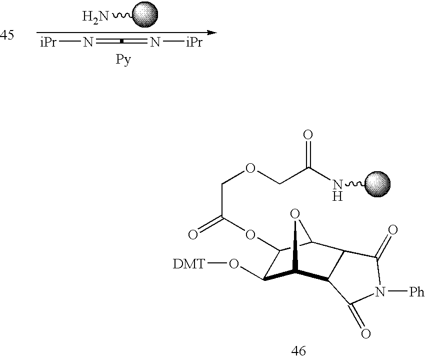

### Universal Solid Support 46

A mixture of compound 45 (795 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) was shaken overnight. The solid was filtered, washed with pyridine (5×10 mL) and capped with a mixture of Ac₂O/pyridine/N-methylimidazole/THF (10:10:10:70) for 3 h at room temperature. Finally, the solid support 46 was washed with MeCN and ethyl acetate and dried to give a loading of 36.6 μmol g⁻¹.

### Example 17

### Universal Solid Support 47

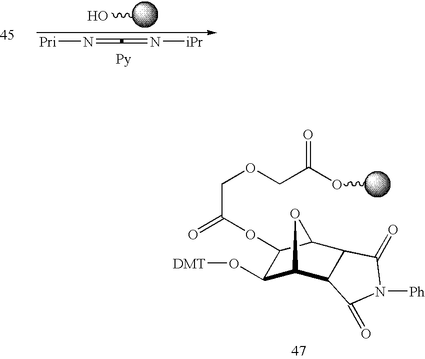

A mixture of compound 45 (795 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 47 is washed with MeCN and ethyl acetate and dried to give a loading of 17.5 μmol g⁻¹.

### Example 18

### Universal Solid Support 53

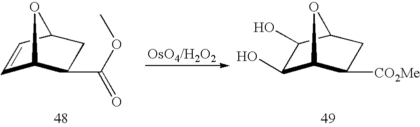

### Methyl (1α,2α,3α,4α,5α,6α)-5,6-dihydroxy-7-oxabicyclo[2.2.1]heptane-2-carboxylate (49)

Methyl 7-oxabicyclo[2.2.1]-5-heptene-2-carboxylate (48) is synthesized as described previously in Bridon, F. Tetrahedron Lett. 1982, 23, 5299-5302. A solution of 48, (7.71 g, 50 mmol) in hydrogen peroxide (30% aqueous, 2.56 g, 75 mmol), MeCN (50 mL), is treated with osmium tetroxide (2% aqueous, 12.7 mL, 1.0 mmol) for 4 h. The reaction mixture is evaporated, diluted with ethyl acetate (200 mL), washed with 5% aqueous NaHCO₃ saturated with brine (5×20 mL), dried over Na₂SO₄, and evaporated. The crystalline residue is re-crystallized from hexane and ethyl acetate to give pure 49.

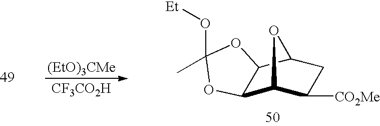

### Methyl 5,6-O-(1-ethoxyethylydene)-5,6-dioxy-7-oxabicyclo[2.2.1]heptane-2-carboxylate (50)

A solution of compound 49 (1.88 g, 10.0 mmol), triethyl orthoacetate (2.03 g, 12.5 mmol), and trifluoroacetic acid (0.023 g, 0.2 mmol) in MeCN (25 mL) is kept overnight and neutralized with ice-cold 5% aqueous NaHCO₃ (100 mL). The mixture is extracted with ethyl acetate (200 mL). The organic phase is washed with 5% aqueous NaHCO₃ (5×20 mL), brine (3×20 mL), dried over Na₂SO₄, and evaporated. The title compound may be isolated by column chromatography on silica gel, or it may be used in the next synthetic step without any further purification.

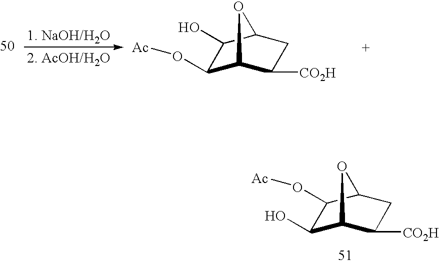

### 5-hydroxy-6-acetoxy-7-oxabicyclo[2.2.1]heptane-2,3-dicarboxylic acid (51)

A solution of compound 50 (488 mg, 2 mmol) and NaOH (100 mg, 2.5 mmol) in 80% aqueous MeOH (10 mL) is kept overnight and acidified by adding aqueous HCl (1 M, 3.0 mL, 3 mmol). The solvent is evaporated, 10% aqueous citric acid (20 mL) is added, and the product is extracted with ethyl acetate (5×100 mL). The organic solution is washed with water (20 mL), dried over Na₂SO₄, and evaporated. The residue is dissolved in ethyl acetate (20 mL) and precipitated into hexane (250 mL). The precipitate is collected, washed with hexane, and dried to give 51.

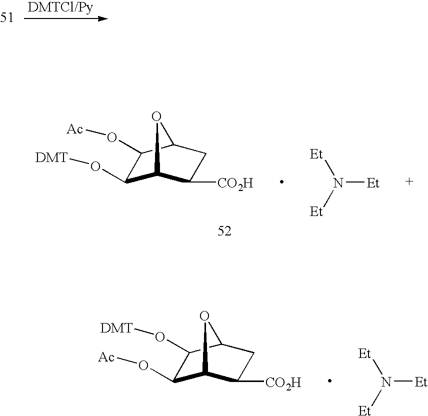

### Mixture of triethylammonium 5-(4,4′-dimethoxytrityloxy)-6-acetoxy-7-oxabicyclo[2.2.1]heptane-2-carboxylate and triethylammonium 5-acetoxy-6-(4,4′-dimethoxytrityloxy)-7-oxabicyclo[2.2.1]heptane-2-carboxylate (52)

A solution of compound 51 (432 mg, 2 mmol) and 4,4′-dimethoxytrityl chloride (845 mg, 2.5 mmol) in pyridine (10 mL) is kept overnight. The solvent is evaporated, the residue is treated with ethyl acetate (100 mL) and 1 M aqueous triethylammonium acetate (20 mL). The organic solution is washed with 1 M aqueous triethylammonium acetate (4×20 mL), water (4×20 mL), dried over Na₂SO₄, and evaporated. The residue is dissolved in ethyl acetate (20 mL) and precipitated into hexane (250 mL). The precipitate is collected, washed with hexane, and dried to give 52.

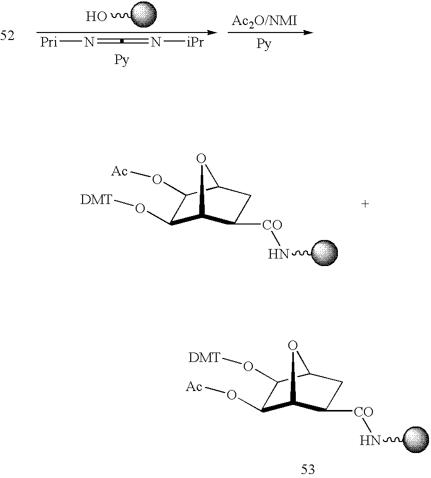

### Universal Solid Support 53

A mixture of compound 52 (620 mg, 1.0 mmol), amino alkyl controlled pore glass (2.50 g, 115 μmol g⁻¹), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10: 10:10:70) for 3 h at room temperature. Finally, the solid support 53 is washed with MeCN and ethyl acetate and dried to give a loading of 70 to 80 μmol g⁻¹.

### Example 19

### Universal Solid Support 54

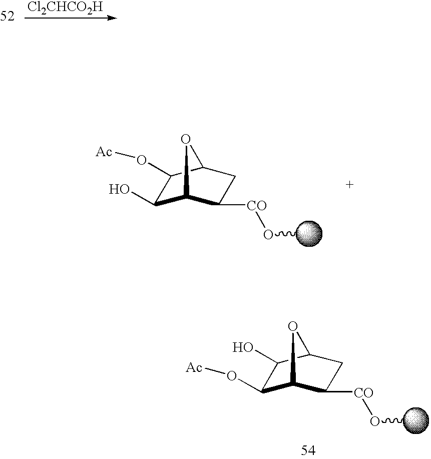

### Universal Solid Support 54

The solid support 53 (2.0 g) is washed with 3% dichloroacetic acid (20 mL) for 5 min followed by washing with pyridine (3×10 mL). Finally, the solid support 54 is washed with MeCN and dried.

### Example 20

### Universal Solid Support 57

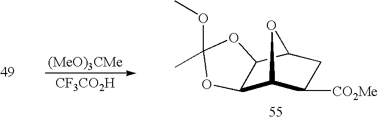

### Methyl 5,6-O-(1-methoxyethylydene)-5,6-dioxy-7-oxabicyclo[2.2.1]heptane-2carboxylate (55)

A solution of compound 49 (1.88 g, 10.0 mmol), trimethyl orthoacetate (1.50 g, 12.5 mmol), and trifluoroacetic acid (0.023 g, 0.2 mmol) in MeCN (25 mL) is kept overnight and neutralized with ice-cold 5% aqueous NaHCO₃ (100 mL). The mixture is extracted with ethyl acetate (200 mL). The organic phase is washed with 5% aqueous NaHCO₃ (5×20 mL), brine (3×20 mL), dried over Na₂SO₄, and evaporated. The title compound may be isolated by column chromatography on silica gel, or it may be used in the next synthetic step without any further purification.

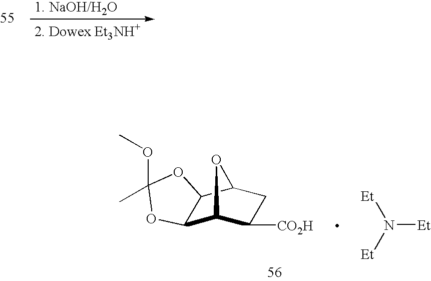

### Triethylammonium 5,6-O-(1-methoxyethylydene)-5,6-dioxy-7-oxabicyclo[2.2.1]heptane-2-carboxylate (56)

A solution of compound 55 (2.44 g, 10.0 mmol) and NaOH (0.60 g, 15 mmol) in 80% aqueous methanol (50 mL) is kept overnight and then passed through a Dowex 50WX4 column (Et₃NH⁺, 100 mL). The column is further washed with 80% aqueous MeOH (250 mL). The combined eluates are evaporated, co-evaporated with 5% triethylamine in methanol (3×50 mL), 5% triethylamine in toluene (5×50 mL). The viscous residue is dried in vacuo and used in the next synthetic step without any further purification.

Universal solid support 57.

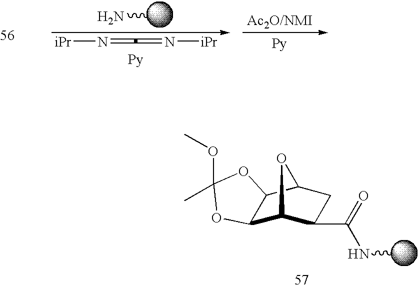

A mixture of compound 56 (331 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 57 is washed with MeCN and ethyl acetate and dried to give a loading of 70 to 80 μmol g⁻¹.

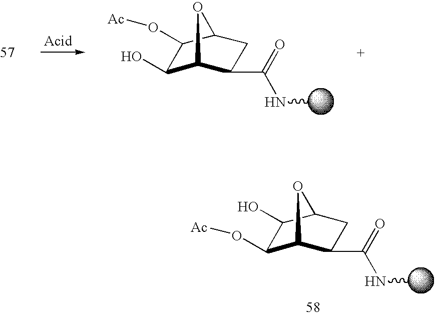

### Example 21

### Universal Solid Support 58

(a) The solid support 57 (500 mg) is treated with 80% aqueous acetic acid (4 mL) for 10 min. The suspension is filtered, and the solid support is washed with pyridine (3×10 mL). Finally, the solid support 58 is washed with MeCN and ethyl acetate and dried.

(b) The solid support 57 (1.00 g) is washed on a filter with 3% dichloroacetic acid in CH₂CI₂ (50 mL) for 5 min. The solid support 58 obtained is washed with MeCN (5×10 mL) and dried.

### Example 22

### Universal Solid Support 59

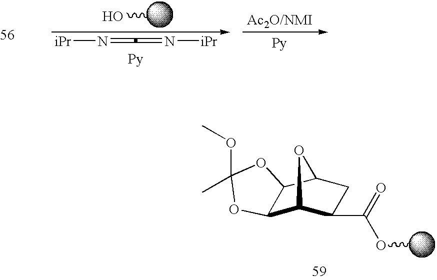

A mixture of compound 56 (331 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 59 is washed with MeCN and ethyl acetate and dried to give a loading of 70 to 80 μmol g⁻¹.

### Example 23

### Universal Solid Support 60

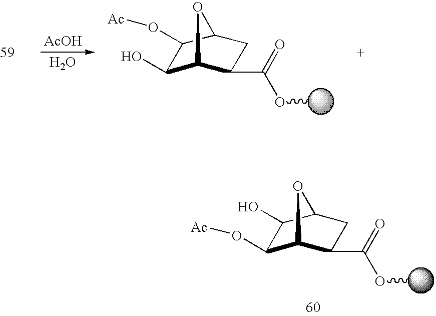

The solid support 59 (500 mg) is treated with 80% aqueous acetic acid (4 mL) for 10 min. The suspension is filtered, and the solid support is washed with pyridine (3×10 mL). Finally, the solid support 60 is washed with MeCN and ethyl acetate and dried.

### Example 24

### Universal Solid Support 65

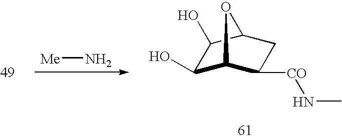

### N-methyl-5,6-dihydroxy-7-oxabicyclo[2.2.1]heptane-2-carboxamide (61)

A solution of compound 49 (1.88 g, 10.0 mmol) in THF (50 mL) is treated with metylamine (1 M in THF, 15 mL, 15 mmol) for 2 h. The solvent is evaporated, the crystalline precipitate is collected and, upon re-crystallization from toluene-ethyl acetate is dried in vacuo.

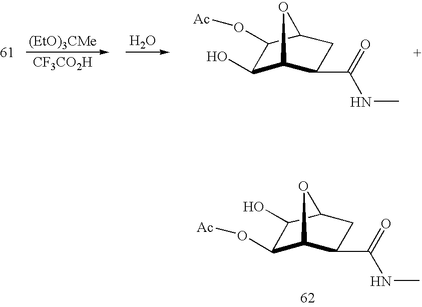

### Mixture of N-methyl-5-hydroxy-6-acetoxy-7-oxabicyclo[2.2. 1]heptane-2-carboxamide and N-methyl-5-acetoxy-6-hydroxy-7-oxabicyclo[2.2.1]heptane-2-carboxamide (62)

A solution of compound 61 (1.87 g, 10.0 mmol), triethylorthoacetate (2.03 g, 12.5 mmol), and trifluoroacetic acid (0.023 g, 0.2 mmol) in MeCN (50 mL) is kept overnight and then treated with water for 15 min (5 mL). The solvent is evaporated, and the residue is treated with ethyl acetate (200 mL). The crystalline precipitate is collected and, upon re-crystallization from toluene-ethyl acetate dried in vacuo.

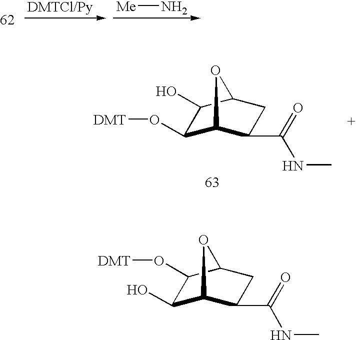

### Mixture of N-methyl-5-hydroxy-6-(4,4′-dimethoxytrityloxy)-7-oxabicyclo[2.2.1]heptane-2-carboxamide and N-methyl-5-(4,4′-dimethoxytrityloxy)-6-hydroxy-7-oxabicyclo[2.2.1]heptane-2-carboxamide (63)

A solution of compound 62 (2.29 g, 10 mmol) and 4,4′-dimethoxytrityl chloride (3.72 g, 11 mmol) in pyridine (50 mL) is kept overnight and then treated with methylamine (1 M in THF, 25 mL, 25 mmol) for 2 h. The solvent is evaporated, the residue is dissolved in ethyl acetate (200 mL) and washed with 5% aqueous NaHCO₃ (3×20 mL) and brine (50 mL). The organic phase is dried over Na₂SO₄ and evaporated. The residue is dissolved in ethyl acetate (20 mL) and precipitated into hexane (250 mL). The precipitate is collected, washed with hexane, and dried to give 63. Alternatively, the title compound may be isolated by chromatography on a silica gel column.

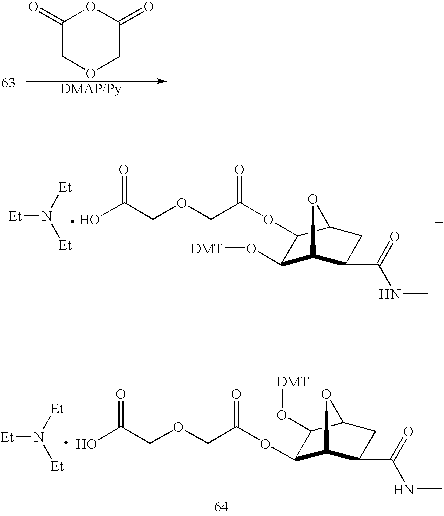

### Mixture of triethylammonium N-methyl-5-[[(carboxymethyl)oxy]acetoxy]-6-(4,4′-dimethoxytrityloxy)-7-oxabicyclo[2.2.1]heptane-2-carboxamide and triethylammonium N-methyl-5-(4,4,4′-dimethoxytrityloxy)-6-[[(carboxymethyl)oxy]acetoxy]-7-oxabicyclo[2.2.1]heptane-2-carboxamide (64)

A solution of compound 63 (490 mg, 1.0 mmol), diglycolic anhydride (348 mg, 3.0 mmol), and 4-dimethylaminopyridine (122 mg, 1.0 mmol) in pyridine (50 mL) is kept overnight. The solvent is evaporated, the residue is treated with ethyl acetate (200 mL) and 1 M aqueous triethylammonium acetate (20 mL). The organic solution is washed with 1 M aqueous triethylammonium acetate (5×20 mL), water (3×20 mL) dried over Na₂SO₄, and evaporated. The residue is dissolved in ethyl acetate (20 mL) and precipitated into hexane (250 mL). The precipitate is collected, washed with hexane, and dried to give 64.

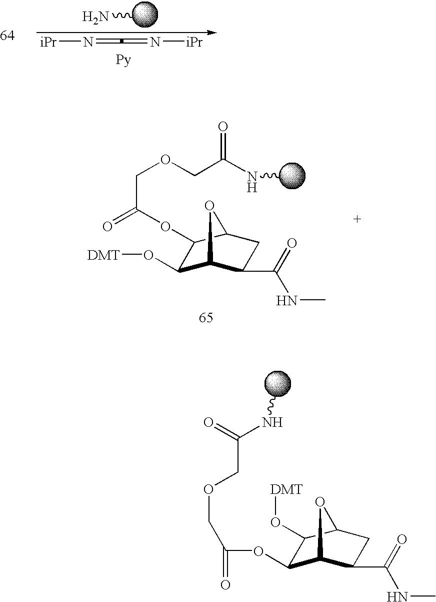

### Universal Solid Support 65

A mixture of compound 64 (707 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 65 is washed with MeCN and ethyl acetate and dried to give a loading of 70 to 80 μmol g⁻¹.

### Example 25

### Universal Solid Support 66

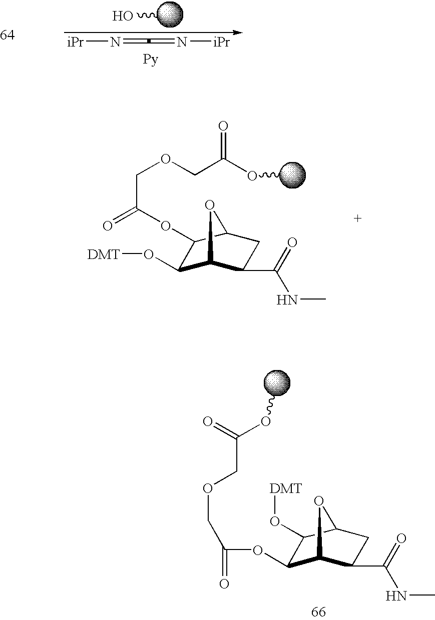

A mixture of compound 64 (707 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. 15 The solid is filtered, washed with pyridine (5×10 mL) and capped with a mixture of Ac₂O/pyridine/N-methylimidazole/THF (10:10:10:70) for 3 h at room temperature. Finally, the solid support 66 is washed with MeCN and ethyl acetate and dried to give a loading of 70 to 80 μmol g⁻¹.

### Example 26

### Universal Phosphoramidite Building Block 68 and Universal Solid Support 68a

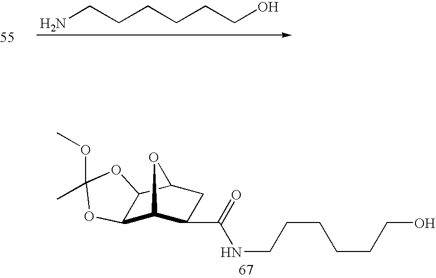

### N-(6-hydroxyhexyl)-5,6-dihydroxy-7-oxabicyclo[2.2.1]heptane-2-carboxamide (67)

A solution of compound 55 (2.44 g, 10.0 mmol) in THF (50 mL) is treated with 6-aminohexanol (1.76 g, 15 mmol) overnight. The solvent is evaporated, the residue is dissolved in ethyl acetate (200 mL) and washed with 5% aqueous NaHCO₃ (3×20 mL) and brine (50 mL). The 5 organic phase is dried over Na₂SO₄ and evaporated. The title compound is isolated by chromatography on a silica gel column.

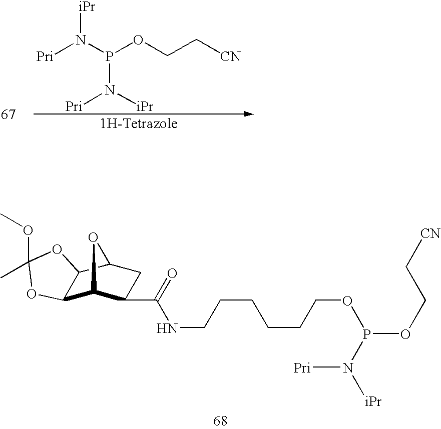

### 2-Cyanoethyl 6-[5,6-O-(1-methoxyethylydene)-5,6-dioxy-7-oxabicyclo[2.2.1]heptane-2-carboxamido]-hexyl N,N-diisopropylphosphoramidite (68)

A solution of compound 67 (3.29 g, 10.0 mmol) and 2-cyanoethyl N,N,N′,N′-tetraisopropylphosphorodiamidite (3.31 g, 10.5 mmol) in MeCN (20 mL) is treated with 1H-tetrazole (0.45 M in MeCN, 11.1 mL, 5.0 mmol) for 6 h. The reaction mixture is quenched with 5% aqueous NaHCO₃ (50 mL) and diluted with ethyl acetate (300 mL). The solution is washed with 5% aqueous NaHCO₃ (50 mL), brine (3×50 mL), dried over Na₂SO₄ and evaporated. The title compound is isolated on a silica gel column.

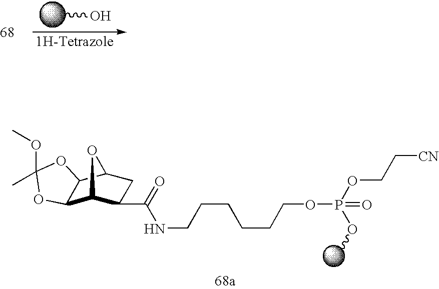

### Universal Support 68a

A suspension of hydroxy-terminated Tentagel (3.0 g) in MeCN (12 mL) is reacted with phosphoramidite 68 (0.2 M in MeCN; 1.0 mL) and 1H-tetrazole (0.45 M in MeCN; 1.0 mL) for 45 min. The solid phase is filtered off and treated with t-butyl hydroperoxide (10% in MeCN; 20 mL) 20 for 30 min. The solid support is filtered off, washed with MeCN, and and capped with a mixture of Ac₂O/pyridine/N-methylimidazole/THF (10:10:10:70) at room temperature overnight. Finally, the solid support 68a is washed with MeCN and ethyl acetate and dried to give a loading of 50 to 60 μmol g⁻¹.

### Example 27

### Universal Phosphoramidite Building Block 70 and Universal Support 70a

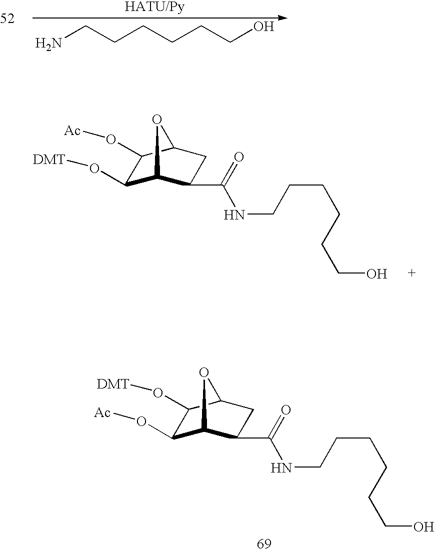

### Mixture of 6-[5-(4,4′-dimethoxytrityloxy)-6-acetoxy-7-oxabicyclo[2.2.1]heptane-2-carboxamido]-hexanol and 6-[5-acetoxy-6-(4,4′-dimethoxytrityloxy)-7-oxabicyclo[2.2.1]heptane-2-carboxamido]-hexanol (69)

A solution of compound 52 (6.20 g, 10.0 mmol) in MeCN (40 mL) and Py (10 mL) is treated with HATU (4.18 g, 11 mmol) for 15 min, and 6-aminohexanol (1.76 g, 15 mmol) is added. The reaction mixture is kept for 1 h and the solvent is evaporated. The residue is dissolved in ethyl acetate (200 mL) and washed with 5% aqueous NaHCO₃ (3×20 mL) and brine (50 mL). The organic phase is dried over Na₂SO₄ and evaporated. The title compound is isolated by chromatography on a silica gel column.

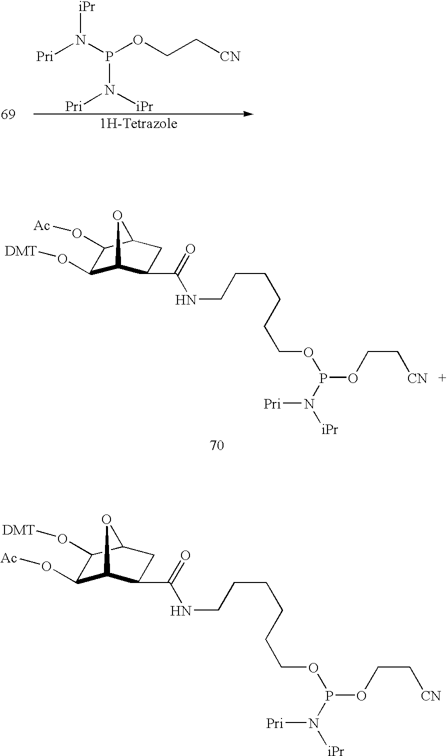

### Mixture of 2-cyanoethyl 6-[5-(4,4′-dimethoxytrityloxy)-6-acetoxy-7-oxabicyclo[2.2.1]heptane-2-carboxamido]-hexyl N,N′-diisopropylphosphoramidite and 2-cyanoethyl 6-[5-acetoxy-6-(4,4′-dimethoxytrityloxy)-7-oxabicyclo[2.2.1]heptane-2-carboxamido]-hexyl N,N′-diisopropylphosphoramidite (70).

A solution of compound 69 (6.04 g, 10.0 mmol) and 2-cyanoethyl N,N,N′,N′-tetraisopropylphosphorodiamidite (3.31 g, 10.5 mmol) in MeCN (20 mL) is treated with 1H-tetrazole (0.45 M in MeCN, 11.1 mL, 5.0 mmol) for 6 h. The reaction mixture is quenched with 5% aqueous NaHCO₃ (50 mL) and diluted with ethyl acetate (300 mL). The solution is washed with 5% aqueous NaHCO₃ (50 mL), brine (3×50 mL), dried over Na₂SO₄ and evaporated. The title compound is isolated on a silica gel column.

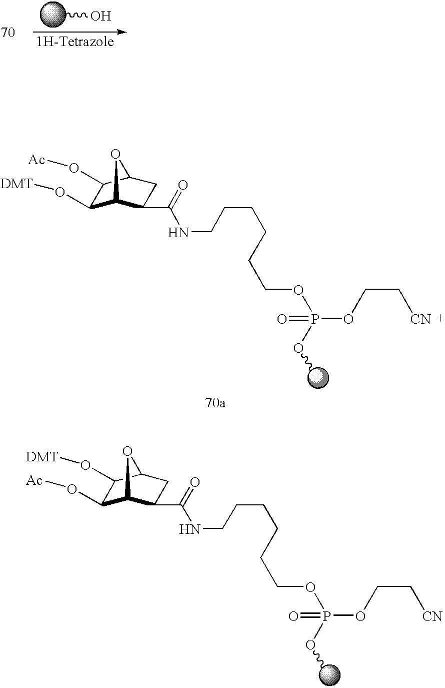

### Universal Support 70a

A suspension of hydroxy-terminated Tentagel (3.0 g) in MeCN (12 mL) is reacted with phosphoramidite 70 (0.2 M in MeCN; 1.0 mL) and 1H-tetrazole (0.45 M in MeCN; 1.0 mL) for 45 min. The solid phase is filtered off and treated with t-butyl hydroperoxide (10% in MeCN; 20 mL) for 30 min. The solid support is filtered off, washed with MeCN, and and capped with a mixture of Ac₂O/pyridine/N-methylimidazole/THF (10:10:10:70) at room temperature overnight. Finally, the solid support 73 is washed with MeCN and ethyl acetate and dried to give a loading of 50 to 60 μmol g⁻¹.

### Example 28

### Universal Solid Support 73

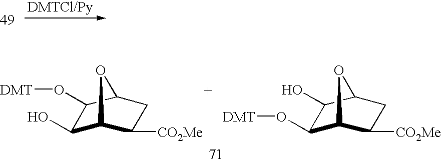

### Mixture of methyl (1α,2α,3α,4α,5α,6α)-5-hydroxy-6-(4,4′-dimethoxytrityloxy)-7-oxabicyclo[2.2.1]heptane-2-carboxylate and methyl (1α,2α,3α,4α,5α,6α)-6-hydroxy-5-(4,4′-dimethoxytrityloxy)-7-oxabicyclo[2.2.1]heptane-2-carboxylate (71)

A solution of compound 49 (1.88 g, 10 mmol) and 4,4′-dimethoxytrityl chloride (2.70 g, 8 mmol) in pyridine (50 mL) is kept overnight. The solvent is evaporated, the residue is treated with ethyl acetate (200 mL) and 5% aqueous NaHCO₃ (20 mL). The organic solution is washed with 5% aqueous NaHCO₃ (20 mL), dried over Na₂SO₄, and evaporated. The title compound is isolated by chromatography on a silica gel column.

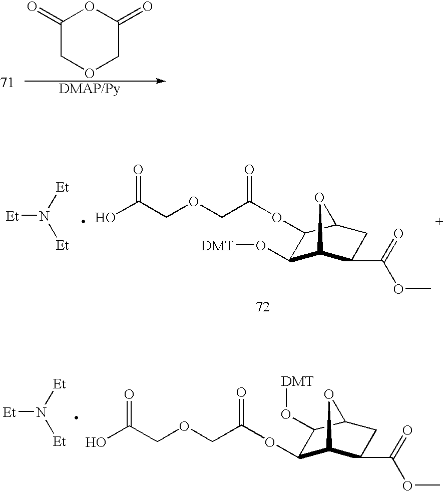

### Mixture of triethylammonium methyl N-methyl-5-[[(carboxymethyl)oxy]acetoxy]-6-(4,4′-dimethoxytrityloxy)-7-oxabicyclo[2.2.1]heptane-2-carboxylate and triethylammonium methyl N-methyl-5-(4,4′-dimethoxytrityloxy)-6-[[(carboxymethyl)oxy]acetoxy]-7-oxabicyclo[2.2.1]heptane-2-carboxylate(72)

A solution of compound 71 (491 mg, 1.0 mmol), diglycolic anhydride (348 mg, 3.0 mmol), and 4-dimethylaminopyridine (122 mg, 1.0 mmol) in pyridine (50 mL) is kept overnight. The solvent is evaporated, the residue is treated with ethyl acetate (200 mL) and 1 M aqueous triethylammonium acetate (20 mL). The organic solution is washed with 1 M aqueous triethylammonium acetate (5×20 mL), water (3×20 mL) dried over Na₂SO₄, and evaporated. The residue is dissolved in ethyl acetate (20 mL) and precipitated into hexane (250 mL). The precipitate is collected, washed with hexane, and dried to give 72.

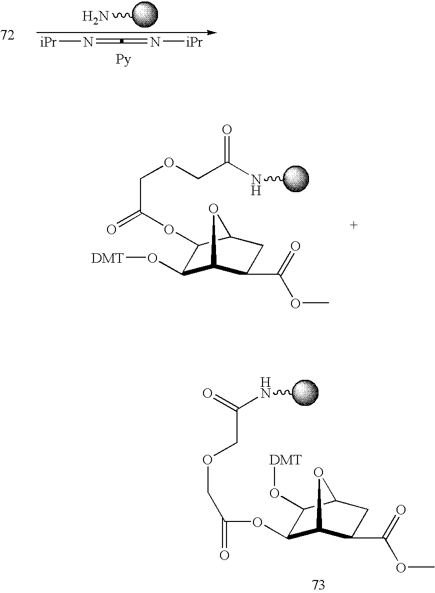

### Universal Solid Support 73

A mixture of compound 72 (708 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 73 is washed with MeCN and ethyl acetate and dried to give a loading of 70 to 80 μmol g⁻¹.

### Example 29

### Universal Solid Support 74

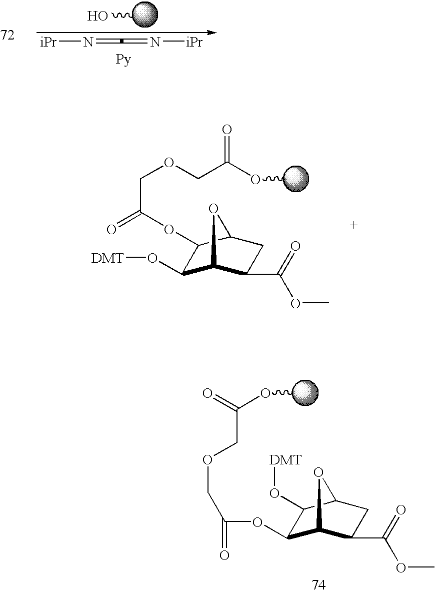

A mixture of compound 72 (708 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 74 is washed with MeCN and ethyl acetate and dried to give a loading of 70 to 80 μmol g⁻¹. acetate and dried.

### Example 30

### Universal Solid Support 75

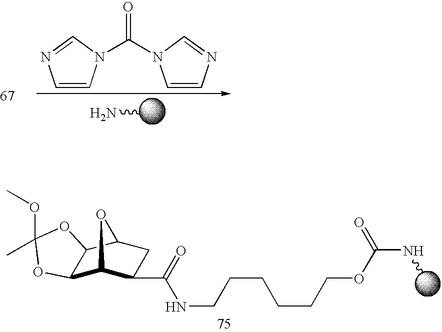

A solution of compound 67 (329 mg, 1.0 mmol) in Py (15 mL) is treated with carbonyldiimidazole (162 mg, 1.0 mmol) for 2 h. Long chain aminoalkyl controlled pore glass (2667 mg, 0.5 mmol) is added and the suspension is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 75 is washed with MeCN and ethyl acetate and dried to give a loading of 70 to 80 μmol g⁻¹.

### Example 31

### Universal Solid Support 76

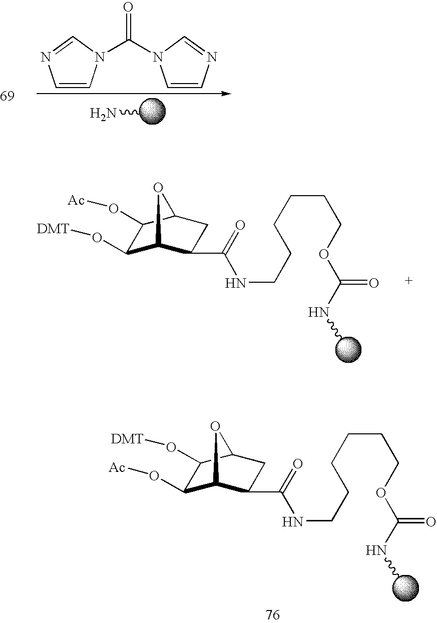

A solution of compound 69 (618 mg, 1.0 mmol) in Py (15 mL) is treated with carbonyldiimidazole (162 mg, 1.0 mmol) for 2 h. Long chain aminoalkyl controlled pore glass (2667 mg, 0.5 mmol) is added and the suspension is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 76 is washed with MeCN and ethyl acetate and dried to give a loading of 70 to 80 μmol g⁻¹.

### Example 32

### Universal Solid Support 78

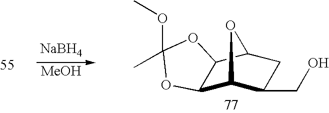

### 2,3-O-(1-methoxyethylydene)-2,3-dioxy-5-hydroxymethyl-7-oxabicyclo[2.2.1]heptane (77)

A solution of compound 55 (2.44 g, 10.0 mmol) in MeOH (25 mL) is treated with NaBH₄ (760 mg, 20 mmol) overnight at 50° C. and evaporated. The mixture is extracted with ethyl acetate (200 mL) and 5% aqueous NaHCO₃ (25 mL). The organic phase is washed with 5% aqueous NaHCO₃ (5×20 mL) saturated with brine, dried over Na₂SO₄, and evaporated. The title compound may be isolated by column chromatography on silica gel, or it may be used in the next synthetic step without any further purification.

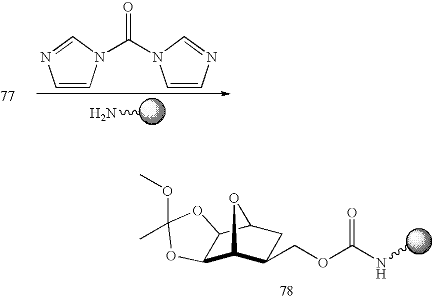

### Universal Solid Support 78

A solution of compound 77 (216 mg, 1.0 mmol) in Py (15 mL) is treated with carbonyldiimidazole (162 mg, 1.0 mmol) for 2 h. Long chain aminoalkyl controlled pore glass (2667 mg, 0.5 mmol) is added and the suspension is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 78 is washed with MeCN and ethyl acetate and dried to give a loading of 70 to 80 μmol g⁻¹.

### Example 33

### Universal Solid Support 83

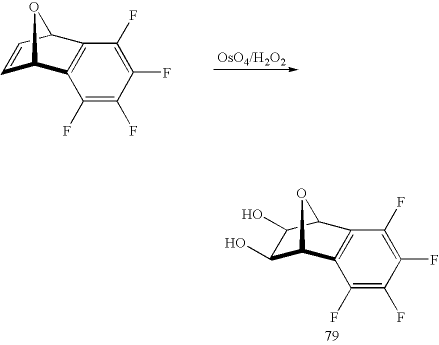

### 2,3-dihydroxy-9-oxa-5,6,7,8-tetrafluoro-2,3-dihydrobenzonorbornene (79)

A solution of 9-oxa-5,6,7,8-tetrafluorobenzonorbornadiene (10.81 g, 50 mmol, prepared as described in Caster, K. C.; Keck, C. G.; Walls, R. D. J. Org. Chem. 2001, 66, 2932-2936) in hydrogen peroxide (30% aqueous, 2.56 g, 75 mmol), MeCN (50 mL), is treated with osmium tetroxide (2% aqueous, 12.7 mL, 1.0 mmol) for 4 h. The reaction mixture is evaporated, diluted with ethyl acetate (200 mL), washed with 5% aqueous NaHCO₃ saturated with brine (5×20 mL), dried over Na₂SO₄, and evaporated. The residue is purified by column chromatography on silica gel to give pure 79.

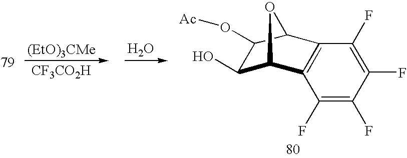

### 2-acetoxy-3-hydroxy-9-oxa-5,6,7,8-tetrafluoro-2,3-dihydrobenzonorbornene (80)

A solution of compound 79 (2.75 g, 10.0 mmol), triethyl orthoacetate (2.03 g, 12.59 mmol), and trifluoroacetic acid (0.023 g, 0.2 mmol) in DMF (50 mL) is kept overnight and then treated with water for 15 min (5 mL). The solvent is evaporated, and 5% aqueous NaHCO₃ (100 mL) is added. The mixture is extracted with ethyl acetate (200 mL). The organic phase is washed with 5% aqueous NaHCO₃ (5×20 mL), brine (3×20 mL), dried over Na₂SO₄, and evaporated. The title compound may be isolated by column chromatography on silica gel, or it may be used in the next synthetic step without any further purification.

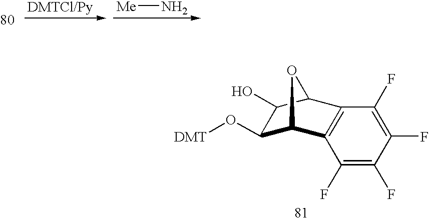

### 2-(4,4′dimethoxytrityl)-3-hydroxy-9-oxa-5,6,7,8-tetrafluoro-2,3-dihydrobenzo norbornene (81)

A solution of compound 80 (2.50 g, 10 mmol) and 4,4′-dimethoxytrityl chloride (3.72 g, 11 mmol) in pyridine (50 mL) is kept overnight and then treated with methylamine (1 M in THF, 25 mL, 25 mmol) for 2 h. The solvent is evaporated, the residue is dissolved in ethyl acetate (200 mL) and washed with 5% aqueous NaHCO₃ (3×20 mL) and brine (50 mL). The organic phase is dried over Na₂SO₄ and evaporated. The residue is dissolved in ethyl acetate (20 mL) and precipitated into hexane (250 mL). The precipitate is collected, washed with hexane, and dried to give 81. Alternatively, the title compound may be isolated by chromatography on a silica gel column.

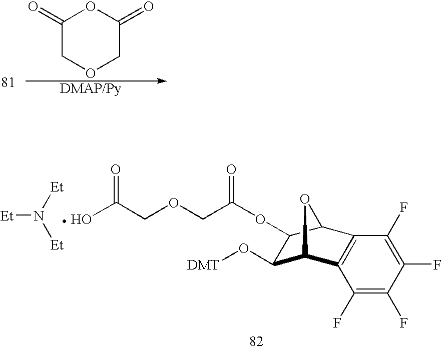

### Triethylammonium 2-(4,4′dimethoxytrityl)-3-[[(carboxymethyl)oxy]acetoxy]-9-oxa-5,6,7,8-tetrafluoro-2,3-dihydrobenzo norbornene (82)

A solution of compound 81 (553 mg, 1.0 mmol), diglycolic anhydride (348 mg, 3.0 mmol), and 4-dimethylaminopyridine (122 mg, 1.0 mmol) in pyridine (50 mL) is kept overnight. The solvent is evaporated, the residue is treated with ethyl acetate (200 mL) and 1 M aqueous triethylammonium acetate (20 mL). The organic solution is washed with 1 M aqueous triethylammonium acetate (5×20 mL), water (3×20 mL) dried over Na₂SO₄, and evaporated. The residue is dissolved in ethyl acetate (20 mL) and precipitated into hexane (250 mL). The precipitate is collected, washed with hexane, and dried to give 82.

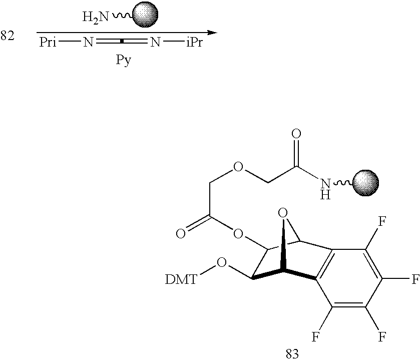

### Universal Solid Support 83

A mixture of compound 82 (770 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 83 is washed with MeCN and ethyl acetate and dried to give a loading of 70 to 80 μmol g⁻¹.

### Example 34

### Universal Solid Support 84

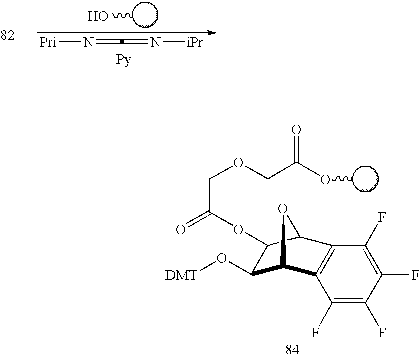

### Universal Solid Support 84

A mixture of compound 82 (770 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 84 is washed with MeCN and ethyl acetate and dried to give a loading of 70 to 80 μmol g⁻¹.

### Example 35

### Universal Solid Support 89

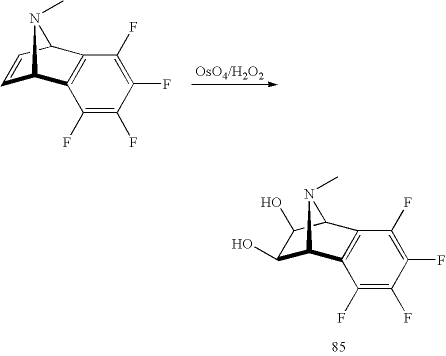

### 2,3-dihydroxy-9-methyl-9-aza-5,6,7,8-tetrafluoro-2,3-dihydrobenzonorbornene (85)

A solution of 9-methyl-9-aza-5,6,7,8-tetrafluorobenzonorbornadiene (11.46 g, 50 mmol, prepared as described in Caster, K. C.; Keck, C. G.; Walls, R. D. J. Org. Chem. 2001, 66, 2932-2936) in hydrogen peroxide (30% aqueous, 2.56 g, 75 mmol), MeCN (50 mL), is treated with osmium tetroxide (2% aqueous, 12.7 mL, 1.0 mmol) for 4 h. The reaction mixture is evaporated, diluted with ethyl acetate (200 mL), washed with 5% aqueous NaHCO₃ saturated with brine (5×20 mL), dried over Na₂SO₄, and evaporated. The residue is purified by column chromatography on silica gel to give pure 85.

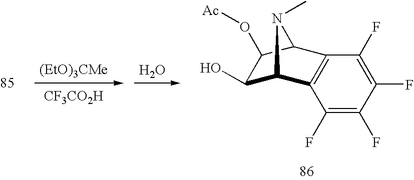

### 2-acetoxy-3-hydroxy-9-methyl-9-aza-5,6,7,8-tetrafluoro-2,3-dihydrobenzonorbornene (86)

A solution of compound 85 (2.88 g, 10.0 mmol), triethyl orthoacetate (2.03 g, 12.59 mmol), and trifluoroacetic acid (0.023 g, 0.2 mmol) in DMF (50 mL) is kept overnight and then treated with water for 15 min (5 mL). The solvent is evaporated, and 5% aqueous NaHCO₃ (100 mL) is added. The mixture is extracted with ethyl acetate (200 mL). The organic phase is washed with 5% aqueous NaHCO₃ (5×20 mL), brine (3×20 mL), dried over Na₂SO₄, and evaporated. The title compound may be isolated by column chromatography on silica gel, or it may be used in the next synthetic step without any further purification.

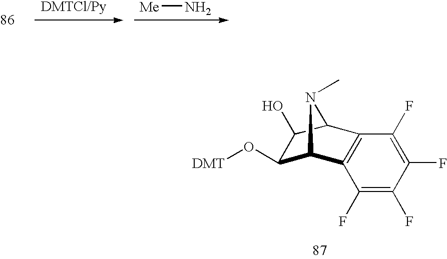

### 2-(4,4′dimethoxytrityl)-3-hydroxy-9-methyl-9-aza-5,6,7,8-tetrafluoro-2,3-dihydrobenzo norbornene (87)

A solution of compound 86 (2.63 g, 10 mmol) and 4,4′-dimethoxytrityl chloride (3.72 g, 11 mmol) in pyridine (50 mL) is kept overnight and then treated with methylamine (1 M in THF, 25 mL, 25 mmol) for 2 h. The solvent is evaporated, the residue is dissolved in ethyl acetate (200 mL) and washed with 5% aqueous NaHCO₃ (3×20 mL) and brine (50 mL). The organic phase is dried over Na₂SO₄ and evaporated. The residue is dissolved in ethyl acetate (20 mL) and precipitated into hexane (250 mL). The precipitate is collected, washed with hexane, and dried to give 87. Alternatively, the title compound may be isolated by chromatography on a silica gel column.

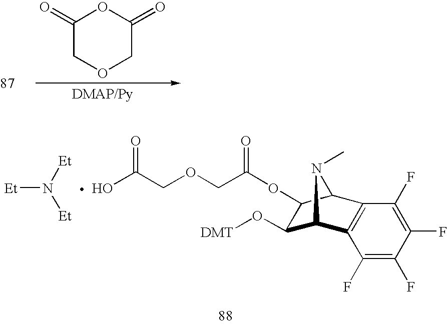

### Triethylammonium 2-(4,4′dimethoxytrityl)-3-[[(carboxymethyl)oxy]acetoxy]-9-methyl-9-aza-5,6,7,8-tetrafluoro-2,3-dihydrobenzo norbornene (88)

A solution of compound 87 (566 mg, 1.0 mmol), diglycolic anhydride (348 mg, 3.0 mmol), and 4-dimethylaminopyridine (122 mg, 1.0 mmol) in pyridine (50 mL) is kept overnight. The solvent is evaporated, the residue is treated with ethyl acetate (200 mL) and 1 M aqueous triethylammonium acetate (20 mL). The organic solution is washed with 1 M aqueous triethylammonium acetate (5×20 mL), water (3×20 mL) dried over Na₂SO₄, and evaporated. The residue is dissolved in ethyl acetate (20 mL) and precipitated into hexane (250 mL). The precipitate is collected, washed with hexane, and dried to give 88.

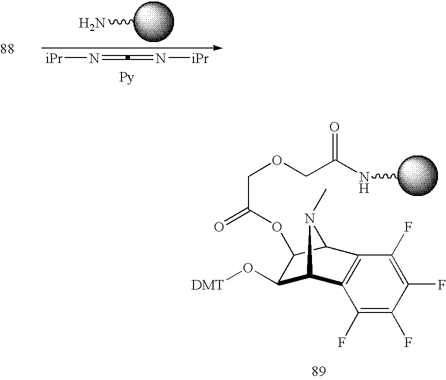

### Universal Solid Support 89

A mixture of compound 88 (783 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 89 is washed with MeCN and ethyl acetate and dried to give a loading of 70 to 80 μmol g⁻¹ as determined by the standard DMT assay.

### Example 36

### Universal Solid Support 90

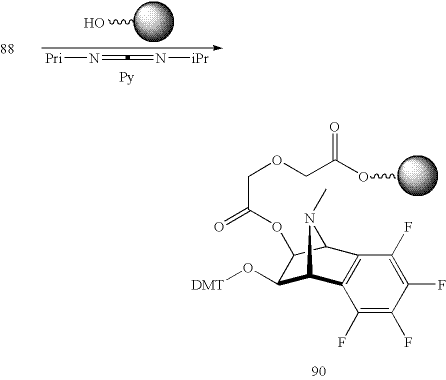

A mixture of compound 88 (783 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 90 is washed with MeCN and ethyl acetate and dried to give a loading of 70 to 80 μmol g-1 as determined by the standard DMT assay.

### Example 37

### Universal Solid Support 96

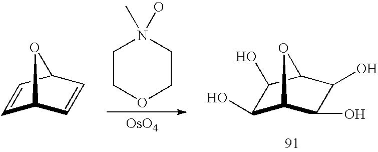

### (1α,2α,3α,4α,5α,6α)-2,3,5,6-tetrahydroxy-7-oxabicyclo[2.2.1]heptane (91)

A solution of commercial 7-oxanorbornadiene, (9.41 g, 100 mmol) and N-methylmorpholine N-oxide (24.60 g, 210 mmol) in MeCN (50 mL) is treated with osmium tetroxide (127 mg, 0.5 mmol) in t-butanol (5.1 mL) overnight. The reaction mixture is evaporated, co-evaporated with toluene (5×100 mL) and used in the next step without any further purification.

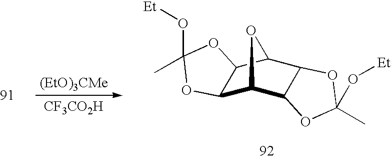

### (1α,2α,3α,4α,5α,6α)-2,3-O-5,6-O-bis(1-ethoxyethylydene)-2,3,5,6-tetraoxy-7-oxabicyclo[2.2.1]heptane (92)

A solution of compound 91 (1.62 g, 10.0 mmol), triethyl orthoacetate (4.06 g, 25 mmol), and trifluoroacetic acid (0.023 g, 0.2 mmol) in dioxane (50 mL) is kept overnight and neutralized with 5% aqueous NaHCO₃ (20 mL).The solvent is evaporated, the residue is dissolved in ethyl acetate (200 mL) and washed with 5% aqueous NaHCO₃ (3×20 mL) and brine (50 mL). The organic phase is dried over Na₂SO₄ and evaporated. The title compound is isolated by chromatography on a silica gel column.

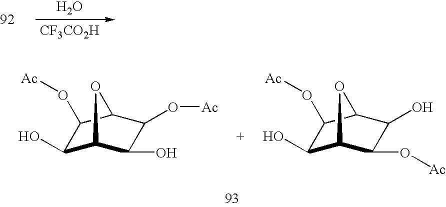

### Mixture of (1α,2α,3α,4α,5α,6α)-1,6-dihydroxy-2,5-diacetoxy-7-oxabicyclo[2.2.1]heptane and (1α,2α,3α,4α,5α,6α)-1,5-dihydroxy-2,6-diacetoxy-7-oxabicyclo[2.2.1]heptane (93)

A solution of compound 92 (3.02 g, 10.0 mmol) and trifluoroacetic acid (0.023 g, 0.2 mmol) in 80% aqueous MeOH (50 mL) is kept for 30 min. The reaction mixture is evaporated, co-evaporated with toluene (5×100 mL) and used in the next step without any further purification.

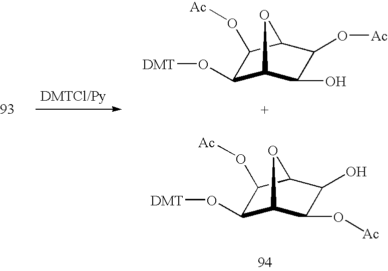

### Mixture of (1α,2α,3α,4α,5α,6α)-1-hydroxy-6-(4,4′-dimethoxytrityloxy)-2,5-diaxetoxy-7-oxabicyclo[2.2.1]heptane and (1α,2α,3α,4α,5α,6α)-1-hydroxy-5-(4,4′-dimethoxytrityloxy)-2,6-diaxetoxy-7-oxabicyclo[2.2.1]heptane (94)

A solution of compound 93 (2.46 g, 10 mmol) and 4,4′-dimethoxytrityl chloride (2.70 g, 8 mmol) in pyridine (50 mL) is kept overnight. The solvent is evaporated, the residue is treated with ethyl acetate (200 mL) and 5% aqueous NaHCO₃ (20 mL). The organic solution is washed with 5% aqueous NaHCO₃ (20 mL), dried over Na₂SO₄, and evaporated. The title compound is isolated by chromatography on a silica gel column.

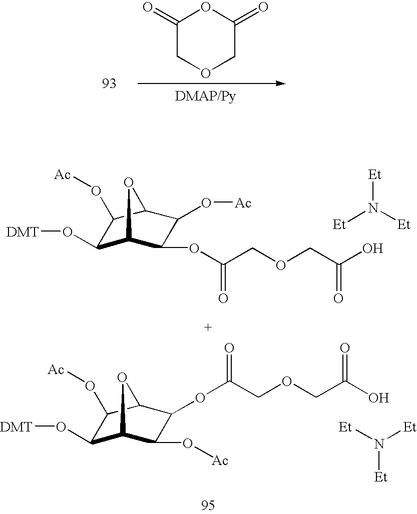

### Mixture of (1α,2α,3α,4α,5α,6α)-1-[[(carboxymethyl)oxy]acetoxy]-6-(4,4′-dimethoxytrityloxy)-2,5-diacetoxy-7-oxabicyclo[2.2.1]heptane and (1α,2α,3α,4α,5α,6α)-1-[[(carboxymethyl)oxy]acetoxy]-5-(4,4′-dimethoxytrityloxy)-2,6-diacetoxy-7-oxabicyclo[2.2.1]heptane (95)

A solution of compound 94 (549 mg, 1.0 mmol), diglycolic anhydride (348 mg, 3.0 mmol), and 4-dimethylaminopyridine (122 mg, 1.0 mmol) in pyridine (50 mL) is kept overnight. The solvent is evaporated, the residue is treated with ethyl acetate (200 mL) and 1 M aqueous triethylammonium acetate (20 mL). The organic solution is washed with 1 M aqueous triethylammonium acetate (5×20 mL), water (3×20 mL) dried over Na₂SO₄, and evaporated. The residue is dissolved in ethyl acetate (20 mL) and precipitated into hexane (250 mL). The precipitate is collected, washed with hexane, and dried to give 95.

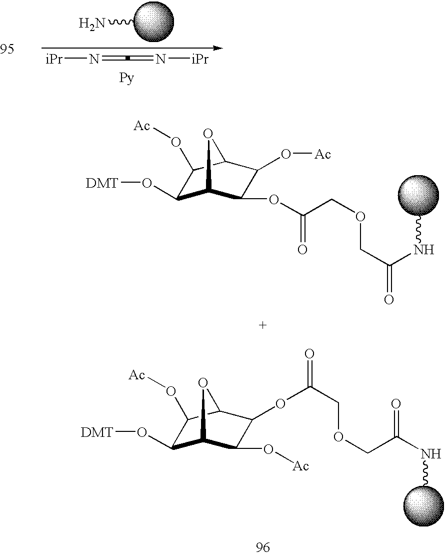

### Universal Solid Support 96

A mixture of compound 95 (766 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 96 is washed with MeCN and ethyl acetate and dried to give a loading of 70 to 80 μmol g⁻¹.

### Example 38

### Universal Solid Support 97

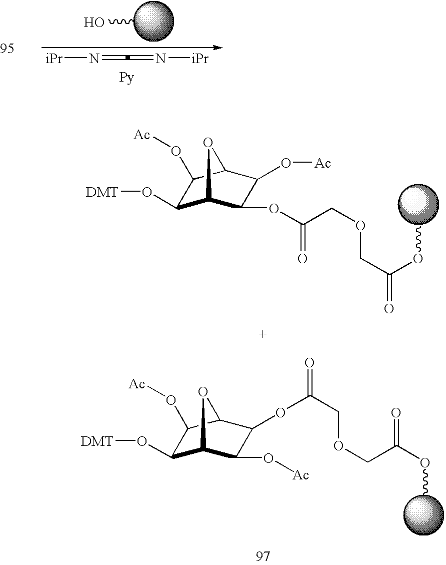

A mixture of compound 95 (766 mg, 1.0 mmol), PS-PEG solid support (2500 mg, 0.5 mmol), and N,N′-diisopropylcarbodiimide (252 mg, 2.0 mmol) in pyridine (20 mL) is shaken overnight.

The solid is filtered, washed with pyridine (5×10 mL) and capped with a mixture of Ac₂O/pyridine/N-methylimidazole/THF (10:10:10:70) for 3 h at room temperature. Finally, the solid support 97 is washed with MeCN and ethyl acetate and dried to give a loading of 70 to 80 μmol g⁻¹.

### Example 39

### Universal Solid Support 99

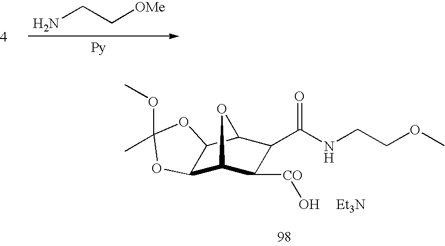

### Triethylammonium (1α,2α,3α,4α,5α,6α)-N-(2-methoxyethyl)-5,6-O-( 1-methoxyethylydene)-5,6-dioxy-3-carbamoyl-7-oxabicyclo[2.2.1]heptane-2-carboxylate (98)

Compound 4 (256 mg, 1.0 mmol) is treated with 2-methoxyethylamine (75 mg, 1.0 mmol) and pyridine (5 mL) for 1 h at room temperature. The solvent is evaporated, the residue is treated with ethyl acetate (200 mL) and 1 M aqueous triethylammonium acetate (20 mL). The organic solution is washed with 1 M aqueous triethylammonium acetate (5×20 mL), water (3×20 mL) dried over Na₂SO₄, and evaporated. The residue is dissolved in ethyl acetate (20 mL) and precipitated into hexane (250 mL). The precipitate is collected, washed with hexane, and dried to give 98.

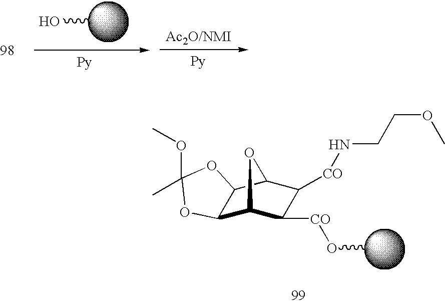

### Universal Solid Support 99

PS-PEG solid support (2500 mg, 0.5 mmol) is gently shaken with compound 152 (433 mg, 1.0 mmol) in pyridine (10 mL) overnight. The suspension is filtered, and the solid support is washed with pyridine (3×20 mL). The solid support is additionally washed with ethyl acetate, dried, and capped by treating with a mixture of Ac₂O/pyridine/N-methylimidazole/THF (10:10: 10:70) for 3 h at room temperature. Finally, the solid support 154 with a loading of 70 to 80 μmol g⁻¹ is washed with MeCN and ethyl acetate and dried.

### Example 40

### Universal Solid Support 105

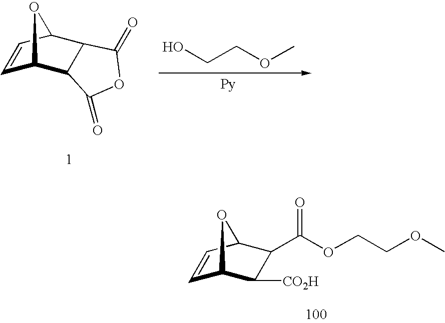

### Mono(2-methoxyethyl) (1α,2α,3α,4α,5α,6α)-7-oxabicyclo[2.2.1]hept-5-ne-2,3-dixcarboxylate (100)

A solution of commercial compound 1, (8.31 g, 50.0 mmol) and 4-dimethylaminopyridine (183 mg, 1.5 mmol) in 2-methoxyethanol (5.70 g, 75.0 mmol), triethylamine (5.06 g, 50 mmol), and dioxane (50 mL) is kept overnight and evaporated. The residue is dissolved in water (50 mL) and acidified with conc. aqueous HCl. The product is extracted with ethyl acetate, dried over Na₂SO₄, and evaporated to give practically pure 100.

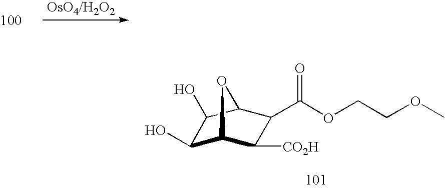

### 2-methoxyethyl (1α,2α,3α,4α,5α,6α)-5,6-dihydroxy-7-oxabicyclo[2.2.1]heptane-2-carboxylate-3-carboxylic acid (101)

A solution of compound 100 (13.31 g, 55 mmol) in hydrogen peroxide (30% aqueous, 2.97 g, 87.2 mmol), acetone (72.5 mL), ether (18.1 mL), and t-butanol (6.2 mL) was treated with osmium tetroxide (249 mg, 0.98 mmol) in t-butanol (10.0 mL) for 4 days at 28-30° C. The reaction mixture was treated with ether (90 mL) and kept at 4° C. for 1 h. The precipitate was filtered off, washed with ether and dried to give pure 101.

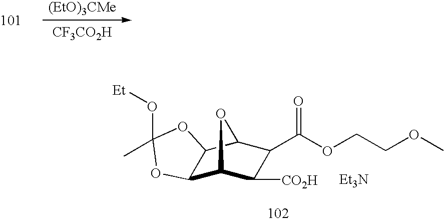

### Triethylammonium 2-methoxyethyl (1α,2α,3α,4α,5α,6α)-5,6-O-(1-ethoxyethylydene)-5,6-dioxy-7-oxabicyclo[2.2.1]heptane-2,3-dicarboxylate (102)

A solution of compound 101 (2.76 g, 10.0 mmol), triethyl orthoacetate (2.03 g, 12.5 mmol), and trifluoroacetic acid (0.023 g, 0.2 mmol) in DMF (50 mL) is kept overnight and neutralized with triethylamine (5 mL). The solvent is evaporated, and the residue is treated with ether (200 mL). The viscous precipitate is dried in vacuo and used in the next synthetic step without any further purification.

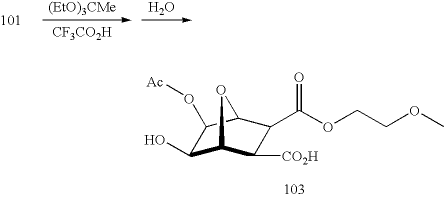

### 2-Methoxyethyl (1α,2α,3α,4α,5α,6α)-5-hydroxy-6-acetoxy-7-oxabicyclo[2.2.1]heptane-2,3-dicarboxylate (103)

A solution of compound 101 (2.76 g, 10.0 mmol), triethyl orthoacetate (2.03 g, 12.59 mmol), and trifluoroacetic acid (0.023 g, 0.2 mmol) in DMF (50 mL) is kept overnight and then treated with water for 15 min (5 mL). The solvent is evaporated, and the residue is treated with ether (200 mL). The viscous precipitate is dried in vacuo and used in the next synthetic step without any further purification.

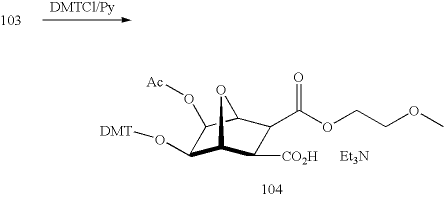

### Triethylammonium 2-methoxyethyl (1α,2α,3α,4α,5α,6α)-5-(4,4′-dimethoxytrityloxy)-6-acetoxy-7-oxabicyclo[2.2.1]heptane-2,3-dicarboxylate (104)

A solution of compound 103 (636 mg, 2 mmol) and 4,4′-dimethoxytrityl chloride (845 mg, 2.5 mmol) in pyridine (10 mL) is kept overnight. The solvent is evaporated, the residue is treated with ethyl acetate (100 mL) and 1 M aqueous triethylammonium acetate (20 mL). The organic solution is washed with 1 M aqueous triethylammonium acetate (4×20 mL), water (4×20 mL), dried over Na₂SO₄, and evaporated. The residue is dissolved in ethyl acetate (20 mL) and precipitated into hexane (250 mL). The precipitate is collected, washed with hexane, and dried to give 104.

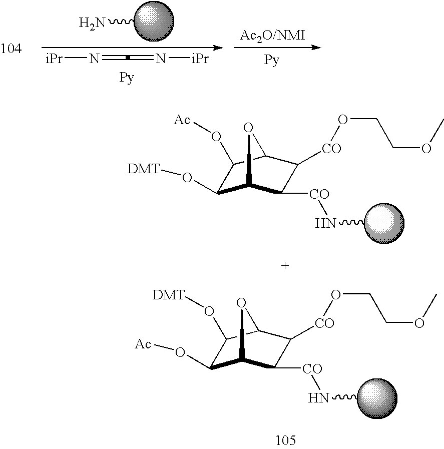

### Universal Solid Support 105

Aminoalkyl controlled pore glass (2667 mg, 0.5 mmol) is gently shaken with compound 104 (722 mg, 1.0 mmol) in pyridine (10 mL) overnight. The suspension is filtered, and the solid support is washed with pyridine (3×20 mL). The solid support is washed with MeCN (5×10 mL) and capped with a mixture of Ac₂O/pyridine/N-methylimidazole/THF (10: 10: 10:70) for 3 h at room temperature. Finally, the solid support 105 with a loading of 70 to 80 μmol g⁻¹ is washed with MeCN and ethyl acetate and dried.

### Example 41

### Universal Solid Support 107

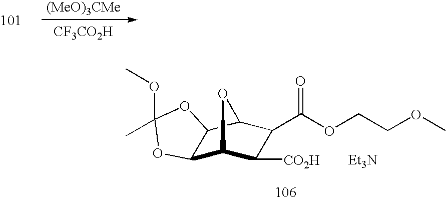

### Triethylammonium 2-methoxyethyl (1α,2α,3α,4α,5α,6α)-5,6-O-(1-methoxyethylydene)-5,6-dioxy-7-oxabicyclo[2.2.1]heptane-2,3-dicarboxylate (106)

A solution of compound 101 (2.76 g, 10.0 mmol), trimethyl orthoacetate (1.50 g, 12.5 mmol), and trifluoroacetic acid (0.023 g, 0.2 mmol) in DMF (50 mL) is kept overnight and neutralized with triethylamine (5 mL). The solvent is evaporated, and the residue is treated with ether (200 mL). The viscous precipitate is dried in vacuo and used in the next synthetic step without any further purification.

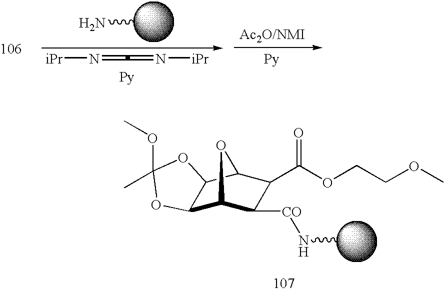

### Universal Solid Support 107

Aminoalkyl controlled pore glass (2667 mg, 0.5 mmol) is gently shaken with compound 106 (434 mg, 1.0 mmol), N,N′-diisopropylcarbodiimide (189 mg, 1.5 mmol), and 4-dimethylaminopyridine (61 mg, 0.5 mmol) for 24 h. The solid support is filtered off and washed with pyridine (3×20 mL). The solid support is additionally washed with ethyl acetate, dried, and capped by treating with a mixture of Ac₂O/pyridine/N-methylimidazole/THF (10:10:10:70) for 3 h at room temperature. Finally, the solid support 107 with a loading of 70 to 80 μmol g⁻¹ is washed with MeCN and ethyl acetate and dried.

### Example 42

### Universal Solid Support 108

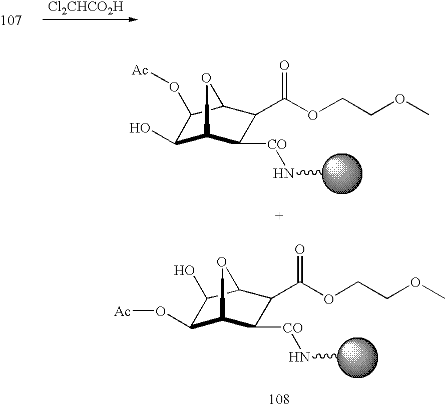

### Universal Solid Support 108

The solid support 107.0 g) is washed with 3% dichloroacetic acid (20 mL) for 5 min followed by washing with pyridine (3×10 mL). Finally, the solid support 123 is washed with MeCN and dried.

### Example 43

### Universal Solid Support 109

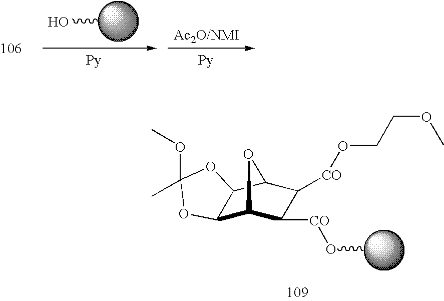

PS-PEG solid support (2500 mg, 0.5 mmol) is gently shaken with compound 106 (434 mg, 1.0 mmol), N,N′-diisopropylcarbodiimide (189 mg, 1.5 mmol), and 4-dimethylaminopyridine (61 mg, 0.5 mmol) for 24 h. The solid support is filtered off and washed with pyridine (3×20 mL). The solid support is additionally washed with ethyl acetate, dried, and capped by treating with a mixture of Ac₂O/pyridine/N-methylimidazole/THF (10:10:10:70) for 3 h at room temperature. Finally, the solid support 107 with a loading of 30 to 40 μmol g⁻¹ is washed with MeCN and ethyl acetate and dried.

### Example 44

### Universal Solid Support 115

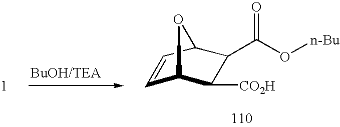

### Monobutyl (1α,2α,3α,4α,5α,6α)-7-oxabicyclo[2.2.1]hept-5ne-2,3-dicarboxylate (110)

A solution of commercial compound 1, (8.31 g, 50.0 mmol) and 4-dimethylaminopyridine (183 mg, 1.5 mmol) in n-butanol (5.56 g, 75.0 mmol), triethylamine (5.06 g, 50 mmol), and dioxane (50 mL) was kept overnight and evaporated. The residue was dissolved in water (50 mL) and acidified with conc. aqueous HCl. The product was extracted with ethyl acetate, dried over Na₂SO₄, and evaporated to give practically pure 110 (6.12 g, 100%).

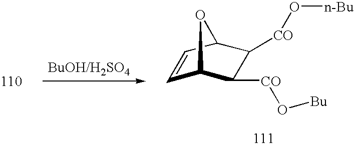

### Dibutyl (1α,2α,3α,4α,5α,6α)-5,6-dihydroxy-7-oxabicyclo[2.2.1]hept-5-ne-2,3-dicarboxylate (111)

A solution of compound 110 (2.53 g, 10.5 mmol) and sulfuric acid (0.2 g, 2 mmol) in CHCl₃ (100 mL) and n-butanol (8.1 g, 109 mmol) was refluxed for 2 h with a water separator filled with Na₂SO₄. The reaction mixture was washed with 5% aqueous NaHCO₃ (5×100 mL), dried over Na₂SO₄, and evaporated. The title compound was isolated by chromatography on a silica gel column.

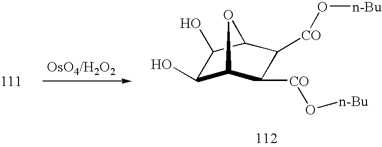

### Dibutyl (1α,2α,3α,4α,5α,6α)-5,6-dihydroxy-7-oxabicyclo[2.2.1]heptane-2,3-dicarboxylate (112)

A solution of compound 111 (14.80 g, 50.0 mmol) in hydrogen peroxide (30% aqueous, 5.7 g, 60.0 mmol), and MeCN (50 mL) was treated with osmium tetroxide (127 mg, 0.5 mmol) in t-butanol (10.0 mL) overnight at 28-30° C. The reaction mixture was evaporated, diluted with brine (90 mL) and extracted with ethyl acetate (5×100 mL). Extracts were dried over Na₂SO₄ and evaporated. The title compound was purified by column chromatography.

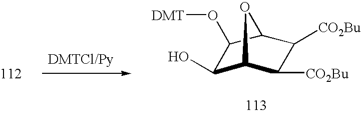

### Dibutyl (1α,2α,3α,4α,5α,6α)-5-hydroxy-6-(4,4′-dimethoxytrityloxy)-7-oxabicyclo[2.2.1]heptane-2,3-dicarboxylate (113)

A solution of compound 112 (3.30 g, 10 mmol) and 4,4′-dimethoxytrityl chloride (2.70 g, 8 mmol) in pyridine (50 mL) was kept overnight. The solvent was evaporated, the residue was treated with ethyl acetate (200 mL) and 5% aqueous NaHCO₃ (20 mL). The organic solution was washed with 5% aqueous NaHCO₃ (20 mL), dried over Na₂SO₄, and evaporated. The title compound was isolated by chromatography on a silica gel column.

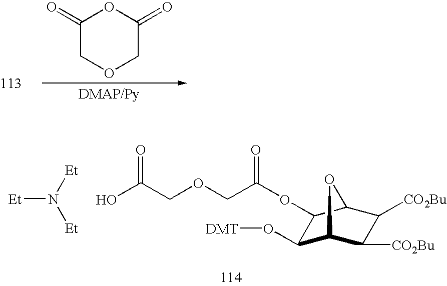

### Triethylammonium dibutyl 5-(4,4′-dimethoxytrityloxy)-6-[[(carboxymethyl)oxy]acetoxy]-7-oxabicyclo[2.2.1]heptane-2,3-dicarboxylate (114)

A solution of compound 113 (633 mg, 1.0 mmol), diglycolic anhydride (348 mg, 3.0 mmol), and 4-dimethylaminopyridine (122 mg, 1.0 mmol) in pyridine (50 mL) was kept overnight. The solvent was evaporated, the residue was treated with ethyl acetate (200 mL) and 1 M aqueous triethylammonium acetate (20 mL). The organic solution was washed with 1 M aqueous triethylammonium acetate (5×20 mL), water (3×20 mL) dried over Na₂SO₄, and evaporated. The residue was dissolved in ethyl acetate (20 mL) and precipitated into hexane (250 mL). The precipitate was collected, washed with hexane, and dried to give 114.

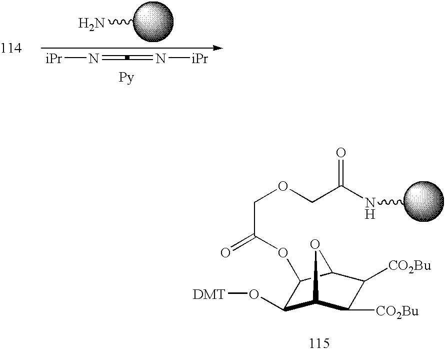

### Universal Solid Support (115)

A mixture of compound 114 (850 mg, 1.0 mmol), long chain aminoalkyl controlled pore glass (2667 mg, 0.5 mmol), and N,N′-diisopropylcarbodiimide (252 mg, 2.0 mmol) in pyridine (20 mL) was shaken overnight. The solid was filtered, washed with pyridine (5×10 mL) and capped with a mixture of Ac₂O/pyridine/N-methylimidazole/THF (10:10:10:70) for 3 h at room temperature. Finally, the solid support 115 wa washed with MeCN and ethyl acetate and dried to give a loading of 39.3 μmol g⁻¹.

### Example 45

### Universal Solid Support 116

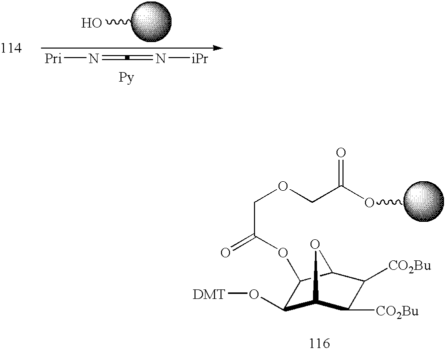

### Universal Solid Support 116

A mixture of compound 114 (850 mg, 1.0 mmol), PS-PEG solid support (2500 mg, 0.5 mmol), and N,N′-diisopropylcarbodiimide (252 mg, 2.0 mmol) in pyridine (20 mL) is shaken overnight. The solid is filtered, washed with pyridine (5×10 mL) and capped with a mixture of Ac₂O/pyridine/N-methylimidazole/THF (10:10:10:70) for 3 h at room temperature. Finally, the solid support 116 is washed with MeCN and ethyl acetate and dried to give a loading of 21.4 μmol g⁻¹.

### Example 46

### Universal Solid Support 119

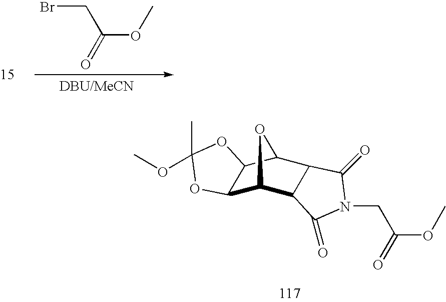

### (3aR,4R,5 S ,6R,7R,7aR)-rel-N-(Methoxycarbonylmethyl)-hexahydro-2-methyl-2-methoxy-4,7-epoxy-1,3-benzodioxole-5,6-dicarboximide (117)

A solution of compound 15 (510 mg, 2.00 mmol) and methyl bromoacetate (336 mg, 2.20 mmol) and DBU (167 mg, 2.2 mmol) in acetonitrile (5 mL) was stirred for 2 h. The mixture was evaporated and dissolved in ethyl acetate (50 mL). The solution was washed with water (3×25 mL) followed by brine (100 mL), dried over Na₂SO₄, and evaporated to give compound 117 (634 mg, 97.0%) in more than 97% purity. 327.29

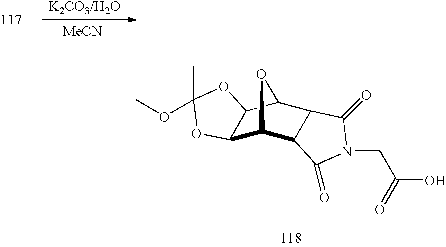

### (3aR,4R,5S,6R,7R,7aR)-rel-N-(carboxymethyl)-hexahydro-2-methyl-2-methoxy-4,7-epoxy-1,3-benzodioxole-5,6-dicarboximide (118)

A solution of compound 117 (655 mg, 2.00 mmol) in 50% aqueous MeCN containing 0.1 M K₂CO₃ (25 mL) was stirred for 24 h. The solvent was evaporated, the residue was treated with ethyl acetate (200 mL) and 1 M aqueous triethylammonium acetate (20 mL). The organic solution was washed with 1 M aqueous triethylammonium acetate (5×20 mL), water (3×20 mL) dried over Na₂SO₄, and evaporated. The residue was dissolved in ethyl acetate (20 mL) and precipitated into hexane (250 mL). The precipitate was collected, washed with hexane, and dried to give 118 (590 mg, 94.2%). 313.26

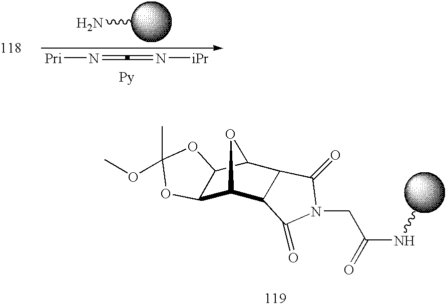

### Universal Solid Support 119

A mixture of compound 118 (475 mg, 1.23 mmol), long chain aminoalkyl controlled pore glass (5.00 g, 100 μmol g⁻¹, 0.5 mmol), N,N′-diisopropylcarbodiimide (233 mg, 1.85 mmol), and DMAP (12 mg, 0.1 mmol) in pyridine (25 mL) was shaken overnight. The solid phase was filtered, washed with pyridine (3×20 mL), acetonitrile (3×20 mL), and capped with a mixture of Ac₂O/pyridine/N-methylimidazole/THF (10:10:10:70; 50 mL) for 3 h. Finally, the solid phase was washed with MeCN and ethyl acetate and dried to give the solid support 119. The aliquot of the solid support 119 (10 mg) was detritylated and treated with a commercial 2-cyanoethyl T phosphoramidite in the presence of 1H-tetrazole using the standard protocol of oligonucleotide synthesis. The resulting solid support was dried and the loading of 39.7 μmol g⁻¹ was determined by the standard dimethoxytrityl assay.

### Example 47

### Universal Solid Support 22

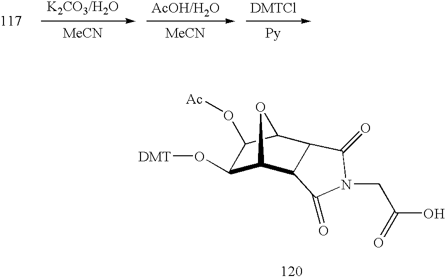

### Exo-N-carboxymethyl-5-acetoxy-6-(4,4′-dimethoxytrityloxy)-7-oxabicyclo[2.2.1]heptane-2,3-dicarboximide (120)

A solution of compound 117 (655 mg, 2.00 mmol) in 50% aqueous MeCN containing 0.1 M K₂CO₃ (25 mL) was stirred for 24 h. The solution was acidified with acetic acid, kept for 30 min, and evaporated to dryness. The residue was co-evaporated with pyridine (4×25 mL), dissolved in pyridine, and treated with 4,4′-dimethoxytrityl chloride (708 mg, 2.1 mmol) overnight. The solvent was evaporated, the residue was treated with ethyl acetate (200 mL) and 1 M aqueous triethylammonium acetate (20 mL). The organic solution was washed with 1 M aqueous triethylammonium acetate (5×20 mL), water (3×20 mL) dried over Na₂SO₄, and evaporated. The residue was purified on a silica gel column eluted with a step gradient of methanol in triethylamine and CH₂Cl₂ (5:95). The isolated product was dissolved in ethyl acetate (20 mL) and precipitated into hexane (250 mL). The precipitate was collected, washed with hexane, and dried to give 120 (886 mg, 73.7%). 601.23

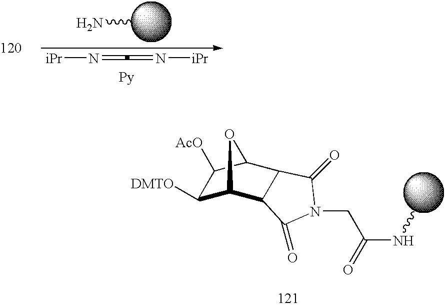

### Universal Solid Support 121

A mixture of compound 120 (721 mg, 1.2 mmol), long chain aminoalkyl controlled pore glass (6.00 g, 100 μmol g⁻¹, 0.60 mmol), N,N′-diisopropylcarbodiimide (252 mg, 2.0 mmol), and DMAP (12 mg, 0.1 mmol) in pyridine (30 mL) was shaken overnight. The solid phase was filtered, washed with pyridine (3×50 mL), acetonitrile (3×50 mL), and capped with a mixture of Ac₂O /pyridine/N-methylimidazole/THF (10:10:10:70; 50 mL) for 3 h. Finally, the solid phase was washed with MeCN and ethyl acetate and dried to give the solid support 121 loaded at 31.0 μmol g⁻¹.

### Example 48

### Universal Solid Support 122

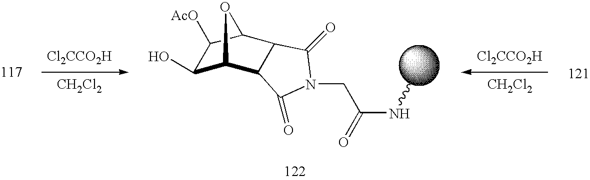

From universal solid support 119. The solid support 119 (500 mg) was washed on a filter with dichloroacetic acid (3% in CH2Cl2) for about 3 min until no orange-colored product was eluted from the solid support. The solid phase was filtered, washed with pyridine (3×20 mL), acetonitrile (3×20 mL) and dried to give the solid support 122.

From universal solid support 121. The solid support 121 (500 mg) was washed on a filter with dichloroacetic acid (3% in CH2Cl2) or a mixture of acetic acid, water, and MeCN (5:15:80) for about 3 min. The solid phase was filtered, washed with pyridine (3×20 mL), acetonitrile (3×20 mL) and dried to give the solid support 122.

### Example 49

### Universal Solid Support 121a

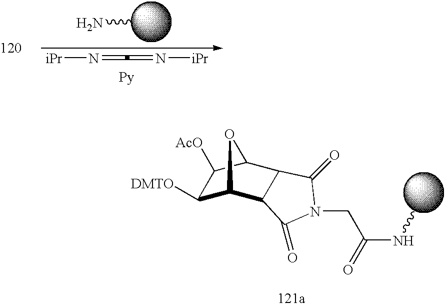

A mixture of compound 120 (721 mg, 1.2 mmol),aminomethyl polystyrene (5.00 g,), N,N′-diisopropylcarbodiimide (252 mg, 2.0 mmol), and DMAP (12 mg, 0.1 mmol) in pyridine (30 mL) was shaken overnight. The solid phase was filtered, washed with pyridine (3×50 mL), acetonitrile (3×50 mL), and capped with a mixture of Ac₂O/pyridine/N-methylimidazole/THF (10:10:10:70; 50 mL) for 3 h. Finally, the solid phase was washed with MeCN and ethyl acetate and dried to give the solid support 121 loaded at 25.7 μmol g⁻¹.

### Example 50

### Universal Solid Support 128

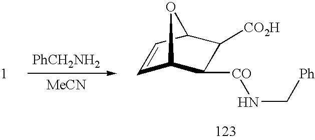

### Exo-N-benzyl-7-oxabicyclo[2.2.1]hept-5-ene-3-carboxamide-2-carboxylic acid (123)

A solution of compound 1 (8.31 g, 50.0 mmol) in MeCN (100 mL) was treated with benzylamine (5.46 g, 51.0 mmol) in an ice-water bath for 45 min and at room temperature for 2 h. The precipitate was filtered off and washed on the filter with MeCN (3×50 mL). The precipitate was dried in vacuo to give ca. 98% pure compound 123 (13.31 g, 97.1%), which was used in the next synthetic step without any further purification.

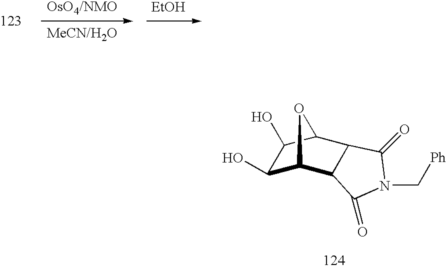

### Exo-N-benzyl-5,6-dihydroxy-7-oxabicyclo[2.2.1]heptane-2,3-dicarboximide (124)

A solution of compound 123 (6.83 g, 25.0 mmol) and N-methylmorpholine-N-oxide (3.08 g, 26.3 mmol) in acetonitrile (20.0 mL) and water (7.5 mL) was treated with osmium tetroxide (6 mg, 0.025 mmol) in t-butanol (0.33 mL) for 1 h at 60-65° C. The reaction mixture was evaporated and co-evaporated with acetonitrile (2×50 mL). The residue was refluxed in ethanol (45 mL) for 3 h. The mixture was diluted with ethanol (40 mL) and water (15 mL) and filtered through a short pad of silica gel. Silica gel was washed with 85% aqueous ethanol (75 mL), and the combined solution was evaporated. The solid residue was re-crystallized to give compound 124 (6.38 g, 88.2%) as an off-white solid.

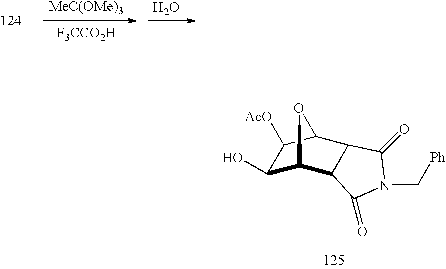

### Exo-N-benzyl-5-hydroxy-6-acetoxy-7-oxabicyclo[2.2.1]heptane-2,3-dicarboximide (125)

A mixture of compound 124 (2.89 g, 10.0 mmol), trimethyl orthoacetate (1.38 g, 11.5 mmol), trifluoroacetic acid (30 mg), and acetonitrile (50 mL) was stirred overnight and treated with water (2 mL). The solvent was evaporated, and the solid residue was recrystallized to give compound 125 (2.83 g, 85.4%).

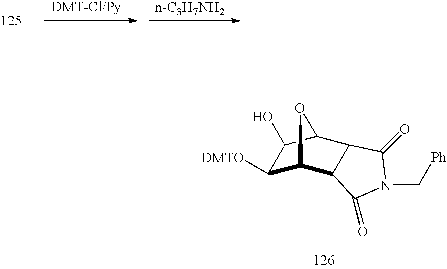

### Exo-N-benzyl-5-hydroxy-6-(4,4′-dimethoxytrityloxy)-7-oxabicyclo[2.2.1]heptane-2,3-dicarboximide (126)

A mixture of compound 125 (4.97 g, 15.0 mmol), dimethoxytrityl chloride (5.33 g, 15.8 mmol), and pyridine (70 mL) was shaken overnight and treated with n-propylamine (2 mL) for 3 h. The solvent was evaporated, the residue was dissolved in ethyl acetate (200 mL) and washed with 5% aqueous NaHCO₃ (3×20 mL) followed by brine (20 mL). The organic phase was dried over Na₂SO₄ and evaporated. The residue was purified on a silica gel column eluting with a gradient of MeOH in CH₂Cl₂ to give compound 126 (8.10 g, 91.3%).

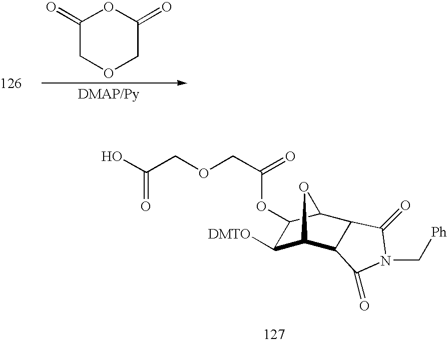

### Exo-N-benzyl-5-(diglycolyloxy)-6-(4,4′-dimethoxytrityloxy)-7-oxabicyclo[2.2.1]heptane-2,3-dicarboximide (127)

A solution of compound 126 (6.51 g, 11.0 mmol), diglycolic anhydride (2.55 g, 22.0 mmol), and 4-dimethylaminopyridine (61 mg, 0.5 mmol) in pyridine (100 mL) was kept overnight. The solvent was evaporated, the residue was treated with ethyl acetate (200 mL) and 1 M aqueous triethylammonium acetate (20 mL). The organic solution was washed with 1 M aqueous triethylammonium acetate (4×20 mL), water (3×20 mL) dried over Na₂SO₄, and evaporated. The residue was dissolved in ethyl acetate (50 mL) and precipitated into hexane (500 mL). The precipitate was collected, washed with hexane, and dried in vacuo to give 127 (7.28 g, 93.5%) as an off-white solid.

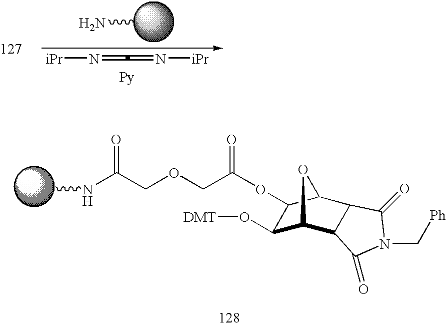

### Universal Solid Support 128

A mixture of compound 127 (708 mg, 1.0 mmol), long chain aminoalkyl controlled pore glass (4.27 g, 117 μmol g⁻¹, 0.5 mmol), N,N′-diisopropylcarbodiimide (189 mg, 1.5 mmol), and DMAP (12 mg, 0.1 mmol) in pyridine (20 mL) was shaken overnight. The solid phase was filtered, washed with pyridine (5×20 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 128 loaded at 42.2 μmol g⁻¹.

### Example 51

### Universal Solid Support 128a

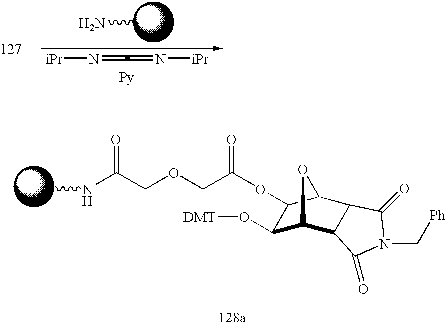

A mixture of compound 127 (708 mg, 1.0 mmol), aminomethyl polystyrene (5.0 g), N,N′-diisopropylcarbodiimide (189 mg, 1.5 mmol), and DMAP (12 mg, 0.1 mmol) in pyridine (20 mL) was shaken overnight. The solid phase was filtered, washed with pyridine (5×20 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 128a loaded at 29.6 μmol g⁻¹.

### Example 52

### Universal Solid Support 130

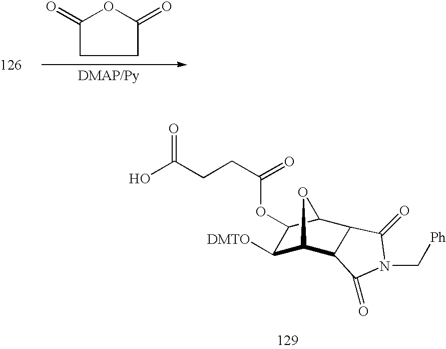

### Exo-N-benzyl-5-(succinyloxy)-6-(4,4′-dimethoxytrityloxy)-7-oxabicyclo[2.2.1]heptane-2,3-dicarboximide (129)

A solution of compound 126 (6.51 g, 11.0 mmol), succinic anhydride (2.20 g, 22.0 mmol), and 4-dimethylaminopyridine (61 mg, 0.5 mmol) in pyridine (100 mL) was kept overnight. The solvent was evaporated, the residue was treated with ethyl acetate (200 mL) and 1 M aqueous triethylammonium acetate (20 mL). The organic solution was washed with 1 M aqueous triethylammonium acetate (4×20 mL), water (3×20 mL) dried over Na₂SO₄, and evaporated. The residue was dissolved in ethyl acetate (50 mL) and precipitated into hexane (500 mL). The precipitate was collected, washed with hexane, and dried in vacuo to give 129 (6.85 g, 90.0%) as an off-white solid.

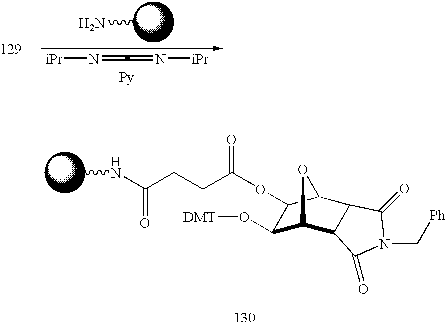

### Universal Solid Support 130

A mixture of compound 129 (692 mg, 1.0 mmol), long chain aminoalkyl controlled pore glass (4.27 g, 117 μmol g⁻¹, 0.5 mmol), N,N′-diisopropylcarbodiimide (189 mg, 1.5 mmol), and DMAP (12 mg, 0.1 mmol) in pyridine (20 mL) was shaken overnight. The solid phase was filtered, washed with pyridine (5×20 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 130 loaded at 46.2 μmol g⁻¹.

### Example 53

### Universal Solid Support 130a

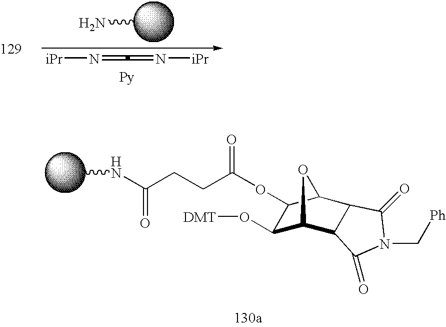

A mixture of compound 129 (708 mg, 1.0 mmol), aminomethyl polystyrene (5.0 g), N,N′-diisopropylcarbodiimide (189 mg, 1.5 mmol), and DMAP (12 mg, 0.1 mmol) in pyridine (20 mL) was shaken overnight. The solid phase was filtered, washed with pyridine (5×20 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 130a loaded at 31.9 μmol g⁻¹.

### Example 54

### Universal Solid Support 130b

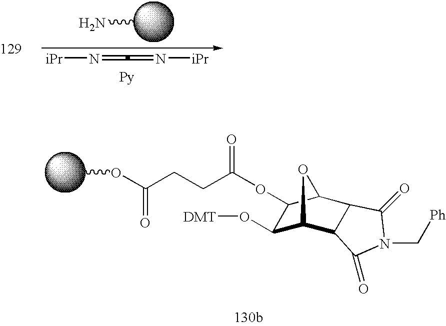

A mixture of compound 129 (708 mg, 1.0 mmol), polyethyleneglycol-grafted polystyrene (5.0 g), N,N′-diisopropylcarbodiimide (189 mg, 1.5 mmol), and DMAP (12 mg, 0.1 mmol) in pyridine (20 mL) was shaken overnight. The solid phase was filtered, washed with pyridine (5×20 mL), and capped with a mixture of Ac₂O/pyridine/N-methylimidazole/THF (10:10:10:70; 40 mL) for 3 h. Finally, the solid phase was washed with MeCN and ethyl acetate and dried to give the solid support 130b loaded at 19.1 μmol g⁻¹.

### Example 55

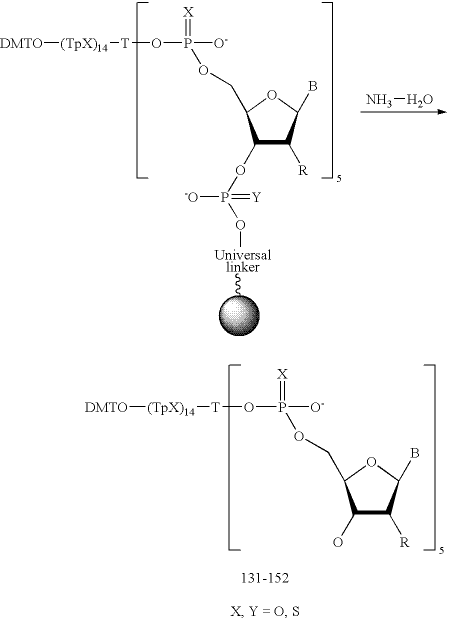

### Synthesis of oligonucleotides 117-138 on Universal Solid Supports 5, 6, 9, 12, 13, 19, 22, 23, 29-33, 34a, 40, 46, 47, 53, 54, 57-60, 65, 66, 68a, 70a, 73-76, 78, 83, 84, 89, 90, 96, 97, 99 105, 107-109, 115, 116, 119, 121, 121a, 122, 128, 128a, 130, 130a, and 130b

The oligonucleotide synthesis is performed on an ABI 380B DNA Synthesizer on a 1 to 4 μmol scale according to the manufacturer's recommendations. The standard 2′-deoxy, 2′-O-methyl, 2′-fluoro, and 2′-O-(t-butyldimethylsilyl) phosphoramidites are used as 0.1 M solutions in anhydrous MeCN. The oxidation step is carried out with the standard iodine reagent or with t-butyl hydroperoxide (10% in MeCN) for 10 min. The preparation of oligonucleotide phosphorothioates is carried out using 3H-1,2-benzodithiol-3-one 1,1-dioxide (0.05 M in MeCN) as a sulfur-transfer reagent. Optionally, oligonucleotide phosphorothioates are synthesized using oxidation with the standard iodine reagent or t-butyl hydroperoxide solution for the linkage between the solid support and the 3′-terminal nucleoside while the internucleosidic linkages are sulfurized in a conventional manner.

The coupling time of 10 min is used for the attachment of the 3′-terminal nucleoside residues to universal solid supports. The structures of synthesized oligonucleotides are given above, the detailed description of groups B, R, X, and Y is disclosed in Table 1.

### Example 56

The release of oligonucleotides synthesized on universal solid supports 19, 22, 23, 29, 30-33, 40, 46, 47, 59, 60, 65, 66, 73, 74, 83, 84, 89, 90, 96, 97, 99, 109, 115, 116, 128, 128a, 130, 130a, and 130b.

The solid support-bound oligonucleotides are quantitatively released with concentrated aqueous ammonium hydroxide in 60 min at room temperature. The base deprotection is then completed as recommended for the protection groups used in phosphoramidites.

### Example 57

The release of oligonucleotides synthesized on universal solid supports 5, 6, 9, 12, 13, 34a, 53, 54, 57, 58, 68a, 70a, 75, 76, 78, 105, 107, 108, 119, 121, 121a, 122.

The solid support-bound oligonucleotides are quantitatively released with concentrated aqueous ammonium hydroxide in 6 h at room temperature. The base deprotection is then completed as recommended for the protection groups used in phosphoramidites. Alternatively, the solid support-bound oligonucleotides are treated with concentrated ammonium hydroxide for 6 h at 55° C., which completes the release and the deprotection of nucleic bases.

### Example 58

Synthesis and the release of oligonucleotides synthesized using universal phosphoramidites 34,68,70.

The universal phosphoramidites phosphoramidites 34, 68, 70 are coupled to a hydroxyalkyl solid support prepared as reported previously (Hovinen, J.; Guzaev, A; Azhayev, A.; Lonnberg, H. Tetrahedron Lett. 1993, 34, 8169-8172) or to PEG-PS solid support prior to the attachment of nucleosidic phosphoramidites. Universal phosphoramidites are used as 0.1 M solutions in anhydrous MeCN. The oxidation step is carried out with the standard iodine reagent or with t-butyl hydroperoxide (10% in MeCN) for 10 min.

The solid support-bound oligonucleotides are quantitatively released with concentrated aqueous ammonium hydroxide in 6 h at room temperature. The base deprotection is then completed as recommended for the protection groups used in phosphoramidites. Alternatively, the solid support-bound oligonucleotides are treated with concentrated ammonium hydroxide for 6 h at 55° C., which completes the release and the deprotection of nucleic bases.

TABLE 1
Oligonucleotides 131-152 synthesized on universal solid
supports or with the use of universal phosphoramidites.

| Oligonucleotide | | | | |
|---|---|---|---|---|
| Compound | B | R | X | Y |
| 131 | A | H | O | O |
| 132 | G | H | O | O |
| 133 | C | H | O | O |
| 134 | T | H | O | O |
| 135 | A | H | S | S |
| 136 | G | H | S | S |
| 137 | G | H | S | O |
| 138 | C | H | S | S |
| 139 | T | H | S | S |
| 140 | G | OMe | O | O |
| 141 | U | OMe | O | O |
| 142 | A | OMe | S | S |
| 143 | G | OMe | S | S |
| 144 | G | OMe | S | O |
| 145 | C | OMe | S | S |
| 146 | U | OMe | S | S |
| 147 | C | F | O | O |
| 148 | U | F | O | O |
| 149 | A | OH | O | O |
| 150 | G | OH | O | O |
| 151 | C | OH | O | O |
| 152 | U | OH | O | O |

What is claimed is:

1. A compound of formula I: 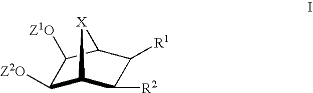
wherein:
X is O or NR³;
R³ is -L-sm, alkyl, —C(═O)alkyl, —C(═O)aryl, —C(═O)NH-alkyl, —C(═O)NH-aryl or an amino protecting group;
L is a linking moiety;
sm is a support medium;
R¹ and R² are independently H, alkyl, —C(═O)—R⁴; or R¹ and R² are fused to form a ring structure so that R¹+R² is —C(═O)—N(R⁵)—C(═O)—; or R¹ and R² together with the carbon atoms they are attached to form a substituted or unsubstitute cycloalkyl, a substituted or unsubstituted aryl, a substituted or unsubstituted heterocycloalkyl, or a substituted or unsubstituted heteroaryl; or one of R₁ and R₂ is -L-sm and the other of R¹ and R² is H, O—C(═O)R⁶, or —C(═O)—R⁴;
R⁴ is —O(alkyl), —O(benzyl), —O(alkoxyalkyl), or —N(J¹)J²;
J¹ is H or alkyl;
J² is H, alkyl, benzyl, alkoxyalkyl, —(CH₂)ₙ—O-L-sm, or a nitrogen-protecting group;
n is an integer from 0 to about 12;
or J¹ and J² together with the nitrogen atom they are attached to form a heteroaryl or heterocycloalkyl;
R⁵ is alkyl, aryl, benzyl, alkoxyalkyl, —(CH₂)ₙ-L-sm, or nitrogen-protecting group;
R⁶ is CH₂-G¹;
Z¹ and Z² are independently H, or orthogonal hydroxy protecting groups; or one of Z¹ or Z² is H and the other of Z¹ or Z² is —C(═O)CH₂G¹; or one of Z¹ or Z² is H or hydroxy protecting group and the other of Z¹ or Z² is -L-sm;
or Z₁ and Z₂ together with the oxygen atoms they are attached to and the carbon atoms the oxygen atoms are attached to form a ring structure wherein Z¹+Z² is —C(OAlkyl)(CH₂G¹)-;
G¹, for each occurrence, is independently, H, alkyl, aryl, acetyl, acetonyl, or an electron-withdrawing group;
provided that when one of R¹ or R² is -L-sm and the other of R¹ and R² is O—C(═O)R⁶ or —C(═O)—R⁴, then Z¹ and Z² together with the oxygen atoms they are attached to and the carbon atoms the oxygen atoms are attached to form a ring structure wherein Z¹+Z² is —C(OAlkyl)(CH₂G¹)-; and provided that the compound includes one -L-sm.

2. The compound of claim 1, wherein L is —C(═O)—, —CH₂OC(═O)—, —O—C(═O)—, —P(OR⁷)(═O)—, —P(OR⁷)(═S)—, —P(O(CH₂)₂CN)(═O)—, or —P(O(CH₂)₂CN)(═S)—;
R⁷ is a negative charge, alkyl, cycloalkyl, or phosphate protecting group;

3. The compound of claim 1, having Formula II: 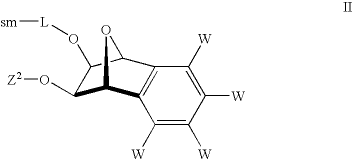
wherein:
Z² is 4,4′-dimethoxytrityl group, 4,4′,4″-trimethoxytrityl group, or H; and
W, for each occurrence, is independently H or a halogen atom.

4. The compound of claim 3, wherein each W is a halogen atom.

5. The compound of claim 1 having Formula III: 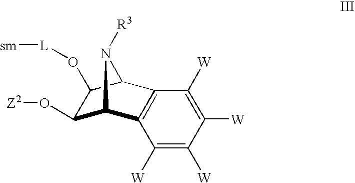
wherein:
R³ is alkyl, —C(═O)alkyl, —C(═O)NH(Alkyl), —C(═O)NH(Aryl) or a nitrogen protecting group;
Z² is 4,4′-dimethoxytrityl group, 4,4′,4″-trimethoxytrityl group, or H; and
W, for each occurrence, is independently H or a halogen atom.

6. The compound of claim 5, wherein each W is a halogen atom.

7. The compound of claim 1 having one of Formulas IVa or IVb: 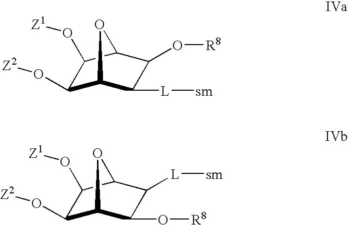
wherein:
R⁸ is —C(═O)CH₂-G¹
one of Z¹ and Z² is —C(═O)CH₂-G¹ and the other of Z¹ and Z² is 4,4′-dimethoxytrityl group, 4,4′,4″-trimethoxytrityl group, or H; or Z¹ and Z² together with the oxygen atoms they are attached to and the carbon atoms the oxygen atoms are attached to form a ring structure so that Z¹+Z² is —C(OAlkyl)(CH₂G¹)-.

8. The compound of claim 7, wherein G¹ is H, Cl, acetyl, acetonyl, OCH₃, or —OC₆H₅.

9. The compound of claim 1 having one of Formulas Va or Vb: 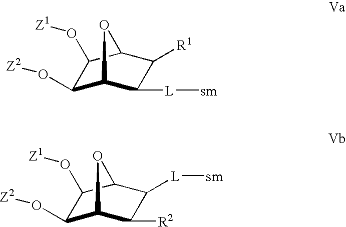
wherein:
R¹ and R² are each, independently, H or —C(═O)—R⁴;
one of Z¹ or Z² is H and the other of Z¹ or Z² is —C(═O)CH₂G¹; or Z¹ and Z² together with the oxygen atoms they are attached to and the carbon atoms the oxygen atoms are attached to form a ring structure so that Z¹+Z² is —C(OAlkyl)(CH₂G¹)-.

10. The compound of claim 9, wherein L is —C(═O)—, —CH₂OC(═O)—, —O—C(═O)—, —P(OR⁷)(═O)—, —P(OR⁷)(═S)—, —P(O(CH₂)₂CN)(═O)—, or —P(O(CH₂)₂CN)(═S)—;
R⁷ is a negative charge, alkyl, cycloalkyl or phosphate protecting group.

11. The compound of claim 9, wherein G¹ is H, acetyl, acetonyl, Cl, OCH₃, or —OC₆H₅.

12. The compound of claim 1 having one of Formulas VIa or VIb: 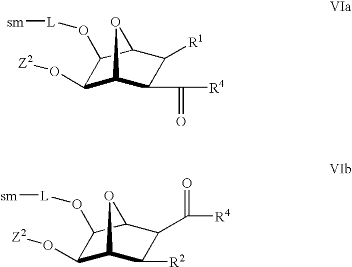
wherein:
R¹ and R² are each, independently, H or —C(═O)—R⁴; and
Z² is 4,4′-dimethoxytrityl group, 4,4′,4″-trimethoxytrityl group, or H.

13. The compound of claim 12, wherein L is —C(═O)—.

14. The compound of claim 1 having Formula VII: 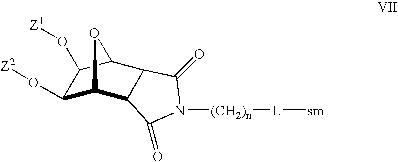
wherein:
L is —OC(═O)—, —C(═O)— or —OP(OR⁷)(═Y)—;
R⁷ is a negative charge, alkyl, cycloalkyl, or phosphate protecting group;
Y is O or S; and
one of Z¹ and Z² is —C(═O)CH₂-G¹ where G¹ is H, an alkyl group, or an electron-withdrawing group and the other of Z¹ and Z² is 4,4′-dimethoxytrityl group, 4,4′,4″-trimethoxytrityl group, or H; or Z¹ and Z² together with the oxygen atoms they are attached to and the carbon atoms the oxygen atoms are attached to form a ring structure so that Z¹+Z² is —C(OAlkyl)(CH₂G¹)-.

15. The compound of claim 14, wherein G¹ is H, Cl, acetyl, acetonyl, OCH₃, or —OC₆H₅.

16. The compound of claim 1 having Formula VIII: 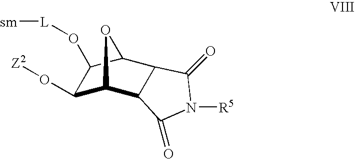
wherein:
Z² is 4,4′-dimethoxytrityl group, 4,4′,4″-trimethoxytrityl group, or H.

17. The compound of claim 16, wherein R⁵ is methyl, ethyl, propyl, iso-propyl, phenyl, or benzyl group.

18. The compound of claim 16, wherein L is —C(═O)—.

19. The compound of claim 1, wherein said support medium is glass surfaces or particles, polymers, or soluble support media.

20. The compound of claim 19, wherein said support medium is glass surfaces or particles, controlled pore glass, succinyl and diglycolyl controlled pore glass, controlled pore glass derivatized with 1,2-phenylenedioxydiacetic acid and/or 1,4-phenylenedioxydiacetic acid, polystyrene, copolymers of styrene, copolymers of styrene and divinylbenzene, or polystyrene grafted with polyethyleneglycol.

21. The compound of claim 1 wherein one of Z₁ and Z₂ is triethylsilyl, triisopropylsilyl, t-butyldimethylsilyl, t-butyldiphenylsilyl, triphenylsilyl, formyl, benzoylformyl, acetyl, methoxyacetyl, phenoxyacetyl, chloroacetyl, dichloroacetyl, trichloroacetyl, trifluoroacetyl, benzoyl, p-phenylbenzoyl, 9-fluorenylmethoxycarbonyl, levulinyl, acetoacetyl groups, or -L-sm, and the other of Z₁ and Z₂ is H, 4,4′,4″-trimethoxytrityl (TMT), 4,4′-dimethoxytrityl (DMT), 4-methoxytrityl, triphenylmethyl (trityl), 9-phenylxanthen-9-yl (Pixyl), 9-(4-methoxyphenyl)xanthen-9-yl (Mox), 2,7-dimethyl-9-phenylxanthen-9-yl, 2,7-dimethyl-9-(4-methoxyphenyl)xanthen-9-yl, tetrahydropyranyl, 1-ethoxyethyl, 2-trimethylsilylethyl, triethylsilyl, triisopropylsilyl, t-butyldimethylsilyl, t-butydiphenylsilyl, triphenylsilyl, bis(trimethylsilyloxy)cyclooctyloxysilyl, bis(trimethylsilyloxy)cyclododecyloxysilyl, p-phenylazophenyloxycarbonyl (PAPoc), 9-fluorenylmethoxycarbonyl (Fmoc), 2,4-dinitrophenylethoxycarbonyl (DNPEoc), (dialkoxy)alkylmethyl including but not limited to bis(2-acetoxyethoxy)methyl (ACE), levulinyl, or acetoacetyl groups.

22. A method for functionalizing a support medium with a first monomeric subunit, comprising the steps of:
a) providing a support-bound compound of claim 1;
b) optionally, deblocking one of said orthogonal protecting groups Z¹ and Z² to give a hydroxy group or converting said hydroxy protecting group Z¹+Z² to Z¹ and Z² wherein one of Z¹ and Z² is H and the other of Z¹ and Z² is —C(═O)(CH₂G¹); and
c) treating said hydroxy group with a first monomeric subunit having an activated phosphorus group and a further protected hydroxy group thereon for a time and under conditions sufficient to form a monomer-functionalized support medium.

23. The method of claim 22 further comprising:
d) treating said monomer-functionalized support medium with a capping agent; and
e) optionally, treating said monomer-functionalized support medium with an oxidizing or sulfurizing agent.

24. The method of claim 23 further comprising:
f) deblocking said further protected hydroxy group to give a hydroxy group;
g) treating the hydroxy group with a further monomeric subunit having an activated phosphorus group and a further protected hydroxy group thereon for a time and under conditions sufficient to form an extended compound;
h) treating said extended compound with a capping agent;
i) optionally, treating said extended support-bound compound with an oxidizing or sulfurizing agent;
j) repeating steps f) through i) one or more times to form a further extended compound.

25. The method of claim 24 further comprising steps of:
k) optionally, selectively deblocking the other of said orthogonal hydroxy protecting groups Z¹ and Z² with a specific deblocking agent to give a hydroxy group; and
l) releasing said oligomeric compound from solid support to solution with a basic reagent effective to cleave said oligomeric compound from said support medium.

26. The method of claim 25, wherein said selective deblocking step affects no cleavage of phosphate or thiophosphate protecting groups.

27. The method of claim 25, wherein said specific deblocking agent is a solution of hydrazinium or N-methylhydrazinium salt in aqueous or organic media.

28. The method of claim 25, wherein said releasing step is effective to remove protecting groups present on said oligomeric compound.

29. The method of claim 25, wherein said cleaved oligomeric compound has a terminal hydroxy group at the site of cleavage.

30. The method of claim 22 wherein said support medium is glass surfaces or particles, polymers, or soluble support media.

31. The method of claim 22 wherein said support medium is controlled pore glass, succinyl and diglycolyl controlled pore glass, polystyrene, copolymers of styrene, copolymers of styrene and divinylbenzene, polystyrene grafted with polyethyleneglycol.

32. The method of claim 22, wherein the treating step of said reactive hydroxy group with a monomeric subunit having an activated phosphorus group and a further protected hydroxy is performed in the presence of an activating agent.

33. The method of claim 22, wherein said monomeric subunit having an activated phosphorus group is a phosphoramidite, an H-phosphonate or a phosphate triester.

34. The method of claim 22, wherein one of said groups Z₁ and Z₂ is an acid labile hydroxy protecting group.

35. The method of claim 22, wherein one of said groups Z₁ and Z₂ is hydrogen.

36. The method of claim 22, wherein each of said further hydroxy protecting groups are acid labile.

37. The method of claim 34, wherein said one of said groups Z₁ and Z₂ and each of said further hydroxy protecting groups are removed by contacting said hydroxy protecting groups with an acid, wherein the acid is formic acid, acetic acid, chloroacetic acid, dichloroacetic acid, trichloroacetic acid, trifluoroacetic acid, benzenesulfonic acid, toluenesulfonic acid, or phenylphosphoric acid.

38. The method of claim 24, wherein the oligomeric compounds may be oligonucleotides, modified oligonucleotides, oligonucleotide analogs, oligonucleosides, oligonucleotide mimetics, short interfering RNA, aptamers, hemimers, gapmers and chimeras.

39. The method of claim 38, wherein said oligomeric compounds include nucleotide chain having from 1 to about 200 monomeric subunits.

40. A compound of formula Ia: 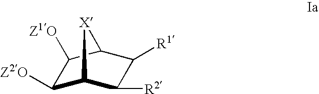
wherein:
X′ is O or NR^{3′};
R^{3′} is -L-R⁹, alkyl, —C(═O)alkyl, —C(═O)aryl, —C(═O)NH-alkyl, —C(═O)NH-aryl or an amino protecting group;
L is a linking moiety;
R⁹ is —X²—P(X₃R⁷)NJ³J⁴;
X² and X³ are each, independently, O or S;
R⁷ is a negative charge, alkyl, cycloalkyl or phosphate protecting group;
J³ and J⁴ are each, independently, and alkyl, a cycloalkyl, or an arylalkyl, or J³ and J⁴ together with the nitrogen atom they are attached to form a heteroaryl or heterocycloalkyl;
R^{1′} and R^{2′} are independently H, alkyl, —C(═O)—R⁴; or R^{1′} and R^{2′} are fused to form a ring structure so that R^{1′}+R^{2′} is —C(═O)—N(R⁵)—C(═O)—; or R^{1′} and R^{2′} together with the carbon atoms they are attached to form a substituted or unsubstitute cycloalkyl, a substituted or unsubstituted aryl, a substituted or unsubstituted heterocycloalkyl, or a substituted or unsubstituted heteroaryl; or one of R^{1′} and R^{2′} is -L-R⁹ and the other of R¹ and R² is H, O—C(═O)R⁶, or —C(═O)—R⁴;
R⁴ is —O(alkyl), —O(benzyl), —O(alkoxyalkyl), or —N(J¹)J²;
J¹ is H or alkyl;
J² is H, alkyl, benzyl, alkoxyalkyl, —(CH₂)ₙ—O-L-sm, or a nitrogen-protecting group;
n is an integer from 0 to about 12;
or J¹ and J² together with the nitrogen atom they are attached to form a heteroaryl or heterocycloalkyl;
R⁵ is alkyl, aryl, benzyl, alkoxyalkyl, —(CH₂)ₙ-L-sm, or nitrogen-protecting group;
R⁶ is CH₂-G¹;
Z^{1′} and Z^{2′} are independently H, or orthogonal hydroxy protecting groups; or one of Z^{1′} and Z^{2′} is H or hydroxy protecting group and the other of Z^{1′} and Z^{2′} is -L-R⁹;
or Z¹′ and Z²′ together with the oxygen atoms they are attached to and the carbon atoms the oxygen atoms are attached to form a ring structure wherein Z^{1′}+Z^{2′} is —C(OAlkyl)(CH₂G¹)-;
G¹, for each occurrence is, independently, H, alkyl, aryl, acetyl, acetonyl, or an electron-withdrawing group;
provided that the compound includes one -L-R⁹.
